(19) United States
(12) Patent Application Publication
(10) Pub. No.: US 2024/0277806 A1
(43) Pub. Date: Aug. 22, 2024

(54) ACTRII PROTEINS AND USES THEREOF

(71) Applicant: Acceleron Pharma Inc., Cambridge, MA (US)

(72) Inventors: Gang Li, Cambridge, MA (US); Patrick Andre, Cambridge, MA (US); Ravindra Kumar, Cambridge, MA (US)

(21) Appl. No.: 18/568,112

(22) PCT Filed: Jun. 10, 2022
(86) PCT No.: PCT/US2022/033007

§ 371 Date: Dec. 7, 2023

Related U.S. Application Data
(60) Provisional application No. 63/209,871, filed on Jun. 11, 2021.

(51) Int. Cl. A61K 38/17; A61P 9/12

(52) U.S. Cl. CPC A61K 38/179; A61P 9/12

(57) ABSTRACT

In some aspects, the disclosure relates to compositions and methods comprising ActRII polypeptides to treat, prevent, or reduce the progression rate and/or severity of pulmonary hypertension associated with lung disease (e.g, pulmonary hypertension associated with chronic obstructive pulmonary disease (COPD), interstitial lung disease (ILD), or combined pulmonary fibrosis and emphysema (CPFE)), particularly treating, preventing or reducing the progression rate and/or severity of one or more pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) associated complications.

### CROSS-REFERENCE TO RELATED APPLICATIONS

This application claims the benefit of and priority to U.S. Provisional Application No. 63/209,871, filed Jun. 11, 2021. The foregoing application is incorporated herein by reference in its entirety.

### FIELD OF INVENTION

This application relates to ActRII polypeptides, compositions and methods comprising ActRII polypeptides to treat, prevent, or reduce the progression rate and/or severity of pulmonary hypertension associated with lung disease (e.g., pulmonary hypertension associated with chronic obstructive pulmonary disease (COPD), interstitial lung disease (ILD), or combined pulmonary fibrosis and emphysema (CPFE)), particularly treating, preventing or reducing the progression rate and/or severity of one or more pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) associated complications.

### BACKGROUND OF THE INVENTION

Pulmonary hypertension (PH) is a disease characterized by high blood pressure in lung vasculature, including pulmonary arteries, pulmonary veins, and pulmonary capillaries. In general, PH is defined as a mean pulmonary artery pressure (mPAP) ≥20 mm Hg at rest or ≥30 mm Hg with exercise [Hill et al., Respiratory Care 54(7):958-68 (2009)]. One of the main PH symptoms is difficulty in breathing or shortness of breath, and other symptoms include fatigue, dizziness, fainting, peripheral edema (swelling in foot, legs or ankles), bluish lips and skin, chest pain, angina pectoris, light-headedness during exercise, non-productive cough, racing pulse and palpitations. PH can be a severe disease causing heart failure, which is one of the most common causes of death in people who have pulmonary hypertension. Postoperative pulmonary hypertension may complicate many types of surgeries or procedures, and present a challenge associated with a high mortality.

PH may be grouped based on different manifestations of the disease sharing similarities in pathophysiologic mechanisms, clinical presentation, and therapeutic approaches [Simonneau et al., JACC 54(1):S44-54 (2009)]. Clinical classification of PH was first proposed in 1973, and a recent updated clinical classification was endorsed by the World Health Organization (WHO) in 2018. According to the updated PH clinical classification, there are five main groups of PH: pulmonary arterial hypertension (PAH), characterized by a pulmonary artery wedge pressure (PAWP) ≤15 mm Hg; PH due to left heart disease (also known as pulmonary venous hypertension or congestive heart failure), characterized by a PAWP >15 mm Hg; PH due to lung diseases and/or hypoxia; PH due to pulmonary artery obstructions; and PH with unclear and/or multifactorial etiologies [Simonneau et al., JACC 54(1):S44-54 (2009); Hill et al., Respiratory Care 54(7):958-68 (2009)]. PAH is further classified into idiopathic PAH (IPAH), a sporadic disease in which there is neither a family history of PAH nor an identified risk factor, heritable PAH; PAH induced by drugs and toxins; PAH associated with connective tissue diseases, HIV infection, portal hypertension, congenital heart diseases, schistosomiasis, and chronic hemolytic anemia; and persistent PH of newborns [Simonneau et al., (2019) Eur Respir J: 53:1801913]. Diagnosis of various types of PH requires a series of tests.

In general, PH treatment depends on the cause or classification of PH. Where PH is caused by a known medicine or medical condition, it is known as a secondary PH, and its treatment is usually directed at the underlying disease. Treatment of Group 3 pulmonary hypertension has traditionally been to optimize treatment of the underlying lung disease and give long-term oxygen therapy to those who are hypoxic. The efficacy of pulmonary vasodilators in this group of patients is unclear. Furthermore, there have been mixed results from meta-analysis assessing the effects of vasodilators on exercise tolerance and quality of life. More studies are required in order to establish the groups of patients who stand to most benefit from vasodilator therapy but the current advice is treat the lung, not the pressure. See. e.g., McGettrick M. et al., Glob Cardiol Sci Pract. 2020 Apr. 30; 2020(1).

There is a high, unmet need for effective therapies for treating pulmonary hypertension. Accordingly, it is an object of the present disclosure to provide methods for treating, preventing, or reducing the progression rate and/or severity of PH, particularly treating, preventing or reducing the progression rate and/or severity of one or more PH-associated complications.

### SUMMARY OF THE INVENTION

In certain aspects, the disclosure provides a method of treating pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the method reduces the right ventricular systolic pressure (RVSP) by at least 10%.

In certain aspects, the disclosure provides a method of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the one or more complications of pulmonary hypertension associated with lung disease is selected from the group consisting of persistent cough, productive cough, wheezing, exercise intolerance, respiratory infections, bronchiectasis, chronic infections, nasal polyps, hemoptysis, pneumothorax, respiratory failure, dyspnea, chest pain, hemoptysis, pneumothorax, pulmonary vascular remodeling, pulmonary fibrosis, pulmonary vascular endothelial dysfunction, hypoxia due to chronic pulmonary injury, hypoxic pulmonary vasoconstriction, inflammation, smooth muscle hypertrophy, and right ventricular hypertrophy.

In certain aspects, the disclosure provides a method of treating pulmonary hypertension associated with obstructive lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In certain aspects, the disclosure provides a method of treating, preventing, or reducing the progression rate and/or severity of one or more complications of pulmonary hypertension associated with obstructive lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In some embodiments, the obstructive lung disease is selected from the group consisting of chronic obstructive pulmonary disease (COPD), cystic fibrosis, asthma, emphysema, lymphangioleiomyomatosis, and chronic bronchitis. In some embodiments, the one or more complications of pulmonary hypertension associated with obstructive lung disease is selected from the group consisting of increased need for supplemental oxygen, reduced mobility, and decreased survival.

In certain aspects, the disclosure provides a method of treating pulmonary hypertension associated with restrictive lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In certain aspects, the disclosure provides a method of treating, preventing, or reducing the progression rate and/or severity of one or more complications of pulmonary hypertension associated with restrictive lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In some embodiments, the restrictive lung disease is selected from the group consisting of pulmonary fibrosis, interstitial lung disease, sarcoidosis, idiopathic pulmonary fibrosis, pneumoconiosis, obesity, scoliosis, myasthenia gravis, and pleural effusion. In some embodiments, the one or more complications of pulmonary hypertension associated with restrictive lung disease is selected from the group consisting of shortness of breath with exertion, shortness of breath during rest, shortness of breath with minimal activity, cough, dry cough, productive cough, chronic cough, fatigue, weight loss, anxiety, depression, and fibrosis.

In certain aspects, the disclosure provides a method of treating pulmonary hypertension associated with combined obstructive and restrictive lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In some embodiments, the combined obstructive and restrictive lung disease is a pulmonary parenchymal disorder. In some embodiments, the pulmonary parenchymal disorder is selected from the group consisting of: sarcoidosis, COPD and ILD, COPD and idiopathic pulmonary fibrosis, pneumoconiosis, ILD, Langerhans cell histiocytosis, IPF, pulmonary alveolar proteinosis, lymphangioleiomyomatosis, and bronchiolitis obliterans syndrome. In some embodiments, the pneumoconiosis is selected from the group consisting of silicosis, coal worker's lung, and berylliosis. In some embodiments, the ILD is associated with systemic lupus erythematosus, rheumatoid arthritis, connective tissue disease, interstitial pneumonitis, constrictive bronchiolitis, or cryptogenic organizing pneumonia.

In some embodiments, the combined obstructive and restrictive lung disease is a combination of pulmonary parenchymal disorder and a non-pulmonary disease. In some embodiments, the combination of pulmonary parenchymal disorder and non-pulmonary disease is selected from the group consisting of: COPD and other non-parenchymal diseases, CHF and other non-pulmonary diseases, asthma and other disorder, ILD and obesity, ILD and CHF, and lung hypoplasia and scoliosis.

In some embodiments, the COPD and other non-parenchymal disease is selected from the group consisting of COPD and congestive heart failure (CHF), COPD and obesity, COPD and thoracic surgery, COPD and diaphragm paralysis, COPD and scoliosis, and COPD and pleurodesis. In some embodiments, the CHF and other non-pulmonary disease is selected from the group consisting of CHF and scoliosis, CHF and lung resection, and CHF and obesity. In some embodiments, the asthma and other disorder are selected from the group consisting of asthma and obesity, asthma and lung resection, asthma and radiation fibrosis, asthma and trapped lung, and asthma and CHF.

In certain aspects, the disclosure provides a method of treating pulmonary hypertension associated with interstitial lung disease (ILD), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the method reduces the right ventricular systolic pressure (RVSP) by at least 10%.

In certain aspects, the disclosure provides a method of treating, preventing, or reducing the progression rate and/or severity of one or more complications of pulmonary hypertension associated with interstitial lung disease (ILD), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, %%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In some embodiments, the ILD is associated with a condition selected from the group consisting of a connective tissue disease, sarcoidosis, vascular destruction due to progressive parenchymal fibrosis, vascular inflammation, perivascular fibrosis, thrombotic angiopathy, and endothelial dysfunction. In some embodiments, the connective tissue disease is selected from the group consisting of systemic sclerosis, rheumatoid arthritis, polymositis, dermatomyositis, and Sjogren syndrome.

In certain aspects, the disclosure provides a method of treating pulmonary hypertension associated with chronic obstructive pulmonary disease (COPD), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In certain aspects, the disclosure provides a method of treating, preventing, or reducing the progression rate and/or severity of one or more complications of pulmonary hypertension associated with chronic obstructive pulmonary disease (COPD), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In some embodiments, the one or more complications of pulmonary hypertension associated with chronic obstructive pulmonary disease (COPD) is selected from the group consisting of wheezing, productive cough, frequent cough, tightness in the chest, shortness of breath without physical activity, shortness of breath with physical activity, respiratory infection, weight loss, weakness in the muscles of the lower extremities, swelling in the lower extremities, and heart disease. In some embodiments, the patient has COPD with Gold grade 1, Gold grade 2, Gold grade 3, or Gold grade 4 as recognized by the Global Initiative for Chronic Obstructive Lung Disease. In some embodiments, the patient has Group A COPD, Group B COPD, Group C COPD, or Group D COPD. In some embodiments, the patients has COPD selected from the group consisting of: Stage 1, Stage 2, Stage 3, and Stage 4. In some embodiments, the patient has alpha-1-antityrypsin deficiency.

In certain aspects, the disclosure provides a method of treating pulmonary hypertension associated with combined pulmonary fibrosis and emphysema (CPFE), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In certain aspects, the disclosure provides a method of treating, preventing, or reducing the progression rate and/or severity of one or more complications of pulmonary hypertension associated with combined pulmonary fibrosis and emphysema (CPFE), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In certain aspects, the disclosure provides a method of treating pulmonary hypertension associated with fibrotic idiopathic interstitial pneumonia (IIP), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In certain aspects, the disclosure provides a method of treating, preventing, or reducing the progression rate and/or severity of one or more complications of pulmonary hypertension associated with fibrotic idiopathic interstitial pneumonia (IIP), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the patient has one or more diagnostic parameters selected from the group consisting of a high fibrotic score and a low diffusing capacity for carbon monoxide (DL_{CO}).

In certain aspects, the disclosure provides a method of treating pulmonary hypertension associated with idiopathic pulmonary fibrosis (IPF), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In certain aspects, the disclosure provides a method of treating, preventing, or reducing the progression rate and/or severity of one or more complications of pulmonary hypertension associated with idiopathic pulmonary fibrosis (IPF), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the one or more complications of pulmonary hypertension associated with idiopathic pulmonary fibrosis (IPF) is selected from the group consisting of increased need for supplemental oxygen, reduced mobility, and decreased survival.

In certain aspects, the disclosure provides a method of treating pulmonary hypertension associated with non-idiopathic pulmonary fibrosis interstitial lung disease (non-IPF ILD), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In certain aspects, the disclosure provides a method of treating, preventing, or reducing the progression rate and/or severity of one or more complications of pulmonary hypertension associated with non-idiopathic pulmonary fibrosis interstitial lung disease (non-IPF ILD), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the non-IPF ILD is selected from the group consisting of smoking-related ILD, hypersensitivity pneumonitis related ILD, connective tissue-related ILD, occupation-related ILD, and medication-induced ILD. In some embodiments, the one or more complications of pulmonary hypertension associated with non-IPF ILD is selected from the group consisting of increased need for supplemental oxygen, reduced mobility, and decreased survival.

In certain aspects, the disclosure provides a method of treating pulmonary hypertension associated with nonspecific interstitial pneumonia (NSIP), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In certain aspects, the disclosure provides a method of treating, preventing, or reducing the progression rate and/or severity of one or more complications of pulmonary hypertension associated with nonspecific interstitial pneumonia (NSIP), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1.

In some embodiments, the patient has a right ventricular systolic pressure (RVSP) of greater than 35 mmHg prior to treatment. In some embodiments, the method decreases the RVSP in the patient. In some embodiments, the method reduces the RVSP in the patient by at least 10% 15%, 20%, 25%, 30%, 35%, 40%, 45%, or at least 50%. In some embodiments, the method reduces the RVSP in the patient to less than 25 mmHg.

In some embodiments, the patient has a pulmonary artery systolic pressure (PASP) of greater than 25 mmHg prior to treatment. In some embodiments, the patient has a PASP of at least 35 mmHg, 40 mmHg, 45 mmHg, 50 mmHg, 55 mmHg, or 60 mmHg prior to treatment.

In some embodiments, the method decreases the PASP in the patient. In some embodiments, the method reduces the PASP in the patient by at least 10%, at least 15%, at least 20%, at least 25%, at least 30%, at least 35%, at least 40%, at least 45%, or at least 50%. In some embodiments, the method reduces the PASP in the patient by at least 5 mmHg (e.g., at least 5 mmHg, 10 mmHg, 15 mmHg, 20 mmHg, or 25 mmHg). In some embodiments, the method reduces the PASP in the patient to less than 25 mmHg. In some embodiments, the method reduces the PASP in the patient to less than 20 mmHg.

In some embodiments, the patient has a pulmonary vascular resistance (PVR) greater than or equal to 3 Wood Units prior to treatment. In some embodiments, the method decreases the PVR in the patient. In some embodiments, the method reduces the PVR in the patient by at least, 10%, 15%, 20%, 25%, 30%, 35%, 40%, 45%, or 50%. In some embodiments, the method reduces the PVR to less than 3 Woods Units.

In some embodiments, the patient has a mean pulmonary artery pressure (mPAP) prior to treatment selected from the group consisting of an mPAP of at least 17 mmHg, an mPAP of at least 20 mmHg, an mPAP of at least 25 mmHg, an mPAP of at least 30 mmHg, an mPAP of at least 35 mmHg, an mPAP of at least 40 mmHg, an mPAP of at least 45 mmHg, and an mPAP of at least 50 mmHg. In some embodiments, the patient has an mPAP prior to treatment between 21-24 mmHg and a PVR prior to treatment of at least 3 Wood Units.

In some embodiments, the patient has an mPAP prior to treatment of greater than 25 mmHg with a Cardiac Index (CI) of less than 2.0 L/min/m². In some embodiments, the patient has an mPAP prior to treatment of greater than 25 mmHg with a CI of less than 2.5 L/min/m².

In some embodiments, the method reduces the mPAP in the patient by at least 10%, 15%, at least 20%, at least 25%, at least 30%, at least 35%, at least 40%, at least 45%, or at least 50%. In some embodiments, the method reduces the mPAP by at least 3 mmHg, 5, 7, 10, 12, 15, 20, or 25 mm Hg in the patient. In some embodiments, the method reduces the mPAP to a value selected from the group consisting of less than 17 mmHg, less than 20 mmHg, less than 25 mmHg, and less than 30 mmHg.

In some embodiments, the patient has a mean right atrial pressure (mRAP) prior to treatment selected from the group consisting of an mRAP of at least 5 mmHg, an mRAP of at least 6 mmHg, an mRAP of at least 8 mmHg, an mRAP of at least 10 mmHg, an mRAP of at least 12 mmHg, an mRAP of at least 14 mmHg, and an mRAP of at least 16 mmHg. In some embodiments, the method improves the mRAP in the patient. In some embodiments, the improvement in the mRAP is a reduction in the mRAP. In some embodiments, the method reduces the mRAP in the patient by at least, 10%, 15%, 20%, 25%, 30%, 35%, 40%, 45%, or 50%. In some embodiments, the method reduces the mRAP by at least 1, 2, 3, 4, 5, 6, 7, 8, 9, 10, 11, 12, 13, 14, or 15 mm Hg in the patient.

In some embodiments, the patient has a cardiac output of less than 4 L/min prior to treatment. In some embodiments, the method increases the cardiac output in the patient by at least 10%, 15%, 20%, 25%, 30%, 35%, 40%, 45%, or 50%. In some embodiments, the method increases the cardiac output in the patient by at least 0.5 L/min, 1, 1.5, 2, 2.5, 3, 3.5, or 4 L/min in the patient. In some embodiments, the method increases the cardiac output in the patient to at least 4 L/min.

In some embodiments, the patient has a cardiac index (CI) of less than 2.5 L/min/m², 2.0, 1.5, or 1 L/min/m² prior to treatment. In some embodiments, the method increases the CI in the patient by at least 10%, 15%, 20%, 25%, 30%, 35%, 40%, 45%, or 50%. In some embodiments, the method increases the CI in the patient by at least 0.2 L/min/m², 0.4, 0.6, 0.8, 1, 1.2, 1.4, 1.6, 1.8, or 2 L/min/m² in the patient. In some embodiments, the method increases the CI in the patient to at least 2.5 L/min/m².

In some embodiments, the method increases exercise capacity of the patient. In some embodiments, the patient has a Borg dyspnea index (BDI) at least 0.5 index points, 1, 1.5, 2, 2.5, 3, 3.5, 4, 4.5, 5, 5.5, 6, 6.5, 7, 7.5, 8, 8.5, 9, 9.5, or 10 index points prior to treatment. In some embodiments, the method reduces the patient's BDI. In some embodiments, the method reduces the patient's BDI by at least 0.5, 1, 1.5, 2, 2.5, 3, 3.5, 4, 4.5, 5, 5.5, 6, 6.5, 7, 7.5, 8, 8.5, 9, 9.5, or 10 index points.

In some embodiments, the patient has a 6-minute walk distance (6MWD) of less than 550 meters, 500, 450, 440, 400, 380, 350, 300, 250, 200, or 150 meters prior to treatment. In some embodiments, the method increases the patient's 6MWD by at least 10 meters, 15, 20, 25, 30, 35, 40, 45, 50, 55, 60, 65, 70, 75, 80, 85, 90, 95, 100, 125, 150, 175, 200, 250, 300, or 400 meters.

In some embodiments, the method prevents or reduces pulmonary hypertension Functional Class progression as recognized by the World Health Organization (WHO). In some embodiments, the method prevents or reduces pulmonary hypertension Functional Class progression from Functional Class I to Class II pulmonary hypertension as recognized by the WHO. In some embodiments, the method prevents or reduces pulmonary hypertension Functional Class progression from Functional Class II to Class III pulmonary hypertension as recognized by the WHO. In some embodiments, the method prevents or reduces pulmonary hypertension Functional Class progression from Functional Class III to Class IV pulmonary hypertension as recognized by the WHO. In some embodiments, the method promotes or increases pulmonary hypertension Functional Class regression as recognized by the WHO. In some embodiments, the method promotes or increases pulmonary hypertension Functional Class regression from Class IV to Class III pulmonary hypertension as recognized by the WHO. In some embodiments, the method promotes or increases pulmonary hypertension Functional Class regression from Class III to Class II pulmonary hypertension as recognized by the WHO. In some embodiments, the method promotes or increases pulmonary hypertension Functional Class regression from Class II to Class I pulmonary hypertension as recognized by the WHO.

In some embodiments, the patient has elevated NT-proBNP levels as compared to a healthy patient prior to treatment. In some embodiments, the patient has normal NT-proBNP levels. In some embodiments, the patient has a NT-proBNP level of at least 100 pg/mL, 150, 200, 300, 400, 500, 1000, 3000, 5000, 10,000, 15,000, or 20,000 pg/mL prior to treatment. In some embodiments, the method decreases NT-proBNP levels in the patient. In some embodiments, the method decreases NT-proBNP levels in the patient by at least 10%, 15%, 20%, 25%, 30%, 35%, 40%, 45%, 50%, 55%, 60%, 65%, 70%, 75%, or 80%. In some embodiments, the method decreases NT-proBNP levels in the patient by at least 30%. In some embodiments, the method decreases NT-proBNP levels to normal levels. In some embodiments, the normal level of NT-proBNP is <100 pg/ml.

In some embodiments, the patient has elevated brain natriuretic peptide (BNP) levels as compared to a healthy patient prior to treatment. In some embodiments, the patient has normal BNP levels prior to treatment. In some embodiments, the patient has a BNP level of at least 100 pg/mL, 150, 200, 300, 400, 500, 1000, 3000, 5000, 10,000, 15,000, or 20,000 pg/mL prior to treatment. In some embodiments, the method decreases BNP levels in the patient by at least 10%, 15%, 20%, 25%, 30%, 35%, 40%, 45%, 50%, 55%, 60%, 65%, 70%, 75%, or at least 80%. In some embodiments, the method decreases BNP levels to normal levels (i.e., <100 pg/ml).

In some embodiments, the patient has a diastolic pressure gradient (DPG) of greater than 7 mmHg prior to treatment. In some embodiments, the patient has a DPG of at least 7 mmHg (e.g., at least 7, 10, 15, 20, 25, 30, 35, 40, 45, or 50 mmHg) prior to treatment. In some embodiments, the method decreases the DPG in the patient. In some embodiments, the method reduces the DPG in the patient by at least 10% (e.g., 10%, 15%, 20%, 25%, 30%, 35%, 40%, 45%, or at least 50%). In some embodiments, the method reduces the DPG in the patient to less than 7 mmHg.

In some embodiments, the method increases the patient's quality of life by at least 1% (e.g., 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the patient's quality of life is measured using the Cambridge Pulmonary Hypertension Outcome Review (CAMPHOR).

In some embodiments, the patient has pulmonary fibrosis. In some embodiments, the method decreases the pulmonary fibrosis in the patient. In some embodiments, the method reduces the pulmonary fibrosis in the patient by at least 10%, 15%, 20%, 25%, 30%, 35%, 40%, 45%, or 50%.

In some embodiments, the patient has a diffusing capacity of carbon monoxide (DL_{CO}) less than 60%, 55%, 50%, 45%, 40%, 35%, 30%, 25%, or 20% prior to treatment. In some embodiments, the method increases the DL_{CO} in the patient. In some embodiments, the method increases the DL_{CO} in the patient by at least 5%, 10%, 15%, 20%, 25%, 30%, 35%, 40%, 45%, or 50%. In some embodiments, the method increases the DL_{CO} to at least 40%, 45%, 50%, 55%, 60%, or 65%.

In some embodiments, the patient has a carbon monoxide transfer coefficient (K_{CO}) less than 60% of predicted values, less than 55% of predicted values, less than 50% of predicted values, less than 45% of predicted values, less than 40% of predicted values, less than 35% of predicted values, less than 30% of predicted values, less than 25% of predicted values, or less than 20% of predicted values. In some embodiments, the method increases the K_{CO} in the patient. In some embodiments, the method increases the K_{CO} in the patient by at least 5%, 10%, 15%, 20%, 25%, 30%, 35%, 40%, 45%, or 50%. In some embodiments, the method increases the K_{CO} to at least 40%, 45%, 50%, 55%, 60%, or 65%.

In some embodiments, the patient has a composite physiologic index (CPI) greater than 15, 20, 25, 30, 35, 40, 45, 50, 55, 60, 65, 70, 75 and 80 prior to treatment. In some embodiments, the method decreases the CPI in the patient. In some embodiments, the method decreases the CPI in the patient by at least 10%, 15%, 20%, 25%, 30%, 35%, 40%, 45%, or 50%. In some embodiments, the method decreases the CPI to less than 70, 65, 60, 55, 50, 45, 40, 35, 30, 25, 20, 15, 10, or 5.

In some embodiments, the patient has an arterial oxygen saturation of less than 95%, 90%, 85%, 80%, 75%, 70%, 65%, 60%, 55%, 50%, 45%, 40%, 35%, or 30% prior to treatment.

In some embodiments, the method increases the arterial oxygen saturation in the patient. In some embodiments, the method increases the arterial oxygen saturation in the patient by at least 5%, 10%, 15%, 20%, 25%, 30%, 35%, 40%, 45%, or at least 50%. In some embodiments, the method increases the arterial oxygen saturation to at least 85%, 90%, or 95%. In some embodiments, the arterial oxygen saturation is measured at rest.

In some embodiments, the patient has a TAPSE of less than 20 mm, 18, 16, 14, or 12 mm. In some embodiments, the method increases the TAPSE to at least 20 mm, 22, 24, 26, 28, or 30 mm.

In some embodiments, the patient has a forced expiratory volume in one second (FEV₁) selected from the group consisting of greater than 70%, between 60% to 69%, between 50% to 59%, between 35% to 49%, and less than 35% prior to treatment. In some embodiments, the method increases the FEV₁ in the patient. In some embodiments, the method increases the FEV₁ in the patient by at least 5%, 10%, 15%, 20%, 25%, 30%, 35%, 40%, 45%, or 50%. In some embodiments, the method increases the FEV₁ to at least 60%, 65%, 70%, 75%, 80%, 85%, 90%, or 95%.

In some embodiments, the patient has a forced vital capacity (FVC) selected from the group consisting of greater than 80%, greater than 70%, between 60% to 69%, between 50% to 59%, between 35% to 49%, and less than 35% prior to treatment. In some embodiments, the method increases the FVC in the patient. In some embodiments, the method increases the FVC in the patient by at least 5%, 10%, 15%, 20%, 25%, 30%, 35%, 40%, 45%, or 50%. In some embodiments, the method increases the FVC to at least 60%, 65%, 70%, 75%, 80%, 85%, 90%, or 95%.

In some embodiments, the method improves right ventricular function in the patient. In some embodiments, the improvement in right ventricular function is due to an increase in right ventricular fractional area change. In some embodiments, the improvement in right ventricular function is due to a decrease in right ventricular hypertrophy. In some embodiments, the improvement in right ventricular function is due to an increase in ejection fraction. In some embodiments, the improvement in right ventricular function is due to an increase in right ventricular fractional area change and ejection fraction. In some embodiments, the method decreases right ventricular hypertrophy in the patient. In some embodiments, the method decreases right ventricular hypertrophy in the patient by at least 10%, 15%, 20%, 25%, 30%, 35%, 40%, 45%, or 50%.

In some embodiments, the method decreases smooth muscle hypertrophy in the patient. In some embodiments, the method decreases smooth muscle hypertrophy in the patient by at least 10%, 15%, 20%, 25%, 30%, 35%, 40%, 45%, or 50%.

In some embodiments, the method reduces the risk of death. In some embodiments, the method reduces the risk of death associated with pulmonary arterial hypertension by at least 10%, 15%, 20%, 25%, 30%, 35%, 40%, 45%, or 50%.

In some embodiments, the method increases transplant free survival in the patient. In some embodiments, the method increases transplant free survival in the patient by at least 10%, 15%, 20%, 25%, 30%, 35%, 40%, 45%, or 50%.

In some embodiments, the method treats one or more comorbidities of pulmonary hypertension associated with lung disease. In some embodiments, the one or more comorbidities of pulmonary hypertension associated with lung disease are selected from the group consisting of systemic hypertension, decreased renal function, diabetes mellitus, hyperlipidemia, obesity, coronary artery disease (CAD), obstructive sleep apnea, pulmonary embolism, heart failure, atrial fibrillation and anemia.

In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the sequence of amino acids corresponding to residues 30-110 of SEQ ID NO: 1. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence SEQ ID NO: 2. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3.

In some embodiments, the ActRII polypeptide is a fusion protein further comprising an Fc domain of an immunoglobulin. In some embodiments, the Fc domain of the immunoglobulin is an Fc domain of an IgG1 immunoglobulin. In some embodiments, the Fc fusion protein further comprises a linker domain positioned between the ActRII polypeptide domain and the Fc domain of the immunoglobulin. In some embodiments, the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21).

In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 23. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 41.

In some embodiments, the polypeptide comprises an amino acid sequence that is at least 90% identical to an amino acid sequence corresponding to residues 30-110 of SEQ ID NO: 1, wherein the polypeptide binds to activin and/or GDF11. In some embodiments, the polypeptide comprises an amino acid sequence that is at least 90% identical to an amino acid sequence corresponding to residues 21-135 of SEQ ID NO: 1, wherein the polypeptide binds to activin and/or GDF11.

In some embodiments, the polypeptide is lyophilized. In some embodiments, the polypeptide is soluble. In some embodiments, the polypeptide is administered using subcutaneous injection. In some embodiments, the polypeptide is administered about every 3 weeks. In some embodiments, the polypeptide is administered about every 4 weeks.

In some embodiments, the polypeptide is part of a homodimer protein complex. In some embodiments, the polypeptide is glycosylated. In some embodiments, the polypeptide has a glycosylation pattern obtainable by expression in a Chinese hamster ovary cell.

In some embodiments, the ActRII polypeptide binds to one or more ligands selected from the group consisting of: activin A, activin B, and GDF11. In some embodiments, the ActRII polypeptide further binds to one or more ligands selected from the group consisting of: BMP10, GDF8, and BMP6.

In some embodiments, the ActRII polypeptide is administered at a dose from 0.1 mg/kg to 2.0 mg/kg. In some embodiments, the ActRII polypeptide is administered at a dose of 0.3 mg/kg. In some embodiments, the ActRII polypeptide is administered at a dose of 0.7 mg/kg.

In some embodiments, the methods disclosed herein comprise further administering to the patient an additional active agent and/or supportive therapy. In some embodiments, the additional active agent and/or supportive therapy is selected from the group consisting of: beta-blockers, angiotensin-converting enzyme inhibitors (ACE inhibitors), angiotensin receptor blockers (ARBs), diuretic agents, lipid-lowering medications, endothelin blockers, PDE5 inhibitors, and prostacyclins. In some embodiments, the additional active agent and/or supportive therapy is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); digoxin, diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-uB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SC≥015; SZC017; PEGylated derivatives of oleanolic acid; 3-O-(beta-D-glucopyranosyl) oleanolic acid; 3-O-[beta-D-glucopyranosyl-(1- ->3)-beta-D-glucopyranosyl] oleanolic acid; 3-O-[beta-D-glucopyranosyl-(1- ->2)-beta-D-glucopyranosyl] oleanolic acid; 3-O-[beta-D-glucopyranosyl-(1- ->3)-beta-D-glucopyranosyl]oleanolic acid 28-O-beta-D-glucopyranosyl ester, 3-O-[beta-D-glucopyranosyl-(1- ->2)-beta-D-glucopyranosyl] oleanolic acid 28-O-beta-D-glucopyranosyl ester; 3-O-[a-L-rhamnopyranosyl-(1- ->3)-beta-D-glucuronopyranosyl] oleanolic acid; 3-O-[alpha-L-rhamnopyranosyl-(1- ->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate); a left ventricular assist device (LVAD), oxygen therapy, and lung and/or heart transplantation.

In some embodiments, the patient has been treated with one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil. In some embodiments, the method further comprises administration of one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil.

In some embodiments, the patient has been treated with one or more vasodilators prior to administration of the polypeptide. In some embodiments, the method further comprises administration of one or more vasodilators. In some embodiments, the one or more vasodilators is selected from the group consisting of prostacyclin, epoprostenol, and sildenafil. In some embodiments, the vasodilator is prostacyclin.

In some embodiments, the patient has been receiving one or more therapies for pulmonary hypertension associated with lung disease. In some embodiments, the one or more therapies for pulmonary hypertension associated with lung disease is selected from the group consisting of: treprostinil, pirfenidone, nintedanib, prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SC≥015; SZC017; PEGylated derivatives of oleanolic acid; 3-O-(beta-D-glucopyranosyl) oleanolic acid; 3-O-[beta-D-glucopyranosyl-(1- ->3)-beta-D-glucopyranosyl] oleanolic acid; 3-O-[beta-D-glucopyranosyl-(1- ->2)-beta-D-glucopyranosyl] oleanolic acid; 3-O-[beta-D-glucopyranosyl-(1- ->3)-beta-D-glucopyranosyl] oleanolic acid 28-O-beta-D-glucopyranosyl ester, 3-O-[beta-D-glucopyranosyl-(1- ->2)-beta-D-glucopyranosyl] oleanolic acid 28-O-beta-D-glucopyranosyl ester; 3-O-[a-L-rhamnopyranosyl-(1- ->3)-beta-D-glucuronopyranosyl] oleanolic acid; 3-O-[alpha-L-rhamnopyranosyl-(1- ->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate); a left ventricular assist device (LVAD), oxygen therapy, and lung and/or heart transplantation.

In some embodiments, the ActRII polypeptide is administered to the patient about every week, about every two weeks, about every three weeks, or about every four weeks. In some embodiments, the ActRII polypeptide is administered to the patient every three weeks.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1 shows an alignment of extracellular domains of human ActRIIB (SEQ ID NO: 31) and human ActRIIA (SEQ ID NO: 2) with the residues that are deduced herein, based on composite analysis of multiple ActRIIB and ActRIIA crystal structures, to directly contact ligand indicated with boxes.

FIG. 2 shows a multiple sequence alignment of various vertebrate ActRIIA proteins and human ActRIIA (SEQ ID NOs: 6-10 and 36-38).

FIG. 3 shows multiple sequence alignment of Fc domains from human IgG isotypes using Clustal 2.1. Hinge regions are indicated by dotted underline. Double underline indicates examples of positions engineered in IgG1 Fc (SEQ ID NO: 32) to promote asymmetric chain pairing and the corresponding positions with respect to other isotypes IgG2 (SEQ ID NO: 33), IgG3 (SEQ ID NO: 34) and IgG4 (SEQ ID NO: 35).

FIGS. 4A and 4B show the purification of ActRIIA-hFc expressed in CHO cells. The protein purifies as a single, well-defined peak as visualized by sizing column (FIG. 4A) and Coomassie stained SDS-PAGE (FIG. 4B) (left lane: molecular weight standards; right lane: ActRIIA-hFc).

FIGS. 5A and 5B show the binding of ActRIIA-hFc to activin (FIG. 5A) and GDF-11 (FIG. 5B), as measured by Biacore™ assay.

FIGS. 6A-6D show the effect of ActRIIA-mFc treatment of pulmonary hypertension and RV hypertrophy in Bleo-MCT PH-ILD rat model. Rx: ActRIIA-mFc s.c. 5mpk, BIW; Bleo: bleomycin; MCT: Monocrotaline.

FIGS. 7A-7C show the effect of ActRIIA-mFc treatment of pulmonary hypertension and RV hypertrophy in Bleo/Su/Hx PH-ILD rat model. Rx: ActRIIA-mFc s.c. 5mpk, BIW; Bleo: bleomycin; MCT: Monocrotaline.

FIGS. 8A-8C show the effect of ActRIIA-mFc treatment on Group 3 pulmonary hypertension in LPS induced COPD rat model.

### DETAILED DESCRIPTION

### 1. Overview

The present disclosure relates to compositions and methods of treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) comprising administering to a patient in need thereof an effective amount of an ActRII polypeptide as described herein. In certain embodiments, the present disclosure provides methods of treating or preventing pulmonary hypertension associated with lung disease (e.g., pulmonary hypertension associated with COPD, ILD, or CPFE) in an individual in need thereof through administering to the individual a therapeutically effective amount of an ActRII polypeptide as described herein.

Most lung diseases can be categorized as either obstructive or restrictive. Lung diseases that are characterized by both obstruction and restriction occur infrequently and are commonly caused by a combination of pulmonary parenchymal and non-pulmonary disorders. Obstructive lung diseases (e.g., COPD, chronic bronchitis, asthma, bronchiectasis, bronchiolitis, and cystic fibrosis) are characterized by an obstruction in the air passages and defined by exhalation that is slower and shallower than in a healthy individual. Restrictive lung diseases (e.g., adult respiratory distress syndrome (ARDS), pneumoconioses, pneumonia, eosinophilic pneumonia, tuberculosis, sarcoidosis, pulmonary fibrosis and idiopathic pulmonary fibrosis, pleural effusion, and pleurisy) are characterized by a reduced total lung capacity and defined by inhalation that fill the lungs far less than what is expected in a healthy individual. One of the prominent complications of lung disease is pulmonary hypertension. Pulmonary hypertension associated with lung disease (e.g., pulmonary hypertension associated with COPD, ILD, or CPFE) [World Health Organization Group 3 PH] is a progressive disease marked by inflammation and irreversible scarring of the lung tissue. Chronic lung disease is the second leading cause for pulmonary hypertension. The mortality rates for patients with pulmonary hypertension associated with lung disease (e.g., pulmonary hypertension associated with COPD, ILD, or CPFE) is the highest reported of any of the five diagnostic groups of pulmonary hypertension. Currently there is only one U.S. Food and Drug Administration-approved treatment for pulmonary hypertension associated with lung disease, treprostinil, which is also approved for the treatment of pulmonary arterial hypertension (PAH; WHO Group 1 pulmonary hypertension). All other treatments in clinical practice of pulmonary hypertension associated with lung disease are based on management of the underlying lung disease, as well as off-label use of certain treatments approved for pulmonary arterial hypertension (PAH) [World Health Organization (WHO) Group 1 PH].

Pulmonary hypertension associated with lung disease (e.g., pulmonary hypertension associated with COPD, ILD, or CPFE) can be definitively diagnosed using right heart catheterization, however echocardiography remains a good screening and monitoring tool for patients thought to be at risk. Echocardiography is used to detect elevated pulmonary artery systolic pressures (ePASP) as well as altered right-sided ventricle structure or dysfunction and evidence of left-sided heart disease. Other assessments and/or tools (e.g., the 6-min walk test (6MWT), computed tomography (CT) scans, and pulmonary function tests). Despite being the only definitive test for pulmonary hypertension associated with lung disease, right heart catheterization is not required for every patient suspected of having this disease. However, in cases of suspected moderate or severe pulmonary hypertension, as well as suspected alternate etiologies for pulmonary hypertension, right heart catheterization is recommended.

In certain aspects, the disclosure relates to methods related to treating, preventing, or reducing the progression rate and/or severity of one or more complications of pulmonary hypertension associated with lung disease (e.g., an obstructive lung disease, a restrictive lung disease, or a combined obstructive and restrictive lung disease), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1. The one or more complications of pulmonary hypertension associated with lung disease is selected from the group consisting of persistent cough, productive cough, wheezing, exercise intolerance, respiratory infections, bronchiectasis, chronic infections, nasal polyps, hemoptysis, pneumothorax, respiratory failure, dyspnea, chest pain, hemoptysis, pneumothorax, pulmonary vascular remodeling, pulmonary fibrosis, pulmonary vascular endothelial dysfunction, hypoxia due to chronic pulmonary injury, hypoxic pulmonary vasoconstriction, inflammation, smooth muscle hypertrophy, and right ventricular hypertrophy.

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is ±10%. Alternatively, and particularly in biological systems, the terms “about” and “approximately” may mean values that are within an order of magnitude, preferably ≤55-fold and more preferably ≤2-fold of a given value.

Numeric ranges disclosed herein are inclusive of the numbers defining the ranges. The term “between” as used in the present application is inclusive of the numbers defining the ranges. Moreover, all ranges disclosed herein are to be understood to encompass any and all subranges subsumed therein. For example, a stated range of “1 to 10” or “between 1 to 10” should be considered to include any and all subranges between and inclusive of the minimum value of 1 or more, e.g., 1 to 6.1, and ending with a maximum value of 10 or less, e.g., 5.5 to 10.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. ActRII Polypeptides

In certain aspects, the disclosure relates to ActRII polypeptides and uses thereof (e.g., of treating, preventing, or reducing the progression rate and/or severity of pulmonary hypertension associated with lung disease (e.g.e.g., pulmonary hypertension associated with COPD, ILD, or (CPFE) or one or more complications of pulmonary hypertension associated with lung disease (e.g., pulmonary hypertension associated with COPD, ILD, or CPFE). As used herein, the term “ActRII” refers to the family of type II activin receptors. This family includes activin receptor type IIA (ActRIIA) and activin receptor type IIB (ActRIIB).

In certain embodiments, the present disclosure relates to ActRII polypeptides having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence as set forth in anyone of SEQ ID NOs: 1,2,3, 23, 27, 30, and 41. As used herein, the term “ActRII” refers to a family of activin receptor type IIA (ActRIIA) proteins, a family of activin receptor type IIB (ActRIIB) proteins, or combinations and/or variants thereof. The ActRII polypeptides can be derived from any species and include variants derived from such ActRII proteins by mutagenesis or other modification. Reference to ActRII herein is understood to be a reference to any one of the currently identified forms. Members of the ActRII family are generally transmembrane proteins, composed of a ligand-binding extracellular domain comprising a cysteine-rich region, a transmembrane domain, and a cytoplasmic domain with predicted serine/threonine kinase activity.

The term ActRII polypeptide includes polypeptides comprising any naturally occurring polypeptide of an ActRII family member as well as any variants thereof (including mutants, fragments, fusions, and peptidomimetic forms) that retain a useful activity. Numbering of amino acids for all ActRII-related polypeptides described herein is based on the numbering of the human ActRII precursor protein sequence provided below (SEQ ID NO: 1), unless specifically designated otherwise.

The canonical human ActRII precursor protein sequence is as follows:

| (SEQ ID NO: 1) | |
|---|---|
| 1 | MGAAAKLAFA VFLISCSSGA ILGRSETQEC LFFNANWEKD RT [symbol] QTGVEPC |
| 51 | YGDKDKRRHC FATWK [symbol] ISGS IEIVKQGCWL DDINCYDRTD CVEKKDSPEV |
| 101 | YFCCCEGNMC NEKFSYFPEM EVTQPTSNPV TPKPPYYNIL LYSLVPLMLI |
| 151 | AGIVICAFWV YRHHKMAYPP VLVPTQDPGP PPPSPLLGLK PLQLLEVKAR |
| 201 | GRFGCVWKAQ LLNEYVAVKI FPIQDKQSWQ NEYEVYSLPG MKHENILQFI |
| 251 | GAEKRGTSVD VDLWLITAFH EKGSLSDFLK ANVVSWNELC HIAETMARGL |
| 301 | AYLHEDIPGL KDGHKPAISH RDIKSKNVLL KNNLTACIAD FGLALKFEAG |
| 351 | KSAGDTHGQV GTRRYMAPEV LEGAINFQRD AFLRIDMYAM GLVLWELASR |
| 401 | CTAADGPVDE YMLPFEEEIG QHPSLEDMQE VVVHKKKRPV LRDYWQKHAG |
| 451 | MAMLCETIEE CWDHDAEARL SAGCVGERIT QMQRLTNIIT TEDIVTVVTM |
| 501 | VTNVDFPPKE SSL |

The signal peptide is indicated by a single underline; the extracellular domain is indicated in bold font; and the potential, endogenous N-linked glycosylation sites are indicated by a double underline.

A processed (mature) extracellular human ActRII polypeptide sequence is as follows:

(SEQ ID NO: 2)
ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPP

The C-terminal “tail” of the extracellular domain is indicated by single underline. The sequence with the “tail” deleted (a A15 sequence) is as follows:

(SEQ ID NO: 3)
ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EM

A nucleic acid sequence encoding human ActRII precursor protein is shown below (SEQ ID NO: 4), as follows nucleotides 159-1700 of Genbank Reference Sequence NM_001616.4. The signal sequence is underlined.

| (SEQ ID NO: 4) | |
|---|---|
| 1 | ATGGGAGCTG CTGCAAAGTT GGCGTTTGCC GTCTTTCTTA TCTCCTGTTC |
| 51 | TTCAGGTGCT ATACTTGGTA GATCAGAAAC TCAGGAGTGT CTTTTCTTTA |
| 101 | ATGCTAATTG GGAAAAAGAC AGAACCAATC AAACTGGTGT TGAACCGTGT |
| 151 | TATGGTGACA AAGATAAACG GCGGCATTGT TTTGCTACCT GGAAGAATAT |
| 201 | TTCTGGTTCC ATTGAAATAG TGAAACAAGG TTGTTGGCTG GATGATATCA |
| 251 | ACTGCTATGA CAGGACTGAT TGTGTAGAAA AAAAAGACAG CCCTGAAGTA |
| 301 | TATTTTTGTT GCTGTGAGGG CAATATGTGT AATGAAAAGT TTTCTTATTT |
| 351 | TCCGGAGATG GAAGTCACAC AGCCCACTTC AAATCCAGTT ACACCTAAGC |
| 401 | CACCCTATTA CAACATCCTG CTCTATTCCT TGGTGCCACT TATGTTAATT |
| 451 | GCGGGGATTG TCATTTGTGC ATTTTGGGTG TACAGGCATC ACAAGATGGC |
| 501 | CTACCCTCCT GTACTTGTTC CAACTCAAGA CCCAGGACCA CCCCCACCTT |
| 551 | CTCCATTACT AGGTTTGAAA CCACTGCAGT TATTAGAAGT GAAAGCAAGG |
| 601 | GGAAGATTTG GTTGTGTCTG GAAAGCCCAG TTGCTTAACG AATATGTGGC |
| 651 | TGTCAAAATA TTTCCAATAC AGGACAAACA GTCATGGCAA AATGAATACG |
| 701 | AAGTCTACAG TTTGCCTGGA ATGAAGCATG AGAACATATT ACAGTTCATT |
| 751 | GGTGCAGAAA AACGAGGCAC CAGTGTTGAT GTGGATCTTT GGCTGATCAC |
| 801 | AGCATTTCAT GAAAAGGGTT CACTATCAGA CTTTCTTAAG GCTAATGTGG |
| 851 | TCTCTTGGAA TGAACTGTGT CATATTGCAG AAACCATGGC TAGAGGATTG |
| 901 | GCATATTTAC ATGAGGATAT ACCTGGCCTA AAAGATGGCC ACAAACCTGC |
| 951 | CATATCTCAC AGGGACATCA AAAGTAAAAA TGTGCTGTTG AAAAACAACC |
| 1001 | TGACAGCTTG CATTGCTGAC TTTGGGTTGG CCTTAAAATT TGAGGCTGGC |
| 1051 | AAGTCTGCAG GCGATACCCA TGGACAGGTT GGTACCCGGA GGTACATGGC |
| 1101 | TCCAGAGGTA TTAGAGGGTG CTATAAACTT CCAAAGGGAT GCATTTTTGA |
| 1151 | GGATAGATAT GTATGCCATG GGATTAGTCC TATGGGAACT GGCTTCTCGC |
| 1201 | TGTACTGCTG CAGATGGACC TGTAGATGAA TACATGTTGC CATTTGAGGA |
| 1251 | GGAAATTGGC CAGCATCCAT CTCTTGAAGA CATGCAGGAA GTTGTTGTGC |
| 1301 | ATAAAAAAAA GAGGCCTGTT TTAAGAGATT ATTGGCAGAA ACATGCTGGA |
| 1351 | ATGGCAATGC TCTGTGAAAC CATTGAAGAA TGTTGGGATC ACGACGCAGA |
| 1401 | AGCCAGGTTA TCAGCTGGAT GTGTAGGTGA AAGAATTACC CAGATGCAGA |
| 1451 | GACTAACAAA TATTATTACC ACAGAGGACA TTGTAACAGT GGTCACAATG |
| 1501 | GTGACAAATG TTGACTTTCC TCCCAAAGAA TCTAGTCTA |

A nucleic acid sequence encoding processed soluble (extracellular) human ActRII polypeptide is as follows:

| (SEQ ID NO: 5) | |
|---|---|
| 1 | ATACTTGGTA GATCAGAAAC TCAGGAGTGT CTTTTCTTTA ATGCTAATTG |
| 51 | GGAAAAAGAC AGAACCAATC AAACTGGTGT TGAACCGTGT TATGGTGACA |
| 101 | AAGATAAACG GCGGCATTGT TTTGCTACCT GGAAGAATAT TTCTGGTTCC |
| 151 | ATTGAAATAG TGAAACAAGG TTGTTGGCTG GATGATATCA ACTGCTATGA |
| 201 | CAGGACTGAT TGTGTAGAAA AAAAAGACAG CCCTGAAGTA TATTTTTGTT |
| 251 | GCTGTGAGGG CAATATGTGT AATGAAAAGT TTTCTTATTT TCCGGAGATG |
| 301 | GAAGTCACAC AGCCCACTTC AAATCCAGTT ACACCTAAGC CACCC |

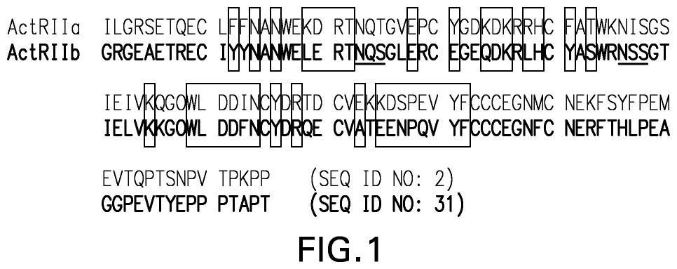

An alignment of the amino acid sequences of human ActRIIA extracellular domain and human ActRIIB extracellular domain are illustrated in FIG. 1. This alignment indicates amino acid residues within both receptors that are believed to directly contact ActRII ligands. For example, the composite ActRII structures indicated that the ActRIIA-ligand binding pocket is defined, in part, by residues F31, N33, N35, K38 through T41, E47, Y50, K53 through K55, R57, H58, F60, T62, K74, W78 through N83, Y85, R87, E92, and K94 through F10. At these positions, it is expected that conservative mutations will be tolerated.

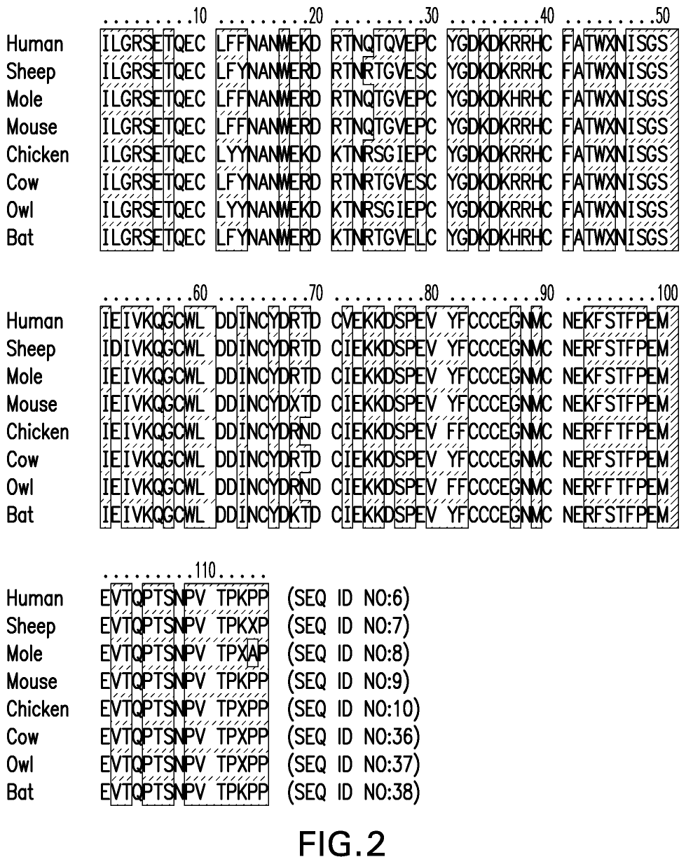

ActRII is well-conserved among vertebrates, with large stretches of the extracellular domain completely conserved. For example, FIG. 2 depicts a multi-sequence alignment of a human ActRIIA extracellular domain compared to various ActRIIA orthologs. Many of the ligands that bind to ActRIIA are also highly conserved. Accordingly, from these alignments, it is possible to predict key amino acid positions within the ligand-binding domain that are important for normal ActRII-ligand binding activities as well as to predict amino acid positions that are likely to be tolerant to substitution without significantly altering normal ActRII-ligand binding activities. Therefore, an active, human ActRII variant polypeptide useful in accordance with the presently disclosed methods may include one or more amino acids at corresponding positions from the sequence of another vertebrate ActRII, or may include a residue that is similar to that in the human or other vertebrate sequences.

Without meaning to be limiting, the following examples illustrate this approach to defining an active ActRII variant. As illustrated in FIG. 2, F13 in the human extracellular domain is Y in Ovis aries (SEQ ID NO: 7), Gallus gallus (SEQ ID NO: 10), Bos Taurus (SEQ ID NO: 36), Tyto alba (SEQ ID NO: 37), and Myotis davidii (SEQ ID NO: 38) ActRIIA, indicating that aromatic residues are tolerated at this position, including F, W, and Y. Q24 in the human extracellular domain is R in Bos Taurus ActRIIA, indicating that charged residues will be tolerated at this position, including D, R, K, H, and E. S95 in the human extracellular domain is F in Gallus gallus and Tyto alba ActRIIA, indicating that this site may be tolerant of a wide variety of changes, including polar residues, such as E, D, K, R, H, S, T, P, G, Y, and probably hydrophobic residue such as L, I, or F. E52 in the human extracellular domain is D in Ovis aries ActRIIA, indicating that acidic residues are tolerated at this position, including D and E. P29 in the human extracellular domain is relatively poorly conserved, appearing as S in Ovis aries ActRIIA and L in Myotis davidii ActRIIA, thus essentially any amino acid should be tolerated at this position.

Moreover, as discussed above, ActRII proteins have been characterized in the art in terms of structural/functional characteristics, particularly with respect to ligand binding [Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663]. For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. In addition to the teachings herein, these references provide amply guidance for how to generate ActRII variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRII, as demarcated by the outermost of these conserved cysteines, corresponds to positions 30-110 of SEQ ID NO: 1 (ActRII precursor). Therefore, the structurally less-ordered amino acids flanking these cysteine-demarcated core sequences can be truncated by about 1,2,3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1,2,3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRII extracellular domains truncations include SEQ ID NOs: 2 and 3.

Accordingly, a general formula for an active portion (e.g., ligand binding) of ActRII is a polypeptide that comprises, consists essentially of, or consists of amino acids 30-110 of SEQ ID NO: 1. Therefore ActRII polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRII beginning at a residue corresponding to any one of amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113,114, 115, 116, 117, 118, 119, 120, 121, 122, 123, 124, 125, 126, 127, 128, 129, 130, 131, 132, 133, 134, or 135) of SEQ ID NO: 1. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 1, and end at a position selected from 111-135 (e.g., ending at any one of amino acids 111, 112, 113,114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113,114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113,114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113,114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113,114, 115, 116, 117, 118, 119, 120, 121, 122, 123, 124, 125, 126, 127, 128, 129, 130, 131, or 132), or 111-131 (e.g., ending at any one of amino acids 110, 111, 112, 113,114, 115, 116, 117, 118, 119, 120, 121, 122, 123, 124, 125, 126, 127, 128, 129, 130, or 131) of SEQ ID NO: 1. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 1. Thus, in some embodiments, an ActRII polypeptide may comprise, consists essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1. Optionally, ActRII polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1, and comprising no more than 1,2,5, 10 or 15 conservative amino acid changes in the ligand-binding pocket. In some embodiments, the ActRII polypeptide is part of a homodimer protein complex.

In certain embodiments, the disclosure relates to an ActRII polypeptide (e.g., ActRIIA polypeptides, ActRIIB polypeptides, or combinations thereof), which includes fragments, functional variants, and modified forms thereof as well as uses thereof (e.g., treating, preventing, or reducing the pulmonary hypertension associated with lung disease (e.g., pulmonary hypertension associated with COPD, ILD, or CPFE). Preferably, ActRII polypeptides are soluble (e.g., an extracellular domain of ActRII). In some embodiments, ActRII polypeptides inhibit (e.g., Smad signaling) of one or more GDF/BMP ligands [e.g., GDF11, GDF8, activin A, activin B, GDF3, BMP4, BMP6, BMP10, and/or BMP15]. In some embodiments, ActRII polypeptides bind to one or more GDF/BMP ligands [e.g., GDF11, GDF8, activin A, activin B, GDF3, BMP4, BMP6, BMP10, and/or BMP15]. In some embodiments, ActRII polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRII beginning at a residue corresponding to amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113,114, 115, 116, 117, 118, 119, 120, 121, 122, 123, 124, 125, 126, 127, 128, 129, 130, 131, 132, 133, 134 or 135) of SEQ ID NO: 1. In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 1. In certain embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 1. In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1,2,3, 23, 27, 30, and 41.

In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 23. In some alternative embodiments, the ActRII polypeptide (e.g., SEQ ID NO: 23) may lack the C-terminal lysine. In some embodiments, the ActRII polypeptide lacking the C-terminal lysine is SEQ ID NO: 41. In some embodiments, the ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 41. In some embodiments, a patient is administered an ActRII polypeptide comprising, consisting, or consisting essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 23. In some embodiments, a patient is administered an ActRII polypeptide comprising, consisting, or consisting essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 41. In some embodiments, a patient is administered a combination of SEQ ID NO: 23 and SEQ ID NO: 41.

In certain aspects, the present disclosure relates to ActRII polypeptides (e.g., ActRIIA polypeptides, ActRIIB polypeptides, or combinations thereof). In some embodiments, ActRII traps of the present disclosure are variant ActRII polypeptides (e.g., ActRIIA polypeptides, ActRIIB polypeptides, or combinations thereof) that comprise one or more mutations (e.g., amino acid additions, deletions, substitutions, and combinations thereof) in the extracellular domain (also referred to as the ligand-binding domain) of an ActRII polypeptide (e.g., a “wild-type” or unmodified ActRII polypeptide) such that the variant ActRII polypeptide has one or more altered ligand-binding activities than the corresponding wild-type ActRII polypeptide. In some embodiments, variant ActRII polypeptides of the present disclosure retain at least one similar activity as a corresponding wild-type ActRII polypeptide. For example, preferable ActRII polypeptides bind to and inhibit (e.g. antagonize) the function of activin, GDF11 and/or GDF8. In some embodiments, ActRII polypeptides of the present disclosure further bind to and inhibit one or more of ligand of the GDF/BMP [e.g., GDF11, GDF8, activin A, activin B, GDF3, BMP4, BMP6, BMP10, and/or BMP15]. Accordingly, the present disclosure provides ActRII polypeptides that have an altered binding specificity for one or more ActRII ligands.

To illustrate, one or more mutations may be selected that increase the selectivity of the altered ligand-binding domain for GDF11 and/or GDF8 over one or more ActRII-binding ligands such as activins (activin A or activin B), particularly activin A. Optionally, the altered ligand-binding domain has a ratio of K_{d} for activin binding to K_{d} for GDF11 and/or GDF8 binding that is at least 2-, 5-, 10-, 20-, 50-, 100- or even 1000-fold greater relative to the ratio for the wild-type ligand-binding domain. Optionally, the altered ligand-binding domain has a ratio of IC₅₀ for inhibiting activin to IC₅₀ for inhibiting GDF11 and/or GDF8 that is at least 2-, 5-, 10-, 20-, 50-, 100- or even 1000-fold greater relative to the wild-type ligand-binding domain. Optionally, the altered ligand-binding domain inhibits GDF11 and/or GDF8 with an IC₅₀ at least 2-, 5-, 10-, 20-, 50-, 100- or even 1000-times less than the IC₅₀ for inhibiting activin.

In certain embodiments, the present disclosure contemplates specific mutations of an ActRII polypeptide (e.g., ActRIIA polypeptides, ActRIIB polypeptides, or combinations thereof) so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, polypeptides of the present disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of an ActRII polypeptide (e.g., ActRIIA polypeptides, ActRIIB polypeptides, or combinations thereof) as well as truncation mutants. Pools of combinatorial mutants are especially useful for identifying functionally active (e.g., GDF/BMP ligand binding) ActRII sequences. The purpose of screening such combinatorial libraries may be to generate, for example, polypeptides variants, which have altered properties, such as altered pharmacokinetic or altered ligand binding. A variety of screening assays are provided below, and such assays may be used to evaluate variants. For example, ActRII variants may be screened for ability to bind to one or more GDF/BMP ligands [e.g., GDF11, GDF8, activin A, activin B, GDF3, BMP4, BMP6, BMP10, and/or BMP15], to prevent binding of a GDF/BMP ligand to an ActRII polypeptide, as well as heteromultimers thereof, and/or to interfere with signaling caused by an GDF/BMP ligand.

The activity of ActRII polypeptides (e.g., ActRIIA polypeptides, ActRIIB polypeptides, or combinations thereof) or variants thereof may also be tested in a cell-based or in vivo assay. For example, the effect of an ActRII polypeptide on the expression of genes involved in pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) pathogenesis may be assessed. This may, as needed, be performed in the presence of one or more recombinant ligand proteins [e.g., GDF11, GDF8, activin A, activin B, GDF3, BMP4, BMP6, BMP10, and/or BMP15], and cells may be transfected so as to produce an ActRII polypeptide, and optionally, an GDF/BMP ligand. Likewise, an ActRII polypeptide may be administered to a mouse or other animal and effects on pulmonary hypertension associated with lung disease (e.g., pulmonary hypertension associated with COPD, ILD, or CPFE) pathogenesis may be assessed using art-recognized methods. Similarly, the activity of an ActRII polypeptide or variant thereof may be tested in blood cell precursor cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference ActRII polypeptide (e.g., ActRIIA polypeptides, ActRIIB polypeptides, or combinations thereof). Such variants, when expressed from recombinant DNA constructs, can be used in gene therapy protocols. Likewise, mutagenesis can give rise to variants which have intracellular half-lives dramatically different than the corresponding unmodified ActRII polypeptide. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter the half-life of the ActRII polypeptide.

A combinatorial library may be produced by way of a degenerate library of genes encoding a library of polypeptides which each include at least a portion of potential ActRII polypeptide sequences. For instance, a mixture of synthetic oligonucleotides can be enzymatically ligated into gene sequences such that the degenerate set of potential ActRII encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art [Narang, SA (1983) Tetrahedron 39:3; Itakura et al. (1981) Recombinant DNA, Proc. 3rd Cleveland Sympos. Macromolecules, ed. AG Walton, Amsterdam: Elsevier pp 273-289; Itakura et al. (1984) Annu. Rev. Biochem. 53:323; Itakura et al. (1984) Science 198:1056; and Ike et al. (1983) Nucleic Acid Res. 11:477]. Such techniques have been employed in the directed evolution of other proteins [Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos. 5,223,409, 5,198,346, and 5,096,815].

Alternatively, other forms of mutagenesis can be utilized to generate a combinatorial library. For example, ActRII polypeptides of the disclosure (e.g., ActRIIA polypeptides, ActRIIB polypeptides, or combinations thereof) can be generated and isolated from a library by screening using, for example, alanine scanning mutagenesis [Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [Gustin et al. (1993) Virology 193:653-660; and Brown et al. (1992) Mol. Cell Biol. 12:2644-2652; McKnight et al. (1982) Science 232:316], by saturation mutagenesis [Meyers et al., (1986) Science 232:613]; by PCR mutagenesis [Leung et al. (1989) Method Cell Mol Biol 1:11-19]; or by random mutagenesis, including chemical mutagenesis [Miller et al. (1992) A Short Course in Bacterial Genetics, CSHL Press, Cold Spring Harbor, NY; and Greener et al. (1994) Strategies in Mol Biol 7:32-34]. Linker scanning mutagenesis, particularly in a combinatorial setting, is an attractive method for identifying truncated (bioactive) forms of ActRII polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of ActRII polypeptides (e.g., ActRIIA polypeptides, ActRIIB polypeptides, or combinations thereof). The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Exemplary assays include ligand [e.g., GDF11, GDF8, activin A, activin B, GDF3, BMP4, BMP6, BMP10, and/or BMP15]binding assays and/or ligand-mediated cell signaling assays.

As will be recognized by one of skill in the art, most of the described mutations, variants or modifications described herein may be made at the nucleic acid level or, in some cases, by post-translational modification or chemical synthesis. Such techniques are well known in the art and some of which are described herein. In part, the present disclosure identifies functionally active portions (fragments) and variants of ActRII polypeptides (e.g., ActRIIA polypeptides, ActRIIB polypeptides, or combinations thereof) that can be used as guidance for generating and using other variant ActRII polypeptides within the scope of the disclosure provided herein.

In certain embodiments, functionally active fragments of ActRII polypeptides of the present disclosure can be obtained by screening polypeptides recombinantly produced from the corresponding fragment of the nucleic acid encoding an ActRII polypeptide. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (inhibitors) of ActRII receptors and/or one or more ligands [e.g., GDF11, GDF8, activin A, activin B, GDF3, BMP4, BMP6, BMP10, and/or BMP15].

In certain embodiments, ActRII polypeptides of the present disclosure (e.g., ActRIIA polypeptides, ActRIIB polypeptides, or combinations thereof) may further comprise post-translational modifications in addition to any that are naturally present in the ActRII polypeptide. Such modifications include, but are not limited to, acetylation, carboxylation, glycosylation, phosphorylation, lipidation, and acylation. As a result, the ActRII polypeptide may contain non-amino acid elements, such as polyethylene glycols, lipids, polysaccharide or monosaccharide, and phosphates. Effects of such non-amino acid elements on the functionality of a ligand trap polypeptide may be tested as described herein for other ActRII variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, W138, NIH-3T3 or HEK293) have specific cellular machinery and characteristic mechanisms for such post-translational activities and may be chosen to ensure the correct modification and processing of the ActRII polypeptides.

In certain aspects, ActRII polypeptides of the present disclosure (e.g., ActRIIA polypeptides, ActRIIB polypeptides, or combinations thereof) include fusion proteins having at least a portion (domain) of an ActRII polypeptide and one or more heterologous portions (domains). Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the ActRII polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation. Other types of fusion domains that may be selected include multimerizing (e.g., dimerizing, tetramerizing) domains and functional domains (that confer an additional biological function) including, for example constant domains from immunoglobulins (e.g., Fc domains).

In certain aspects, ActRII polypeptides of the present disclosure (e.g., ActRIIA polypeptides, ActRIIB polypeptides, or combinations thereof) contain one or more modifications that are capable of “stabilizing” the polypeptides. By “stabilizing” is meant anything that increases the in vitro half-life, serum half-life, regardless of whether this is because of decreased destruction, decreased clearance by the kidney, or other pharmacokinetic effect of the agent. For example, such modifications enhance the shelf-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising an ActRII polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes nonproteinaceous modifications such as a carbohydrate moiety, or nonproteinaceous moiety, such as polyethylene glycol. In certain embodiments, an ActRII polypeptide is fused with a heterologous domain that stabilizes the polypeptide (a “stabilizer” domain), preferably a heterologous domain that increases stability of the polypeptide in vivo. Fusions with a constant domain of an immunoglobulin (e.g., a Fc domain) are known to confer desirable pharmacokinetic properties on a wide range of proteins. Likewise, fusions to human serum albumin can confer desirable properties.

An example of a native amino acid sequence that may be used for the Fc portion of human IgG1 (G1Fc) is shown below (SEQ ID NO: 11). Dotted underline indicates the hinge region, and solid underline indicates positions with naturally occurring variants. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 11. Naturally occurring variants in G1Fc would include E134D and M136L according to the numbering system used in SEQ ID NO: 11 (see Uniprot P01857).

| | (SEQ ID NO: 11) |
|---|---|
| 1 | 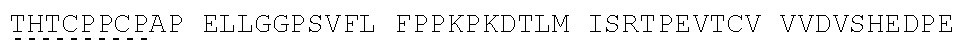 |
| 51 | VKFNWYVDGV EVHNAKTKPR EEQYNSTYRV VSVLTVLHQD WINGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

Optionally, the IgG1 Fc domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcγ receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain.

An example of a native amino acid sequence that may be used for the Fc portion of human IgG2 (G2Fc) is shown below (SEQ ID NO: 12). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 12.

| | (SEQ ID NO: 12) |
|---|---|
| 1 | 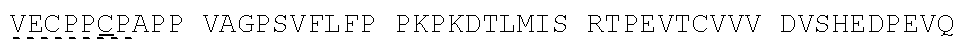 |
| 51 | FNWYVDGVEV HNAKTKPREE QFNSTERVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVES |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 13) contains a short hinge region consisting of a single 15-residue segment, whereas the second G3Fc sequence (SEQ ID NO: 14) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80% b, 85%, 86%, 87%, 88%, 89%, 90%, 91%, 92%, 93%, 94%, 95%, 96%, 97%, 98%, 99%, or 100% identity to SEQ ID NOs: 13 and 14.

(SEQ ID NO: 13)

| | |
|---|---|
| 1 | 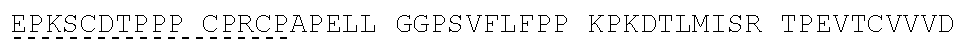 |
| 51 | VSHEDPEVQF KWYVDGVEVH NAKTKPREEQ YNSTERVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP GK |
| | (SEQ ID NO: 14) |
| 1 | 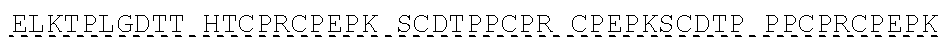 |
| 51 | 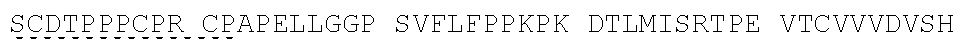 |
| 101 | EDPEVOFKWY VDGVEVHNAK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 13, and the present disclosure provides fusion proteins comprising G3Fc domains containing one or more of these variations. In addition, the human immunoglobulin IgG3 gene (IGHG3) shows a structural polymorphism characterized by different hinge lengths (see Uniprot P01860). Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 15). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 15.

| | (SEQ ID NO: 15) |
|---|---|
| 1 | 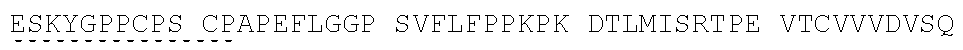 |
| 51 | EDPEVQFNWY VDGVEVHNAK TKPREEQENS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVESCSVM HEALHNHYTQ KSLSLSLGK |

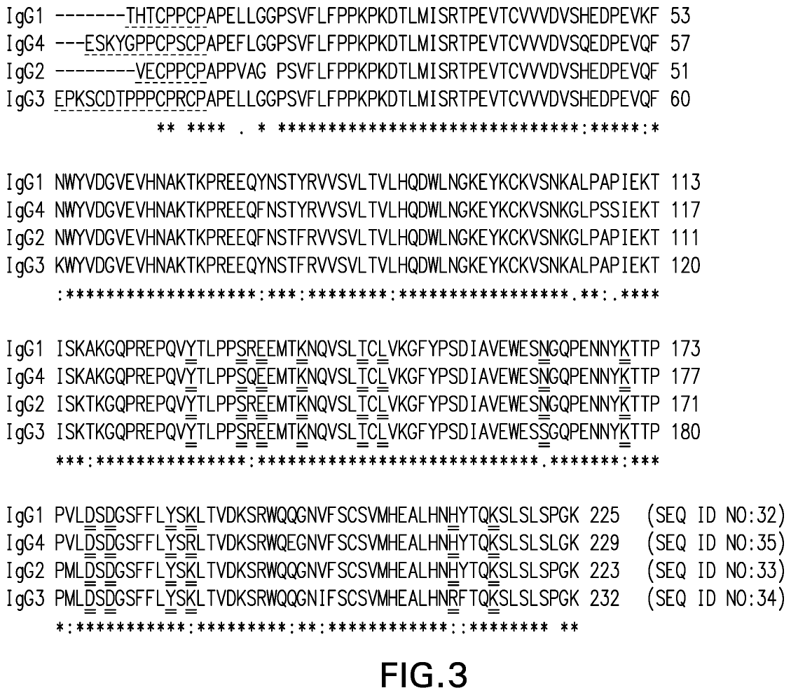

A variety of engineered mutations in the Fc domain are presented herein with respect to the G1Fc sequence (SEQ ID NO: 11), and analogous mutations in G2Fc, G3Fc, and G4Fc can be derived from their alignment with G1Fc in FIG. 3. Due to unequal hinge lengths, analogous Fc positions based on isotype alignment (FIG. 3) possess different amino acid numbers in SEQ ID NOs: 11, 12, 13, 14, and 15. It can also be appreciated that a given amino acid position in an immunoglobulin sequence consisting of hinge, C_{H}2, and C_{H}3 regions (e.g., SEQ ID NOs: 11, 12, 13, 14, and 15) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 11), the human IgG1 heavy chain constant domain (Uniprot P01857), and the human IgG1 heavy chain is as follows.

Correspondence of C_{H}3 Positions in Different Numbering Systems

| | IgG1 | IgG1 |
|---|---|---|
| G1Fc | heavy chain | heavy chain |
| (Numbering begins | constant domain | (EU numbering |
| at first threonine | (Numbering | scheme of Kabat |
| in hinge region) | begins at C_{H}1) | et al., 1991*) |
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

*Kabat et al. (eds) 1991; pp. 688-696 in Sequences of Proteins of Immunological Interest, 5^{th} ed., Vol. 1, NIH, Bethesda, MD.

Various methods are known in the art that increase desired pairing of Fc-containing fusion polypeptide chains in a single cell line to produce an asymmetric fusion protein at acceptable yields [Klein et al (2012) mAbs 4:653-663; and Spiess et al (2015) Molecular Immunology 67(2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing [Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605].

It is understood that different elements of the fusion proteins (e.g., immunoglobulin Fc fusion proteins) may be arranged in any manner that is consistent with desired functionality. For example, an ActRII polypeptide domain may be placed C-terminal to a heterologous domain, or alternatively, a heterologous domain may be placed C-terminal to an ActRII polypeptide domain. The ActRII polypeptide domain and the heterologous domain need not be adjacent in a fusion protein, and additional domains or amino acid sequences may be included C- or N-terminal to either domain or between the domains.

For example, an ActRII receptor fusion protein may comprise an amino acid sequence as set forth in the formula A-B-C. The B portion corresponds to an ActRII polypeptide domain (e.g., ActRIIA polypeptides, ActRIIB polypeptides, or combinations thereof). The A and C portions may be independently zero, one, or more than one amino acid, and both the A and C portions when present are heterologous to B. The A and/or C portions may be attached to the B portion via a linker sequence. A linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 16), GGGG (SEQ ID NO: 17), TGGGG (SEQ ID NO: 18), SGGGG (SEQ ID NO: 19), TGGG (SEQ ID NO: 20), SGGG (SEQ ID NO: 21), or GGGGS (SEQ ID NO: 22) singlets, or repeats. In certain embodiments, an ActRII fusion protein comprises an amino acid sequence as set forth in the formula A-B-C, wherein A is a leader (signal) sequence, B consists of an ActRII polypeptide domain, and C is a polypeptide portion that enhances one or more of in vivo stability, in vivo half-life, uptake/administration, tissue localization or distribution, formation of protein complexes, and/or purification. In certain embodiments, an ActRII fusion protein comprises an amino acid sequence as set forth in the formula A-B-C, wherein A is a TPA leader sequence, B consists of an ActRII receptor polypeptide domain, and C is an immunoglobulin Fc domain. Exemplary fusion proteins comprise the amino acid sequence set forth in any one of SEQ ID NOs: 23, 27, 30, and 41.

In some embodiments, ActRII polypeptides to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of purity are well known in the art [see, e.g., Flatman et al., (2007) J. Chromatogr. B 848:79-87]. In some embodiments, ActRII polypeptides to be used in accordance with the methods described herein are recombinant polypeptides.

ActRII polypeptides of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the polypeptides of the disclosure, including fragments or variants thereof, may be recombinantly produced using various expression systems [e.g., E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length ActRII polypeptides by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites. Alternatively, such polypeptides may be produced from recombinantly generated full-length ActRII polypeptides using chemical cleavage (e.g., cyanogen bromide, hydroxylamine, etc.).

### 3. Nucleic Acids Encoding ActRII Polypeptides

In certain embodiments, the present disclosure provides isolated and/or recombinant nucleic acids encoding ActRII polypeptides (e.g., ActRIIA polypeptides, ActRIIB polypeptides, or combinations thereof) including fragments, functional variants, and fusion proteins thereof.

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding ActRII polypeptides of the disclosure are understood to include nucleic acids that are variants of any one of SEQ ID NOs: 4, 5, or 28. Variant nucleotide sequences include sequences that differ by one or more nucleotide substitutions, additions, or deletions including allelic variants, and therefore, will include coding sequence that differ from the nucleotide sequence designated in any one of SEQ ID NOs: 4, 5, or 28.

In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to any one of SEQ ID NOs: 4, 5, or 28. One of ordinary skill in the art will appreciate that nucleic acid sequences that are at least 70%, 75%, 80%, 85%, 90%, 91%, 92%, 93%, 94% 95%, 96%, 97%, 98%, 99%, or 100% identical to the sequences complementary to SEQ ID NOs: 4, 5, or 28, and variants thereof, are also within the scope of the present disclosure. In further embodiments, the nucleic acid sequences of the disclosure can be isolated, recombinant, and/or fused with a heterologous nucleotide sequence, or in a DNA library.

In other embodiments, nucleic acids of the present disclosure also include nucleotide sequences that hybridize under highly stringent conditions to the nucleotide sequence designated in SEQ ID NOs: 4, 5, or 28, complement sequences of SEQ ID NOs: 4, 5, or 28, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 4, 5, or 28 to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art and can be used in a variety of host cells. Typically, one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and can vary with the host cell used.

In certain aspects, the subject nucleic acid disclosed herein is provided in an expression vector comprising a nucleotide sequence encoding an ActRII polypeptide (e.g., ActRIIA polypeptides, ActRIIB polypeptides, or combinations thereof) operably linked to at least one regulatory sequence. Regulatory sequences are art-recognized and are selected to direct expression of the ActRII polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding an ActRII polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ActRII polypeptide include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNA1/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUWi), and pBlueBac-derived vectors (such as the ß-gal containing pBlueBac III).

In one embodiment, a vector will be designed for production of the subject ActRII polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the subject ActRII polypeptides in cells propagated in culture, e.g., to produce proteins, including fusion proteins or variant proteins, for purification.

This disclosure also pertains to a host cell transfected with a recombinant gene including a coding sequence for one or more of the subject ActRII polypeptides. The host cell may be any prokaryotic or eukaryotic cell. For example, an ActRII polypeptide of the disclosure may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject ActRII polypeptides. For example, a host cell transfected with an expression vector encoding an ActRII polypeptide can be cultured under appropriate conditions to allow expression of the ActRII polypeptide to occur. The polypeptide may be secreted and isolated from a mixture of cells and medium containing the polypeptide. Alternatively, the ActRII polypeptide may be retained cytoplasmically or in a membrane fraction and the cells harvested, lysed and the protein isolated. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffimity purification with antibodies specific for particular epitopes of the ActRII polypeptides, and affinity purification with an agent that binds to a domain fused to the ActRII polypeptide (e.g., a protein A column may be used to purify an ActRII-Fc fusion proteins). In some embodiments, the ActRII polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. An ActRII protein may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant ActRII polypeptide, can allow purification of the expressed fusion protein by affinity chromatography using a Ni²⁺ metal resin. The purification leader sequence can then be subsequently removed by treatment with enterokinase to provide the purified ActRII polypeptide. See, e.g., Hochuli et al. (1987) J. Chromatography 411:177; and Janknecht et al. (1991) PNAS USA 88:8972.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et 5 al., John Wiley & Sons: 1992.

### 4. Methods of Use

In part, the present disclosure relates to methods of treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) comprising administering to a patient in need thereof an effective amount of an ActRII polypeptide as described herein. In some embodiments, the disclosure contemplates methods of treating, preventing, or reducing the progression rate and/or severity of one or more complications of pulmonary hypertension associate with lung disease (e.g., pulmonary hypertension associated with chronic obstructive pulmonary disease (COPD), interstitial lung disease (ILD), or combined pulmonary fibrosis and emphysema (CPFE)), comprising administering to a patient in need thereof an effective amount of an ActRII polypeptide as described herein. In some embodiments, the ActRII polypeptide is administered at a dosing range of 0.1 mg/kg to 2.0 mg/kg (e.g., 0.3 mg/kg or 0.7 mg/kg). In some embodiments, the administration of an ActRII polypeptide results in a change of one or more hemodynamic or functional parameters (e.g., a reduction in pulmonary vascular resistance (PVR); an increase in 6-minute walk distance (6MWD); a decrease of the N-terminal pro B-type natriuretic peptide (NT-proBNP) levels; a prevention or delay in pulmonary hypertension Functional Class progression as recognized by the World Health Organization (WHO); a promotion or increase in pulmonary hypertension Functional Class regression as recognized by the WHO; an improvement in right ventricular function; and an improvement in pulmonary artery pressure).

In certain aspects, the disclosure relates to methods of treating pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the method reduces the right ventricular systolic pressure (RVSP) by at least 10%.

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1. In some embodiments, the one or more complications of pulmonary hypertension associated with lung disease is selected from the group consisting of persistent cough, productive cough, wheezing, exercise intolerance, respiratory infections, bronchiectasis, chronic infections, nasal polyps, hemoptysis, pneumothorax, respiratory failure, dyspnea, chest pain, hemoptysis, pneumothorax, pulmonary vascular remodeling, pulmonary fibrosis, pulmonary vascular endothelial dysfunction, hypoxia due to chronic pulmonary injury, hypoxic pulmonary vasoconstriction, inflammation, smooth muscle hypertrophy, and right ventricular hypertrophy.

These methods are particularly aimed at therapeutic and prophylactic treatments of animals, and more particularly, humans. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The terms “treatment”, “treating”, “alleviating”, “reducing the progression rate”, “reducing the severity of”, and the like are used herein to generally mean obtaining a desired pharmacologic and/or physiologic effect, and may also be used to refer to improving, alleviating, and/or decreasing the severity of one or more clinical complication of a condition being treated (e.g., pulmonary hypertension associated with lung disease). The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure (e.g., pulmonary hypertension associated with lung disease) is achieved by administering one or more ActRII polypeptides of the present disclosure in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

In certain aspects, the disclosure contemplates the use of an ActRII polypeptide, in combination with one or more additional active agents or other supportive therapy for treating or preventing a disease or condition (e.g., pulmonary hypertension associated with lung disease). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more ActRII polypeptides of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the ActRII polypeptide of the present disclosure with the additional active agent or therapy and/or the desired effect.

### WHO Classiflcation Outine

Pulmonary hypertension conditions treated by methods describe herein, can comprise any one or more of the conditions recognized according to the World Health Organization (WHO). See, e.g., Simonneau (2019) Eur RespiriJ: 53:1801913.

TABLE 1
Clinical Classification of Pulmonary Hypertension

```
Group 1: Pulmonary arterial hypertension (PAH)
  1.1 Idiopathic PAH
  1.2 Heritable PAH
    1.2.1 BMPR2
    1.2.2 ALK-1, ENG, SMAD9, CAV1, KCNK3
    1.2.3 Unknown
  1.3 Drug and toxin induced PAH
  1.4 Associated with:
    1.4.1 Connective tissue disease
    1.4.2 HIV infection
    1.4.3 Portal hypertension
    1.4.4 Congenital heart diseases
    1.4.5 Schistosomiasis
  1.5 PAH long-term responders to calcium channel blockers
  1.6 PAH with overt features of venous/capillaries (PVOD/PCH) involvement
  1.7 Persistent PH of the newborn syndrome
Group 2: Pulmonary hypertension due to left heart disease
  2.1 PH due to heart failure with preserved LVEF¹ (HFpEF)
  2.2 PH due to heart failure with reduced LVEF (HFrEF)
  2.3 Valvular heart disease
  2.4 Congenital/acquired cardiovascular conditions leading to post-capillary PH
Group 3: Pulmonary hypertension due to lung disease and/or hypoxia
  3.1 Obstructive lung disease
  3.2 Restrictive lung disease
  3.3 Other lung disease with mixed restrictive/obstructive pattern
  3.4 Hypoxia without lung disease
  3.5 Developmental lung disorders
Group 4: Pulmonary hypertension due to pulmonary artery obstructions
  4.1 Chronic thromboembolic PH
  4.2 Other pulmonary artery obstructions
    4.2.1 Sarcoma (high or intermediate grade) or angiosarcoma
    4.2.2 Other malignant tumours
      Renal carcinoma
      Uterine carcinoma
      Germ cell tumours of the testis
      Other tumours
    4.2.3 Non-malignant tumours
      Uterine leiomyoma
    4.2.4 Arteritis without connective tissue disease
    4.2.5 Congenital pulmonary artery stenoses
    4.2.6 Parasites
      Hydatidosis
Group 5: Pulmonary hypertension with unclear and/or multifactorial mechanisms.
  5.1 Hematological disorders (e.g., Chronic hemolytic anaemia and myeloproliferative
  disorders)
  5.2 Systemic and metabolic disorders (e.g., Pulmonary Langerhans cell histiocytosis,
  Gaucher disease, Glycogen storage disease, Neurofibromatosis, and Sarcoidosis)
  5.3 Others (e.g., Chronic renal failure with or without haemodialysis and Fibrosing
  mediastinitis)
  5.4 Complex congenital heart disease
¹Left ventricular ejection fraction
```

As used herein, the term “pulmonary hemodynamic parameter” refers to any parameter used to describe or evaluate the blood flow through the heart and pulmonary vasculature. Examples of pulmonary hemodynamic parameters include, but are not limited to, mean pulmonary artery pressure (mPAP), diastolic pulmonary artery pressure (dPAP) [also known as pulmonary artery diastolic pressure (PADP)], systolic pulmonary artery pressure (sPAP) [also known as pulmonary artery systolic pressure (PASP)], mean right atrial pressure (mRAP), pulmonary capillary wedge pressure (PCWP) [also known as pulmonary artery wedge pressure (PAWP)], pulmonary vascular resistance (PVR) and cardiac output (CO).

Many of the pulmonary hemodynamic parameters described above are interrelated. For example, PVR is related to mPAP, PCWP and CO according to the following equation:

PVR=(mPAP−PCWP)/CO [Woods Units]

The PVR measures the resistance to flow imposed by the pulmonary vasculature without the influence of the left-sided filling pressure. PVR can also be measured according to the following equations:

PVR=TPG×80/CO [unit: dynes-sec-cm⁻⁵] OR PVR=(mPAP −PCWP)×80/CO [unit: dynes-sec-cm⁻⁵]

In some embodiments, the total PVR can be measured using the following equation:

TPR=mPAP/CO.

In some embodiments, the normal PVR is 20-180 dynes-sec-cm-5 or typically less than 0.5-2 Wood units. According to some embodiments, an elevated PVR may refer to a PVR above 2 Wood units, above 2.5 Wood units, above 3 Wood units or above 3.5 Wood units.

As yet another example, mPAP is related to dPAP and sPAP according to the following equation: mPAP=(⅔) dPAP+(⅓) sPAP.

In some embodiments, the pulmonary hemodynamic parameters are measured directly, such as during a right heart catheterization. In other embodiments, the pulmonary hemodynamic parameters are estimated and/or evaluated through other techniques such as magnetic resonance imaging (MRI) or echocardiography.

Exemplary pulmonary hemodynamic parameters include mPAP, PAWP, and PVR. The one or more pulmonary hemodynamic parameters may be measured by any appropriate procedures, such as by utilizing a right heart catheterization or echocardiography. Various hemodynamic characteristics of PH and pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) are shown in Table 2.

TABLE 2
Hemodynamic Characteristics of Pulmonary Hypertension (PH)
and Pulmonary Hypertension Associated with Lung Disease

| | Hemodynamic Characteristics |
|---|---|
| Pulmonary | mPAP >20 mmHg |
| Hypertension | |
| Pulmonary | mPAP >20 mmHg |
| hypertension | PAWP ≤15 mmHg |
| associated with | PVR ≥3 Wood units |
| lung disease | |

The clinical classification or hemodynamic characteristics of PAH described herein and the associated diagnostic parameters may be updated or varied based on the availability of new or existing sources of data or when additional clinical entities are considered.

Characteristics of Pulmonary Hypertension Associated with Lung Disease

Pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) (WHO Group 3 PH) is the second most common form of pulmonary hypertension and is associated with increased morbidity and mortality. Patients with Group 3 pulmonary hypertension have a worse outcome than patients with Group 1 pulmonary hypertension. Similarly, patients with Group 1 pulmonary arterial hypertension and associated lung disease suffer from even worse outcomes relative to patients having only Group 1 pulmonary arterial hypertension.

A variety of factors contribute to the pathogenesis of pulmonary hypertension associated with lung disease. These factors vary based on the underlying lung disease. For example, in pulmonary hypertension caused by COPD, the most prominent etiology for pulmonary hypertension is hypoxic pulmonary vasoconstriction (HPVC) with remodeling of the pulmonary vascular bed. Initial changes during vascular remodeling include distal neomuscularization of the arterioles, intimal thickening, and medial hypertrophy. This remodeling eventually leads to fewer blood vessels and consequently increased peripheral vascular resistance seen in pulmonary hypertension. Additional mechanisms underlying ILD-associated pulmonary hypertension include vascular destruction due to progressive parenchymal fibrosis, vascular inflammation, perivascular fibrosis, thrombotic angiopathy, and endothelial dysfunction. More specifically, patients with pulmonary hypertension associated with idiopathic pulmonary fibrosis (IPF) may have an abnormal vascular phenotype, characterized by aberrant gene expression profiles that promote vascular remodeling.

Pulmonary hypertension associated with lung disease can be diagnosed on a mean pulmonary artery pressure (mPAP) of or above 25 mmHg. Pulmonary hypertension associated with lung disease can lead to persistent cough, productive cough, wheezing, exercise intolerance, respiratory infections, bronchiectasis, chronic infections, nasal polyps, hemoptysis, pneumothorax, respiratory failure, dyspnea, chest pain, hemoptysis, pneumothorax, pulmonary vascular remodeling, pulmonary fibrosis, pulmonary vascular endothelial dysfunction, hypoxia due to chronic pulmonary injury, hypoxic pulmonary vasoconstriction, inflammation, smooth muscle hypertrophy, and right ventricular hypertrophy. The lung diseases associated with pulmonary hypertension may be classified as either obstructive lung disease or restrictive lung disease. Obstructive lung diseases (e.g., COPD, cystic fibrosis, asthma, emphysema, and chronic bronchitis) is marked by the difficulty in exhalation. Alternatively, restrictive lung diseases, which can be further separated into intrinsic (e.g., pulmonary fibrosis, interstitial lung disease, sarcoidosis, idiopathic pulmonary fibrosis) and extrinsic (obesity, scoliosis, Myasthenia gravis, and pleural effusion) disorders, is characterized by the restriction of full lung expansion.

Pulmonary hypertension associated with lung disease (e.g., pulmonary hypertension associated with lung disease) can be challenging to diagnose due to the heterogeneity of the underlying lung condition. Many symptoms of the lung disease mimic the symptoms of pulmonary hypertension. There are, however, several clinical features that prompt a diagnosis of pulmonary hypertension associated with lung disease (e.g., exertional dyspnea or hypoxemia not fully explained by parenchymal lung disease or a sleep disorder, rapid decline of arterial oxygenation upon exercise, any clinical features suggestive of right-sided heart failure, enlarged pulmonary arteries, attenuation of peripheral pulmonary vasculature, or right ventricular enlargement as demonstrated by high resolution computed tomography (HRCT), severe reductions in diffusing capacity as demonstrated by pulmonary function testing, and lung biopsy). Klings, E. S. (2021). Pulmonary hypertension due to lung disease and/or hypoxemia (group 3 pulmonary hypertension): Epidemiology, pathogenesis, and diagnostic evaluation in adults. UpToDate. Retrieved Apr. 6, 2021 from https://www.uptodate.com/contents/pulmonary-hypertension-due-to-lung-disease-and-or-hypoxemia-group-3-pulmonary-hypertension-epidemiology-pathogenesis-and-diagnostic-evaluation-in-adults.

While echocardiography is a standard test when investigating patients with suspected pulmonary hypertension of unknown etiology for underlying lung disease and/or sleep disordered breathing, echocardiography may not be as reliable for accurately diagnosing pulmonary hypertension in a patient having severe lung disease. In such cases, right heart catheterization (RHC) may give a more accurate assessment.

### Chronic Obstructive Lung Disease

In some embodiments, the disclosure relates to methods of treating pulmonary hypertension associated with chronic obstructive pulmonary disease (COPD), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1. Chronic obstructive lung disease (also, chronic obstructive pulmonary disease (COPD)), is an inflammatory lung disease that causes obstructed airflow from the lungs. Within this group of diseases are emphysema and chronic bronchitis. According to the Centers for Disease Control and Prevention, millions of people suffer with COPD, 16 million of which are in the United States of America.

The severity of COPD is determined using the Global Initiative for Chronic Obstructive Lung Disease (GOLD) staging or grading system, determined by spirometry results (GOLD 1: mild, GOLD 2: moderate, GOLD 3: severe, and GOLD 4: very severe). This system bases the stage of COPD on several factors (e.g., overall symptoms, number of times COPD has worsened, hospitalizations due to worsening COPD, and results from spirometry). The majority of patients with pulmonary hypertension caused by COPD present with severe or very severe airflow obstruction (GOLD spirometric stages 3 or 4, FEV-1<50% of predicted) or severe emphysema, and mild-to-moderate precapillary pulmonary hypertension. Current treatments for COPD include short-acting bronchodilators, long-acting bronchodilators, inhaled steroids, combination inhalers that include both bronchodilators and inhaled steroids or more than one type of bronchodilator, oral steroids, phosphodiesterase-4 inhibitors, theophylline, antibiotics, various types of lung therapies, and in-home non-invasive ventilation therapy.

### Interstitial Lung Diseases

In some embodiments, the disclosure relates to methods of treating pulmonary hypertension associated with interstitial lung disease (ILD), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1. Interstitial lung disease, or ILD, is a chronic lung disease that occurs as a result of damage between the air sacs in the lungs that leads to lung scarring, inflammation, and breathing problems. ILDs may be caused by infections, medicines, and inhalation of harmful particles in the air. The underlying cause of the ILD determines the course of treatment. ILDs overall decrease the quality of life of the person living with the disease, and may shorten the person's life altogether.

There are approximately five categories of ILDs based on their underlying causes: ILDs caused by exposure or occupational related (e.g., asbestosis, silicosis, hypersensitivity pneumonitis), ILDs related to medications and/or medical treatments (e.g., chemotherapy, radiation therapy), ILDs associated with autoimmune disorders and/or connective tissue diseases (e.g., lupus, scleroderma, poly or dermatomyositis, rheumatoid arthritis), sarcoidosis, and idiopathic ILDs. Outside of the five general categories remains ILDs such as idiopathic pulmonary fibrosis (IPF), bronchiolitis obliterans, histiocytosis X, chronic eosinophilic pneumonia, collagen vascular disease, granulomatous vasculitis, Goodpasture's syndrome, and pulmonary alveolar proteinosis.

The symptoms of ILDs may vary from person to person, as well as based on the particular ILD, however the common link between the various forms of ILD is that all ILDs begin with an inflammation in the bronchioles (e.g., bronchiolitis), alveoli (e.g., alveolitis), or capillaries (vasculitis). The most common symptoms of ILDs, such as shortness of breath (especially with exertion), fatigue and weakness, loss of appetite, loss of weight, dry cough that does not produce phlegm, discomfort in the chest, labored breathing, and hemorrhage in the lungs, may resemble other lung conditions or medical problems.

Fibrosis results in permanent destruction of air sacs, the lung tissue between and surrounding the airs sacs, and the lung capillaries. Disease progression may be gradual or rapid and present with very mild, moderate, or very severe symptoms. The course of ILDs is unpredictable, but may improve with medical intervention.

Interstitial lung diseases are diagnosed using pulmonary function tests (PFTs), chest X-rays, blood tests (e.g., analysis of arterial blood gas to determine the amount of carbon dioxide and oxygen is in the blood), high-resolution computed tomography (HRCT, CT, or CAT scan), bronchoscopy, bronchoalveolar lavage, and lung biopsy.

Treatment plans for ILDs are typically determined based on a person's age, overall health, and medical history, extent of the disease, a person's tolerance for specific medications, procedures, and/or therapies, expectations for the course of the disease, as well as a person's opinion or preference. These treatment plans may include oral medications (e.g., corticosteroids), oxygen supplementation, and lung transplantation.

### Idiopathic Pulmonaryfibrosis and Other Idiopathic Interstitial Pneumonias

In some embodiments, the disclosure relates to methods of treating pulmonary hypertension associated with idiopathic pulmonary fibrosis (IPF), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1. Idiopathic pulmonary fibrosis (IPF) (also, cryptogenic fibrosing alveolitis, chronic idiopathic fibrosing alveolitis, interstitial pneumonia) is one of the most frequently diagnosed interstitial lung diseases (ILDs), affecting approximately 13 to 20 per 100,000 people worldwide, with 30,000 to 40,000 new cases diagnosed each year. Though there are medical treatments for IPF available, the disease remains severe with an expectation of clinical decline.

There are several underlying factors that influence the progression of IPF, one of which is thought to be chronic and/or repetitive microinjuries of the alveolar epithelium (e.g., exposure to environmental pollutants, acid aspiration due to gastroesophageal reflux, and viral infections). Damage to the epithelium is followed by injury and/or activation of cells lining the vascular and interstitial compartments of the lung, epithelium of distal airways, ad resident macrophages. Genetic factors may contribute to IPF, which is suggested by the occurrence of IPF-like disease in patients with rare genetic disorders and by cases of familial idiopathic interstitial pneumonia.

There are currently no treatments that have proven effective at stopping the progression of the disease, but newer medications (e.g., pirfenidone and nintedanib) have been approved by the Food and Drug Administration to help slow the progression of the disease.

### Non-Idiopathic Pulmonary Fibrosis Interstitial Lung Disease

In some embodiments, the disclosure relates to methods of treating pulmonary hypertension associated with non-idiopathic pulmonary fibrosis interstitial lung disease (non-IPF ILD), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1. Non-idiopathic pulmonary fibrosis interstitial lung disease (non-IPF) causes inflammation and fibrosis of the lung interstitium leading to impaired gas exchange. The estimated prevalence of non-IPF is estimated to range from 25 to 74 per 100,000 people. There are over 200 known causes for non-IPF that can typically be categorized as occupational and environmental exposures, organic substances causing hypersensitivity pneumonitis, drug-induced lung toxicity, connective tissue diseases, and systemic illnesses.

The pathogenesis of non-IPF is similar between non-IPFs resulting from any one of the over 200 known causes, involving phases of injury (e.g., recurrent and direct epithelial/endothelial injury to distal air spaces, and destruction of the alveolar-capillary basement membrane), inflammation caused by release of proinflammatory cytokines and chemokines by macrophages (e.g., transforming growth factor-beta), and repair (e.g., myofibroblast formation and secretion of fibrous proteins and ground substance that forms the extracellular matrix). This process, however, repeated over time results in continued thickening and irreversible fibrosis of the lung parenchyma.

Treatment and management of the disease involves supportive care, supplemental oxygen, and in certain conditions, corticosteroids. A lung transplant may be considered as an option in severe or progressive cases. Mortality rates can be as high as 100% during an acute exacerbation of non-IPF.

### Combined Pulmonary Fibrosis and Emphysema

In some embodiments, the disclosure relates to methods of treating pulmonary hypertension associated with combined pulmonary fibrosis and emphysema (CPFE), comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1. Combined pulmonary fibrosis and emphysema (CPFE) is characterized by dyspnea, upper-lobe emphysema, lower-lobe fibrosis, and abnormalities of gas exchange. CPFE can be further complicated by pulmonary hypertension, acute lung injury, and lung cancer. Different pulmonary function tests (PFTs) are used to diagnose CPFE than the PFTs used to diagnose fibrosis or emphysema alone. Additionally, HRCT scanning can be used to detect the simultaneous occurrence of emphysema and pulmonary fibrosis.

CPFE has been linked to cigarette smoking, exposure to asbestos and mineral dust, hypersensitivity pneumonitis (or famer lung), as well as being male, and has significant mortality. Median survival has ranged from 2.1 to 8.5 years, and if pulmonary hypertension is present, the 1-year survival is only 60%. Despite this disparity, there is no specific treatment for CPFE, other than supportive care (e.g., smoking cessation).

Diagnosis of Pulmonary Hypertension Associated with Lung Disease

The diagnosis of pulmonary hypertension associated with lung disease (e.g., pulmonary hypertension associated with lung disease (e.g., pulmonary hypertension associated with chronic obstructive pulmonary disease (COPD), interstitial lung disease (ILD), or combined pulmonary fibrosis and emphysema (CPFE)), including functional group, can be determined based on symptoms and physical examination using a review of a comprehensive set of parameters to determine if the hemodynamic and other criteria are met. Some of the criteria which may considered include the patient's clinical presentation (e.g., shortness of breath, fatigue, weakness, angina, syncope, dry-couch, exercise-induced nausea and vomiting), electrocardiogram (ECG) results, chest radiograph results, pulmonary function tests, arterial blood gases, echocardiography results, ventilation/perfusion lung scan results, high-resolution computed tomography results, contrast-enhanced computed tomography results, pulmonary angiography results, cardiac magnetic resonance imaging, blood tests (e.g., biomarkers such as BNP or NT-proBNP), immunology, abdominal ultrasound scan, right heart catheterization (RHC), vasoreactivity, and genetic testing. Galie N., et al Euro Heart J. (2016) 37, 67-119.

A diagnosis of pulmonary hypertension associated with lung disease (Group 3 pulmonary hypertension) is determined when chronic lung disease and/or hypoxemia is present in a person having pulmonary hypertension, and no alternative cause of pulmonary hypertension can be identified. Group 3 pulmonary hypertension can be made based clinical assessments and echocardiographic results, and definitively confirmed by right heart catheterization. Though pulmonary hypertension associated with lung disease has symptomatic and etiological overlap with other types of pulmonary hypertension, several features distinguish this group from the others (e.g., moderate to severe impairment (FEV₁<60% in patients with COPD, and FVC <70% in patients with pulmonary fibrosis), characteristic imaging of a lung disorder or polysomnographic findings of a sleep disorder, reduced breathing reserve, normal oxygen pulse, mixed venous oxygen saturation above the lower limit of normal, and an increase in the partial arterial pressure of carbon dioxide during exercise (especially in COPD), and presence of mild to moderate pulmonary hypertension on echocardiography or right heart catheterization).

### Measurements of Group 3 PH

Various pulmonary hemodynamic parameters are helpful in assessing disease progression and a patient's responsiveness to treatment protocols. Typically, these parameters describe or evaluate the blood flow through the heart and pulmonary vasculature. Examples of pulmonary hemodynamic parameters include, but are not limited to, mean pulmonary artery pressure (mPAP), diastolic pulmonary artery pressure (dPAP) [also known as pulmonary artery diastolic pressure (PADP)], systolic pulmonary artery pressure (sPAP) [also known as pulmonary artery systolic pressure (PASP)], mean right atrial pressure (mRAP), pulmonary capillary wedge pressure (PCWP) [also known as pulmonary artery wedge pressure (PAWP)], pulmonary vascular resistance (PVR) and cardiac output (CO). In certain aspects, the disclosure relates to methods of treating pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1, wherein the method alters or modifies one or more of the following parameters:
a) reduces the right ventricular systolic pressure (RVSP);
b) reduces mPAP
c) reduces mRAP;
d) decreases PVR;
e) decreases the diastolic pressure gradient (DPG);
f) decreases the BNP levels;
g) decreases the NT-proBNP levels;
h) decreases smooth muscle hypertrophy;
i) decreases a patient's CAMPHOR score;
j) improves ventricular function;
k) decreases right ventricular hypertrophy;
l) increases cardiac index;
m) increases the cardiac output;
n) decreases the composite physiologic index;
o) increases the arterial oxygen saturation;
p) increases exercise capacity;
q) increases forced expiratory volume;
r) increases forced vital capacity (FVC);
s) increases the DL_{CO};
t) reduces pulmonary fibrosis; and/or
u) increases transplant free survival in the patient.
   mPAP

Pulmonary blood pressure is normally a lot lower than systemic blood pressure. Normal pulmonary artery pressure is typically between 8-20 mm Hg at rest. If the pressure in the pulmonary artery is greater than 25 mm Hg at rest or 30 mm Hg during physical activity, it is abnormally high and is characterized as pulmonary hypertension.

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the patient's mPAP is reduced by at least 10%. In some embodiments, the method relates to patients having an mPAP of at least 17 mmHg. In some embodiments, the method relates to patients having an mPAP of at least 20 mmHg. In some embodiments, the method relates to patients having an mPAP of at least 25 mmHg. In some embodiments, the method relates to patients having an mPAP between 25-34 mmHg. In some embodiments, the method relates to patients having an mPAP of at least 30 mmHg. In some embodiments, the method relates to patients having an mPAP of at least 35 mmHg. In some embodiments, the method relates to patients having an mPAP of at least 40 mmHg. In some embodiments, the method relates to patients having an mPAP of at least 45 mmHg. In some embodiments, the method relates to patients having an mPAP of at least 50 mmHg.

In some embodiments, the method relates to patients having an mPAP between 21-24 mmHg and a PVR of at least 3 Wood Units. In some embodiments, the method relates to patients having an mPAP of greater than 25 mmHg with a Cardiac Index (CI) of less than 2.0 L/min/m². In some embodiments, the method relates to patients having an mPAP of greater than 25 mmHg with a CI of less than 2.5 L/min/m².

In some embodiments, the method relates to reducing the mPAP in the patient by at least 10% (e.g., 10%, 15%, 20%, 25%, 30%, 35%, 40%, 45%, or at least 50%). In some embodiments, the method relates to reducing the mPAP in the patient by at least 15%. In some embodiments, the method relates to reducing the mPAP in the patient by at least 20%. In some embodiments, the method relates to reducing the mPAP in the patient by at least 25%. In some embodiments, the method relates to reducing the mPAP in the patient by at least 30%. In some embodiments, the method relates to reducing the mPAP in the patient by at least 35%. In some embodiments, the method relates to reducing the mPAP in the patient by at least 40%. In some embodiments, the method relates to reducing the mPAP in the patient by at least 45%. In some embodiments, the method relates to reducing the mPAP in the patient by at least 50%.

In some embodiments, the method relates to reducing the mPAP by at least 3 mmHg in the patient. In some embodiments, the method relates to reducing the mPAP by at least 5 mmHg. In some embodiments, the method relates to reducing the mPAP by at least 7 mmHg. In some embodiments, the method relates to reducing the mPAP by at least 10 mmHg. In some embodiments, the method relates to reducing the mPAP by at least 12 mmHg. In some embodiments, the method relates to reducing the mPAP by at least 15 mmHg. In some embodiments, the method relates to reducing the mPAP by at least 20 mmHg. In some embodiments, the method relates to reducing the mPAP by at least 25 mmHg. In some embodiments, the method relates to reducing the mPAP to less than 17 mmHg. In some embodiments, the method relates to reducing the mPAP to less than 20 mmHg. In some embodiments, the method relates to reducing the mPAP to less than 25 mmHg. In some embodiments, the method relates to reducing the mPAP to less than 30 mmHg.

mRAP

Right atrial pressure (RAP) is the blood pressure in the right atrium of the heart. RAP reflects the amount of blood returning to the heart and the ability of the heart to pump the blood into the arterial system. Normal right atrial pressure is typically between 2 mmHg and 6 mmHg. Elevated right atrial pressure reflects right ventricular (RV) overload and is an established risk factor.

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the patient's mRAP is reduced by at least 10%.

In some embodiments, the patient has a mean right atrial pressure (mRAP) of at least 5 mmHg. In some embodiments, the patient has a mean right atrial pressure (mRAP) of at least 6 mmHg. In some embodiments, the patient has a mean right atrial pressure (mRAP) of at least 8 mmHg. In some embodiments, the patient has a mean right atrial pressure (mRAP) of at least 10 mmHg. In some embodiments, the patient has a mean right atrial pressure (mRAP) of at least 12 mmHg. In some embodiments, the patient has a mean right atrial pressure (mRAP) of at least 14 mmHg. In some embodiments, the patient has a mean right atrial pressure (mRAP) of at least 16 mmHg. In some embodiments, the method improves the mean right atrial pressure (mRAP) in the patient. In some embodiments, the improvement in the mRAP is a reduction in the mRAP.

In some embodiments, the method reduces the mRAP in the patient by at least 10%. In some embodiments, the method reduces the mRAP in the patient by at least 15%. In some embodiments, the method reduces the mRAP in the patient by at least 20%. In some embodiments, the method reduces the mRAP in the patient by at least 25%. In some embodiments, the method relates to reducing the mRAP in the patient by at least 30%. In some embodiments, the method relates to reducing the mRAP in the patient by at least 35%. In some embodiments, the method relates to reducing the mRAP in the patient by at least 40%. In some embodiments, the method relates to reducing the mRAP in the patient by at least 45%. In some embodiments, the method relates to reducing the mRAP in the patient by at least 50%.

In some embodiments, the method reduces the mRAP by at least 1 mmHg. In some embodiments, the method reduces the mRAP by at least 1 mmHg in the patient. In some embodiments, the method reduces the mRAP by at least 2 mmHg in the patient. In some embodiments, the method reduces the mRAP by at least 3 mmHg in the patient. In some embodiments, the method reduces the mRAP by at least 4 mmHg in the patient. In some embodiments, the method reduces the mRAP by at least 5 mmHg in the patient. In some embodiments, the method reduces the mRAP by at least 6 mmHg in the patient. In some embodiments, the method reduces the mRAP by at least 7 mmHg in the patient. In some embodiments, the method reduces the mRAP by at least 8 mmHg in the patient. In some embodiments, the method reduces the mRAP by at least 9 mmHg in the patient. In some embodiments, the method reduces the mRAP by at least 10 mmHg in the patient. In some embodiments, the method reduces the mRAP by at least 11 mmHg in the patient. In some embodiments, the method reduces the mRAP by at least 12 mmHg in the patient. In some embodiments, the method reduces the mRAP by at least 13 mmHg in the patient. In some embodiments, the method reduces the mRAP by at least 14 mmHg in the patient. In some embodiments, the method reduces the mRAP by at least 15 mmHg in the patient.

### PVR

Vascular resistance is the resistance that must be overcome to push blood through the circulatory system and create flow. Pulmonary vascular resistance is the resistance against blood flow from the pulmonary artery to the left atrium. Total blood flow represents the cardiac output (5 to 6 L/min). A normal value for pulmonary vascular resistance using conventional units is 0.25-1.6 mmHg·min/l. Pulmonary vascular resistance can also be represented in units of dynes/sec/cm5 (normal=37-250 dynes/sec/cm5). One of the factors that contributes to an increase in PVR is hypoxemia. In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1, wherein the patient's PVR is decreased by at least 10%.

In some embodiments, the patient has a pulmonary vascular resistance (PVR) greater than or equal to 3 Wood Units. In some embodiments, the method decreases the PVR in the patient. In some embodiments, the method reduces the PVR in the patient by at least 10%. In some embodiments, the method reduces the PVR in the patient by at least 15%. In some embodiments, the method reduces the PVR in the patient by at least 20%. In some embodiments, the method reduces the PVR in the patient by at least 25%. In some embodiments, the method reduces the PVR in the patient by at least 30%. In some embodiments, the method reduces the PVR in the patient by at least 35%. In some embodiments, the method reduces the PVR in the patient by at least 40%. In some embodiments, the method reduces the PVR in the patient by at least 45%. In some embodiments, the method reduces the PVR in the patient by at least 50%. In some embodiments, the method reduces the PVR to less than 3 Woods Units.

### DPG

The pulmonary artery diastolic pressure gradient, DPG, has historically been used to determine the difference between diastolic pulmonary artery pressure and the wedge pressure. In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1 wherein the patient's DPG is decreased by at least 10%.

In some embodiments, the patient has a diastolic pressure gradient (DPG) of greater than 7 mmHg. In some embodiments, the patient has a DPG of at least 7 mmHg. In some embodiments, the patient has a DPG of at least 10 mmHg. In some embodiments, the patient has a DPG of at least 15 mmHg. In some embodiments, the patient has a DPG of at least 20 mmHg. In some embodiments, the patient has a DPG of at least 25 mmHg. In some embodiments, the patient has a DPG of at least 30 mmHg. In some embodiments, the patient has a DPG of at least 35 mmHg. In some embodiments, the patient has a DPG of at least 40 mmHg. In some embodiments, the patient has a DPG of at least 45 mmHg. In some embodiments, the patient has a DPG of at least 50 mmHg.

In some embodiments, the method decreases the DPG in the patient. In some embodiments, the method decreases the DPG in the patient by at least 10%. In some embodiments, the method decreases the DPG in the patient by at least 15%. In some embodiments, the method decreases the DPG in the patient by at least 20%. In some embodiments, the method decreases the DPG in the patient by at least 25%. In some embodiments, the method decreases the DPG in the patient by at least 30%. In some embodiments, the method decreases the DPG in the patient by at least 35%. In some embodiments, the method decreases the DPG in the patient by at least 40%. In some embodiments, the method decreases the DPG in the patient by at least 45%. In some embodiments, the method decreases the DPG in the patient by at least 50%. In some embodiments, the method decreases the DPG in the patient to less than 7 mmHg.

### BNP

Brain natriuretic peptide (BNP) and NT-proBNP are markers of atrial and ventricular distension due to increased intracardiac pressure. The New York Heart Association (NYHA) developed a 4-stage functional classification system for congestive heart failure (CHF) based on the severity of symptoms. Studies have demonstrated that the measured concentrations of circulating BNP and NT-proBNP increase with the severity of CHF based on the NYHA classification. In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1, wherein the patient's BNP levels are decreased.

In some embodiments, the patient has elevated BNP levels as compared to a healthy patient (e.g., a healthy person of similar age and sex). In some embodiments, the patient has normal BNP levels. In some embodiments, the patient has a BNP level of at least 100 pg/mL. In some embodiments, the patient has a BNP level of at least 150 pg/mL. In some embodiments, the patient has a BNP level of at least 200 pg/mL. In some embodiments, the patient has a BNP level of at least 300 pg/mL. In some embodiments, the patient has a BNP level of at least 400 pg/mL. In some embodiments, the patient has a BNP level of at least 500 pg/mL. In some embodiments, the patient has a BNP level of at least 1000 pg/mL. In some embodiments, the patient has a BNP level of at least 3000 pg/mL. In some embodiments, the patient has a BNP level of at least 5000 pg/mL. In some embodiments, the patient has a BNP level of at least 10,000 pg/mL. In some embodiments, the patient has a BNP level of at least 15,000 pg/mL. In some embodiments, the patient has a BNP level of at least 20,000 pg/mL.

In some embodiments, the method decreases BNP levels in the patient by at least 10%. In some embodiments, the method decreases BNP levels in the patient by at least 20%. In some embodiments, the method decreases BNP levels in the patient by at least 25%. In some embodiments, the method decreases BNP levels in the patient by at least 30%. In some embodiments, the method decreases BNP levels in the patient by at least 35%. In some embodiments, the method decreases BNP levels in the patient by at least 40%. In some embodiments, the method decreases BNP levels in the patient by at least 45%. In some embodiments, the method decreases BNP levels in the patient by at least 50%. In some embodiments, the method decreases BNP levels in the patient by at least 55%. In some embodiments, the method decreases BNP levels in the patient by at least 60%. In some embodiments, the method decreases BNP levels in the patient by at least 65%. In some embodiments, the method decreases BNP levels in the patient by at least 70%. In some embodiments, the method decreases BNP levels in the patient by at least 75%. In some embodiments, the method decreases BNP levels in the patient by at least 80%. In some embodiments, the method decreases BNP levels to normal levels (i.e., <100 pg/ml).

### NT-proBNP

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1 wherein the patient's NT-proBNP levels are decreased.

In some embodiments, the patient has elevated NT-proBNP levels as compared to a healthy patient (e.g., a healthy person of similar age and sex). In some embodiments, the patient has normal NT-proBNP levels. In some embodiments, the patient has a NT-proBNP level of at least 100 pg/mL. In some embodiments, the patient has a NT-proBNP level of at least 150 pg/mL. In some embodiments, the patient has a NT-proBNP level of at least 200 pg/mL. In some embodiments, the patient has a NT-proBNP level of at least 300 pg/mL. In some embodiments, the patient has a NT-proBNP level of at least 400 pg/mL. In some embodiments, the patient has a NT-proBNP level of at least 500 pg/mL. In some embodiments, the patient has a NT-proBNP level of at least 1000 pg/mL. In some embodiments, the patient has a NT-proBNP level of at least 3000 pg/mL. In some embodiments, the patient has a NT-proBNP level of at least 5000 pg/mL. In some embodiments, the patient has a NT-proBNP level of at least 10,000 pg/mL. In some embodiments, the patient has a NT-proBNP level of at least 15,000 pg/mL. In some embodiments, the patient has a NT-proBNP level of at least 20,000 pg/mL.

In some embodiments, the method decreases NT-proBNP levels in the patient. In some embodiments, the method decreases NT-proBNP levels in the patient by at least 10%. In some embodiments, the method decreases NT-proBNP levels in the patient by at least 20%. In some embodiments, the method decreases NT-proBNP levels in the patient by at least 25%. In some embodiments, the method decreases NT-proBNP levels in the patient by at least 30%. In some embodiments, the method decreases NT-proBNP levels in the patient by at least 35%. In some embodiments, the method decreases NT-proBNP levels in the patient by at least 40%. In some embodiments, the method decreases NT-proBNP levels in the patient by at least 45%. In some embodiments, the method decreases NT-proBNP levels in the patient by at least 50%. In some embodiments, the method decreases NT-proBNP levels in the patient by at least 55%. In some embodiments, the method decreases NT-proBNP levels in the patient by at least 60%. In some embodiments, the method decreases NT-proBNP levels in the patient by at least 65%. In some embodiments, the method decreases NT-proBNP levels in the patient by at least 70%. In some embodiments, the method decreases NT-proBNP levels in the patient by at least 75%. In some embodiments, the method decreases NT-proBNP levels in the patient by at least 80%. In some embodiments, the method decreases NT-proBNP levels in the patient by at least 30%. In some embodiments, the method decreases NT-proBNP levels to normal levels. In some embodiments, the normal level of NT-proBNP is <100 pg/ml.

### Smooth Muscle Hypertrophy

Patients with COPD often experience airway wall remodeling, mostly in small airways, leading to airway wall thickening and airflow obstruction. Similarly, bronchial smooth muscle hypertrophy, characterized by an increase in the smooth muscle cells and thickening of the smooth muscle layer around airways, is a feature of airway wall remodeling in disease states resembling chronic asthma. In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1, wherein the method decreases smooth muscle hypertrophy.

In some embodiments, the method decreases smooth muscle hypertrophy in the patient. In some embodiments, the method decreases smooth muscle hypertrophy in the patient by at least 10%. In some embodiments, the method decreases smooth muscle hypertrophy in the patient by at least 15%. In some embodiments, the method decreases smooth muscle hypertrophy in the patient by at least 20%. In some embodiments, the method decreases smooth muscle hypertrophy in the patient by at least 25%. In some embodiments, the method decreases smooth muscle hypertrophy in the patient by at least 30%. In some embodiments, the method decreases smooth muscle hypertrophy in the patient by at least 35%. In some embodiments, the method decreases smooth muscle hypertrophy in the patient by at least 40%. In some embodiments, the method decreases smooth muscle hypertrophy in the patient by at least 45%. In some embodiments, the method decreases smooth muscle hypertrophy in the patient by at least 50%.

### Quality of Life

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the method improves a patient's quality of life.

In some embodiments, the patient's quality of life is measured using the Cambridge Pulmonary Hypertension Outcome Review (CAMPHOR). CAMPHOR is a disease specific patient-reported outcome measure which assesses the quality of life of patients with pulmonary hypertension. There are three dimension of CAMPHOR which assess symptoms, functioning, and quality of life. The quality of life (QoL) scale has twenty-five items which focus on socialization, role, acceptance, self-esteem, independence, and security. Similarly, the symptom dimension consists of twenty-five symptoms broken into three subscales: energy, breathlessness, and mood. The activity scale has fifteen items. Scores for QoL and symptoms range from 0-25, with higher scores indicating worse quality of life. Activity scores range from 0-30, with higher scores indicating more physical limitations. In some embodiments, the method decreases the patient's quality of life (QoL) score by at least 1%. In some embodiments, the method decreases the patient's quality of life (QoL) score by at least 2%. In some embodiments, the method decreases the patient's quality of life (QoL) score by at least 3%. In some embodiments, the method decreases the patient's quality of life (QoL) score by at least 4%. In some embodiments, the method decreases the patient's quality of life (QoL) score by at least 5%. In some embodiments, the method decreases the patient's quality of life (QoL) score by at least 10%. In some embodiments, the method decreases the patient's quality of life (QoL) score by at least 15%. In some embodiments, the method decreases the patient's quality of life (QoL) score by at least 20%. In some embodiments, the method decreases the patient's quality of life (QoL) score by at least 25%. In some embodiments, the method decreases the patient's quality of life (QoL) score by at least 30%. In some embodiments, the method decreases the patient's quality of life (QoL) score by at least 35%. In some embodiments, the method decreases the patient's quality of life (QoL) score by at least 40%. In some embodiments, the method decreases the patient's quality of life (QoL) score by at least 45%. In some embodiments, the method decreases the patient's quality of life (QoL) score by at least 50%. In some embodiments, the method decreases the patient's quality of life (QoL) score by at least 55%. In some embodiments, the method decreases the patient's quality of life (QoL) score by at least 60%. In some embodiments, the method decreases the patient's quality of life (QoL) score by at least 65%. In some embodiments, the method decreases the patient's quality of life (QoL) score by at least 70%. In some embodiments, the method decreases the patient's quality of life (QoL) score by at least 75%. In some embodiments, the method decreases the patient's quality of life (QoL) score by at least 80%. In some embodiments, the method decreases the patient's quality of life (QoL) score by at least 85%. In some embodiments, the method decreases the patient's quality of life (QoL) score by at least 90%. In some embodiments, the method decreases the patient's quality of life (QoL) score by at least 95%. In some embodiments, the method decreases the patient's quality of life (QoL) score by at least 100%. In some embodiments, the patient's quality of life is improved as measured using the Cambridge Pulmonary Hypertension Outcome Review (CAMPHOR).

### Ventricular Function

In certain aspects, the disclosure relates to methods of improving or maintaining ventricular function (e.g., left ventricular function or right ventricular function). In some embodiments, the method improves right ventricular function in the patient. In some embodiments, the improvement in right ventricular function is due to an increase in right ventricular fractional area change. In some embodiments, the improvement in right ventricular function is due to a decrease in right ventricular hypertrophy. In some embodiments, the improvement in ejection fraction. In some embodiments, the improvement in the right ventricular hypertrophy in the patient.

In certain aspects, the disclosure relates to diagnostic tests and methods for pulmonary hypertension associated with lung disease (e.g., pulmonary hypertension associated with chronic obstructive pulmonary disease (COPD), interstitial lung disease (ILD), or combined pulmonary fibrosis and emphysema (CPFE)). Echocardiography is a useful noninvasive screening tool for determining the severity of pulmonary hypertension in a patient. Improvement or maintenance of ventricular function (e.g., left ventricular function or right ventricular function) can be assessed by many echocardiographic measurements. One such quantitative approach to assess ventricular function is the measurement of the tricuspid annular plane systolic excursion (TAPSE). The TAPSE estimates RV systolic function by measuring the level of systolic excursion of the lateral tricuspid valve annulus towards the apex. Other echocardiographic measurements that may be used to assess maintenance and/or improvements in ventricular function include, but are not limited to, right ventricular fractional area change (RVFAC), right ventricular end-diastolic area (RVEDA), right ventricular end-systolic area (RVESA), right ventricular free wall thickness (RVFWT), right ventricular ejection fraction (RVEF), right ventricular-pulmonary artery (RV-PA) coupling, pulmonary arterial systolic pressure (PASP), right ventricular systolic pressure (RVSP), pulmonary artery acceleration time (PAAT), tricuspid regurgitation velocity (TRV), left ventricular hypertrophy, and right ventricular hypertrophy.

### TAPSE

The tricuspid annular plane systolic excursion (TAPSE) can be obtained using echocardiography and represents a measure of RV longitudinal function. The TAPSE has previously been shown to have good correlations with parameters estimating RV global systolic function. A TAPSE <17 mm is highly suggestive of RV systolic dysfunction. In some embodiments of the methods disclosed herein, the patient has a TAPSE of less than 20 mm. In some embodiments, the patient has a TAPSE of less than 18 mm. In some embodiments, the patient has a TAPSE of less than 16 mm. In some embodiments, the patient has a TAPSE of less than 14 mm. In some embodiments, the patient has a TAPSE of less than 12 mm.

In some embodiments, the method increases the TAPSE to at least 20 mm. In some embodiments, the method increases the TAPSE to at least 22 mm. In some embodiments, the method increases the TAPSE to at least 24 mm. In some embodiments, the method increases the TAPSE to at least 26 mm. In some embodiments, the method increases the TAPSE to at least 28 mm. In some embodiments, the method increases the TAPSE to at least 30 mm.

### PASP and RVSP

In certain aspects, the disclosure relates to methods of treating pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the method reduces the right ventricular systolic pressure (RVSP) by at least 10%.

In some embodiments, the PASP is a resting PASP. In some embodiments, the PASP is determined using the tricuspid regurgitation velocity (TRV) and right arterial (RA) pressure.

In some embodiments, the PASP is determined using the following formula:

$$P A S P = T R {V}^{2} \times 4 + R A pressure$$

TRV has been shown to correlate with PASP at rest and with exercise. The pressure gradient between the right ventricle and the right atrium can be calculated using the modified Bernoulli equation (Δp=4V²).

In some embodiments, the right ventricular systolic pressure (RVSP) is equal to PASP. In some embodiments, the RVSP is measured in the absence of right ventricular outflow tract obstruction. In some embodiments, the RVSP is determined using the following formula:

$$R V S P = 4 {V}^{2} + R A P$$

In the above formula, V represents the peak tricuspid regurgitant jet velocity and RAP is the mean right atrial pressure. RVSP is frequently used for estimating PASP.

In some embodiments, the patient has a right ventricular systolic pressure (RVSP) of greater than 35 mmHg. In some embodiments, the method decreases the RVSP in the patient. In some embodiments, the methods reduce the RVSP in the patient by at least 10%. In some embodiments, the method decreases the RVSP in the patient by at least 15%. In some embodiments, the methods reduce the RVSP in the patient by at least 20%. In some embodiments, the method decreases the RVSP in the patient by at least 25%. In some embodiments, the methods reduce the RVSP in the patient by at least 30%. In some embodiments, the method decreases the RVSP in the patient by at least 35%. In some embodiments, the methods reduce the RVSP in the patient by at least 40%. In some embodiments, the method decreases the RVSP in the patient by at least 45%. In some embodiments, the methods reduce the RVSP in the patient by at least 50%. In some embodiments, the methods reduce the RVSP in the patient to less than 25 mmHg.

In some embodiments, the patient has a pulmonary artery systolic pressure (PASP) of greater than 20 mmHg. In some embodiments, the patient has a PASP of greater than 25 mmHg. In some embodiments, the patient has a PASP of at least 35 mmHg. In some embodiments, the patient has a PASP of at least 40 mmHg. In some embodiments, the patient has a PASP of at least 50 mmHg. In some embodiments, the patient has a PASP of at least 55 mmHg. In some embodiments, the patient has a PASP of at least 60 mmHg.

In some embodiments, the method decreases the PASP in the patient. In some embodiments, the method reduces the PASP in the patient by at least 10%. In some embodiments, the method decreases the PASP in the patient by at least 15%. In some embodiments, the methods reduce the PASP in the patient by at least 20%. In some embodiments, the method decreases the PASP in the patient by at least 25%. In some embodiments, the methods reduce the PASP in the patient by at least 30%. In some embodiments, the method decreases the PASP in the patient by at least 35%. In some embodiments, the methods reduce the PASP in the patient by at least 40%. In some embodiments, the method decreases the PASP in the patient by at least 45%. In some embodiments, the methods reduce the PASP in the patient by at least 50%.

In some embodiments, the method reduces the PASP in the patient by at least 5 mmHg. In some embodiments, the method reduces the PASP in the patient by at least 10 mmHg. In some embodiments, the method reduces the PASP in the patient by at least 15 mmHg. In some embodiments, the method reduces the PASP in the patient by at least 20 mmHg. In some embodiments, the method reduces the PASP in the patient by at least 25 mmHg. In some embodiments, the method reduces the PASP in the patient to less than 25 mmHg. In some embodiments, the method reduces the PASP in the patient to less than 20 mmHg.

### Right Ventricular Hypertrophy

Right ventricular hypertrophy (RVH) is a pathological increase in muscle mass of the right ventricle in response to pressure overload, most commonly due to severe lung disease. Symptoms of RVH due to pulmonary hypertension include exertional chest pain, peripheral edema, exertional syncope, and right upper quadrant pain. In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80% b, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1.

In some embodiments, the methods decrease right ventricular hypertrophy in the patient. In some embodiments, the methods decrease right ventricular hypertrophy by at least 10%. In some embodiments, the methods decrease right ventricular hypertrophy by at least 15%. In some embodiments, the methods decrease right ventricular hypertrophy by at least 20%. In some embodiments, the methods decrease right ventricular hypertrophy by at least 25%. In some embodiments, the methods decrease right ventricular hypertrophy by at least 30%. In some embodiments, the methods decrease right ventricular hypertrophy by at least 35%. In some embodiments, the methods decrease right ventricular hypertrophy by at least 40%. In some embodiments, the methods decrease right ventricular hypertrophy by at least 45%. In some embodiments, the methods decrease right ventricular hypertrophy by at least 50%.

### Cardiac Index

Cardiac index (CI) is an assessment of cardiac output based on a patient's size. Both the cardiac output and cardiac index are important in determining whether the heart is pumping enough blood and delivering enough oxygen to cells. Cardiac index allows the comparison of cardiac function between individuals of different sizes. In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1, wherein the method increases cardiac index.

In some embodiments, the patient has a cardiac index of less than 2.5 L/min/m². In some embodiments, the patient has a cardiac index of less than 2.0 L/min/m². In some embodiments, the patient has a cardiac index of less than 1.5 L/min/m². In some embodiments, the patient has a cardiac index of less than 1.0 L/min/m². In some embodiments, the method increases the CI in the patient by at least 10%. In some embodiments, the method increases the CI in the patient by at least 10%. In some embodiments, the method increases the CI in the patient by at least 10%. In some embodiments, the method increases the CI in the patient by at least 15%. In some embodiments, the method increases the CI in the patient by at least 20%. In some embodiments, the method increases the CI in the patient by at least 25%. In some embodiments, the method increases the CI in the patient by at least 30%. In some embodiments, the method increases the CI in the patient by at least 35%. In some embodiments, the method increases the CI in the patient by at least 40%. In some embodiments, the method increases the CI in the patient by at least 45%. In some embodiments, the method increases the CI in the patient by at least 50%. In some embodiments, the method increases the CI in the patient by at least 0.2 L/min/m². In some embodiments, the method increases the CI in the patient by at least 0.4 L/min/m². In some embodiments, the method increases the CI in the patient by at least 0.6 L/min/m². In some embodiments, the method increases the CI in the patient by at least 0.8 L/min/m². In some embodiments, the method increases the CI in the patient by at least 1 L/min/m². In some embodiments, the method increases the CI in the patient by at least 1.2 L/min/m². In some embodiments, the method increases the CI in the patient by at least 1.4 L/min/m². In some embodiments, the method increases the CI in the patient by at least 1.6 L/min/m². In some embodiments, the method increases the CI in the patient by at least 1.8 L/min/m². In some embodiments, the method increases the CI in the patient by at least 2 L/min/m². In some embodiments, the method increases the CI in the patient to at least 2.5 L/min/m².

### Cardiac Output

In general, normal cardiac output at rest is about 2.5-4.2 L/min/m2, and cardiac output can decline by almost 40% without deviating from the normal limits. A low cardiac index of less than about 2.5 L/min/m2 usually indicates a disturbance in cardiovascular performance. The cardiac output can be utilized to calculate the cardiac index (e.g., cardiac index=cardiac output/body surface area). The cardiac output can be also utilized to calculate the stroke volume (e.g., stroke volume=CO/heart rate). In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1, wherein the method increases the cardiac output.

In some embodiments, the patient has a cardiac output of less than 4 L/min. In some embodiments, the method increases the cardiac output in the patient by at least 10%. In some embodiments, the method increases the cardiac output in the patient by at least 15%. In some embodiments, the method increases the cardiac output in the patient by at least 20%. In some embodiments, the method increases the cardiac output in the patient by at least 25%. In some embodiment, the method increases the cardiac output in the patient by at least 30%. In some embodiments, the method increases the cardiac output in the patient by at least 35%. In some embodiments, the method increases the cardiac output in the patient by at least 40%. In some embodiments, the method increases the cardiac output in the patient by at least 45%. In some embodiments, the method increases the cardiac output in the patient by at least 50%. In some embodiments, the method increases the cardiac output in the patient by at least 0.5 L/min. In some embodiments, the method increases the cardiac output in the patient by at least 1 L/min. In some embodiments, the method increases the cardiac output in the patient by at least 1.5 L/min. In some embodiments, the method increases the cardiac output in the patient by at least 2 L/min. In some embodiments, the method increases the cardiac output in the patient by at least 2.5 L/min. In some embodiments, the method increases the cardiac output in the patient by at least 3 L/min. In some embodiments, the method increases the cardiac output in the patient by at least 3.5 L/min. In some embodiments, the method increases the cardiac output in the patient by at least 4 L/min.

### Composite Physiologic Index (CPI)

Composite physiologic index (CPI) can be used to determine the extent of pulmonary fibrosis. It is difficult to predict the clinical course of fibrotic lung diseases (e.g., idiopathic pulmonary fibrosis). CPI models can be used as a predictor of fibrotic disease progression. In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the method decreases the composite physiologic index.

In some embodiments, the patient has a CPI greater than 15. In some embodiments of the methods herein, the patient has a CPI greater than 20. In some embodiments of the methods herein, the patient has a CPI greater than 25. In some embodiments of the methods herein, the patient has a CPI greater than 30. In some embodiments of the methods herein, the patient has a CPI greater than 35. In some embodiments of the methods herein, the patient has a CPI greater than 40. In some embodiments of the methods herein, the patient has a CPI greater than 45. In some embodiments of the methods herein, the patient has a CPI greater than 50. In some embodiments of the methods herein, the patient has a CPI greater than 55. In some embodiments of the methods herein, the patient has a CPI greater than 60. In some embodiments of the methods herein, the patient has a CPI greater than 65. In some embodiments of the methods herein, the patient has a CPI greater than 70. In some embodiments of the methods herein, the patient has a CPI greater than 75. In some embodiments of the methods herein, the patient has a CPI greater than 80. In some embodiments, the method decreases the CPI in the patient. In some embodiments, the method decreases the CPI in the patient by 10%. In some embodiments, the method decreases the CPI in the patient by 15%. In some embodiments, the method decreases the CPI in the patient by 20%. In some embodiments, the method decreases the CPI in the patient by 25%. In some embodiments, the method decreases the CPI in the patient by 30%. In some embodiments, the method decreases the CPI in the patient by 35%. In some embodiments, the method decreases the CPI in the patient by 40%. In some embodiments, the method decreases the CPI in the patient by 45%. In some embodiments, the method decreases the CPI in the patient by 50%. In some embodiments, the method decreases the CPI to less than 70. In some embodiments, the method decreases the CPI to less than 65. In some embodiments, the method decreases the CPI to less than 60. In some embodiments, the method decreases the CPI to less than 55. In some embodiments, the method decreases the CPI to less than 50. In some embodiments, the method decreases the CPI to less than 45. In some embodiments, the method decreases the CPI to less than 40. In some embodiments, the method decreases the CPI to less than 35. In some embodiments, the method decreases the CPI to less than 30. In some embodiments, the method decreases the CPI to less than 25. In some embodiments, the method decreases the CPI to less than 20. In some embodiments, the method decreases the CPI to less than 15. In some embodiments, the method decreases the CPI to less than 10. In some embodiments, the method decreases the CPI to less than 5.

### Oxygen Saturation at Rest

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the method increases the arterial oxygen saturation.

In some embodiments, the patient has an arterial oxygen saturation of less than 95%. In some embodiments of the methods disclosed herein, the patient has an arterial oxygen saturation of less than 90%. In some embodiments of the methods disclosed herein, the patient has an arterial oxygen saturation of less than 85%. In some embodiments of the methods disclosed herein, the patient has an arterial oxygen saturation of less than 80%. In some embodiments of the methods disclosed herein, the patient has an arterial oxygen saturation of less than 75%. In some embodiments of the methods disclosed herein, the patient has an arterial oxygen saturation of less than 70%. In some embodiments of the methods disclosed herein, the patient has an arterial oxygen saturation of less than 65%. In some embodiments of the methods disclosed herein, the patient has an arterial oxygen saturation of less than 60%. In some embodiments of the methods disclosed herein, the patient has an arterial oxygen saturation of less than 55%. In some embodiments of the methods disclosed herein, the patient has an arterial oxygen saturation of less than 50%. In some embodiments of the methods disclosed herein, the patient has an arterial oxygen saturation of less than 45%. In some embodiments of the methods disclosed herein, the patient has an arterial oxygen saturation of less than 40%. In some embodiments of the methods disclosed herein, the patient has an arterial oxygen saturation of less than 35%. In some embodiments of the methods disclosed herein, the patient has an arterial oxygen saturation of less than 30%. In some embodiments, the method increases the arterial oxygen saturation in a patient. In some embodiments, the method increases the arterial oxygen saturation in the patient by at least 5%.

In some embodiments, the method increases the arterial oxygen saturation in the patient by at least 10%. In some embodiments, the method increases the arterial oxygen saturation in the patient by at least 15%. In some embodiments, the method increases the arterial oxygen saturation in the patient by at least 20%. In some embodiments, the method increases the arterial oxygen saturation in the patient by at least 25%. In some embodiments, the method increases the arterial oxygen saturation in the patient by at least 30%. In some embodiments, the method increases the arterial oxygen saturation in the patient by at least 35%. In some embodiments, the method increases the arterial oxygen saturation in the patient by at least 40%. In some embodiments, the method increases the arterial oxygen saturation in the patient by at least 45%. In some embodiments, the method increases the arterial oxygen saturation in the patient by at least 50%. In some embodiments, the method increases the arterial oxygen saturation in the patient by at least 85%. In some embodiments, the method increases the arterial oxygen saturation in the patient by at least 90%. In some embodiments, the method increases the arterial oxygen saturation in the patient by at least 95%. In some embodiments, the arterial oxygen saturation is measured at rest.

### Exercise Capacity (6MWD AND BDI)

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the methods increase exercise capacity in the patient.

Any suitable measure of exercise capacity can be used. For example, exercise capacity in a 6-minute walk test (6MWT), which measures how far the subject can walk in 6 minutes, i.e., the 6-minute walk distance (6MWD), is frequently used to assess pulmonary hypertension severity and disease progression. The BDI is a numerical scale for assessing perceived dyspnea (breathing discomfort), and may be used to measure exercise capacity. It measures the degree of breathlessness, for example, after completion of the 6MWT, where a BDI of 0 indicates no breathlessness and 10 indicates maximum breathlessness. In some embodiments, the patient has a 6-minute walk distance (6MWD) of less than 550 meters. In some embodiments, the patient has a 6-minute walk distance (6MWD) of less than 550 meters. In some embodiments, the patient has a 6-minute walk distance (6MWD) of less than 500 meters. In some embodiments, the patient has a 6-minute walk distance (6MWD) of less than 450 meters. In some embodiments, the patient has a 6-minute walk distance (6MWD) of less than 400 meters. In some embodiments, the patient has a 6-minute walk distance (6MWD) of less than 350 meters. In some embodiments, the patient has a 6-minute walk distance (6MWD) of less than 300 meters. In some embodiments, the patient has a 6-minute walk distance (6MWD) of less than 250 meters. In some embodiments, the patient has a 6-minute walk distance (6MWD) of less than 200 meters. In some embodiments, the patient has a 6-minute walk distance (6MWD) of less than 150 meters. In some embodiments, the method increases the patient's 6MWD by at least 10 meters. In some embodiments, the method increases the patient's 6MWD by at least 15 meters. In some embodiments, the method increases the patient's 6MWD by at least 20 meters. In some embodiments, the method increases the patient's 6MWD by at least 25 meters. In some embodiments, the method increases the patient's 6MWD by at least 30 meters. In some embodiments, the method increases the patient's 6MWD by at least 35 meters. In some embodiments, the method increases the patient's 6MWD by at least 40 meters. In some embodiments, the method increases the patient's 6MWD by at least 45 meters. In some embodiments, the method increases the patient's 6MWD by at least 50 meters. In some embodiments, the method increases the patient's 6MWD by at least 55 meters. In some embodiments, the method increases the patient's 6MWD by at least 60 meters. In some embodiments, the method increases the patient's 6MWD by at least 65 meters. In some embodiments, the method increases the patient's 6MWD by at least 70 meters. In some embodiments, the method increases the patient's 6MWD by at least 75 meters. In some embodiments, the method increases the patient's 6MWD by at least 80 meters. In some embodiments, the method increases the patient's 6MWD by at least 85 meters. In some embodiments, the method increases the patient's 6MWD by at least 90 meters. In some embodiments, the method increases the patient's 6MWD by at least 95 meters. In some embodiments, the method increases the patient's 6MWD by at least 100 meters. In some embodiments, the method increases the patient's 6MWD by at least 125 meters. In some embodiments, the method increases the patient's 6MWD by at least 150 meters. In some embodiments, the method increases the patient's 6MWD by at least 175 meters. In some embodiments, the method increases the patient's 6MWD by at least 200 meters. In some embodiments, the method increases the patient's 6MWD by at least 250 meters. In some embodiments, the method increases the patient's 6MWD by at least 300 meters. In some embodiments, the method increases the patient's 6MWD by at least 400 meters.

In some embodiments, the method increases exercise capacity of the patient. In some embodiments, the patient has a Borg dyspnea index (BDI) at least 0.5 index points. In some embodiments, the patient has a Borg dyspnea index (BDI) at least 1 index points. In some embodiments, the patient has a Borg dyspnea index (BDI) at least 1.5 index points. In some embodiments, the patient has a Borg dyspnea index (BDI) at least 2 index points. In some embodiments, the patient has a Borg dyspnea index (BDI) at least 2.5 index points. In some embodiments, the patient has a Borg dyspnea index (BDI) at least 3 index points. In some embodiments, the patient has a Borg dyspnea index (BDI) at least 3.5 index points. In some embodiments, the patient has a Borg dyspnea index (BDI) at least 4 index points. In some embodiments, the patient has a Borg dyspnea index (BDI) at least 4.5 index points. In some embodiments, the patient has a Borg dyspnea index (BDI) at least 5 index points. In some embodiments, the patient has a Borg dyspnea index (BDI) at least 5.5 index points. In some embodiments, the patient has a Borg dyspnea index (BDI) at least 6 index points. In some embodiments, the patient has a Borg dyspnea index (BDI) at least 6.5 index points. In some embodiments, the patient has a Borg dyspnea index (BDI) at least 7 index points. In some embodiments, the patient has a Borg dyspnea index (BDI) at least 7.5 index points. In some embodiments, the patient has a Borg dyspnea index (BDI) at least 8 index points. In some embodiments, the patient has a Borg dyspnea index (BDI) at least 8.5 index points. In some embodiments, the patient has a Borg dyspnea index (BDI) at least 9 index points. In some embodiments, the patient has a Borg dyspnea index (BDI) at least 9.5 index points. In some embodiments, the patient has a Borg dyspnea index (BDI) at least 10 index points. In some embodiments, the method reduces the patient's Borg dyspnea index (BDI). In some embodiments, the method reduces the patient's BDI by at least 0.5 index points. In some embodiments, the method reduces the patient's BDI by at least 1 index points. In some embodiments, the method reduces the patient's BDI by at least 1.5 index points. In some embodiments, the method reduces the patient's BDI by at least 2 index points. In some embodiments, the method reduces the patient's BDI by at least 2.5 index points. In some embodiments, the method reduces the patient's BDI by at least 3 index points. In some embodiments, the method reduces the patient's BDI by at least 3.5 index points. In some embodiments, the method reduces the patient's BDI by at least 4 index points. In some embodiments, the method reduces the patient's BDI by at least 4.5 index points. In some embodiments, the method reduces the patient's BDI by at least 5 index points. In some embodiments, the method reduces the patient's BDI by at least 5.5 index points. In some embodiments, the method reduces the patient's BDI by at least 6 index points. In some embodiments, the method reduces the patient's BDI by at least 6.5 index points. In some embodiments, the method reduces the patient's BDI by at least 7 index points. In some embodiments, the method reduces the patient's BDI by at least 7.5 index points. In some embodiments, the method reduces the patient's BDI by at least 8 index points. In some embodiments, the method reduces the patient's BDI by at least 8.5 index points. In some embodiments, the method reduces the patient's BDI by at least 9 index points. In some embodiments, the method reduces the patient's BDI by at least 9.5 index points. In some embodiments, the method reduces the patient's BDI by at least 10 index points.

### Pulmonary Function Test

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the methods improve pulmonary function of the patient.

Spirometry, or measuring of breath, is a major pulmonary function test that can determine volume and/or speed (flow) of air that is inhaled and exhaled by a subject. A spirometer is used to measure forced vital capacity (FVC) (measured in liters and/or percentage of predicted) in a forced expiratory volume (FEV) test, among other characteristics. In an FEV test, a subject takes a deep breath, and exhales into a sensor as hard and for as long as possible (e.g., at least 6 seconds). Inhalation can also be tested using spirometry. An FEV test is typically repeated at least three times to ensure accuracy. “Normal” ranges for FVC are typically considered to be between 80% and 100% of predicted. “Of predicted” refers to reporting the subject's results as a percentage of the known predicted values for a healthy subject of similar characteristics (e.g., height, sex, age, race, weight). Other measurements that can be taken include, but are not limited to, FEV₁, wherein the FVC is measured within the first second of forced exhalation, and/or forced expiratory flow (FEF), which measures the flow of air coming out of the lung during the middle portion of forced expiration. An FEV₁/FVC ratio is also typically calculated.

In some embodiments of the methods disclosed herein, the patient has a forced expiratory volume in one second (FEV₁) of greater than 70%. In some embodiments of the methods disclosed herein, the patient has a forced expiratory volume in one second (FEV₁) of 60% to 69%. In some embodiments of the methods disclosed herein, the patient has a forced expiratory volume in one second (FEV₁) of 50% to 59%. In some embodiments of the methods disclosed herein, the patient has a forced expiratory volume in one second (FEV₁) of 35% to 49%. In some embodiments of the methods disclosed herein, the patient has a forced expiratory volume in one second (FEV₁) of less than 35%. In some embodiments, the method increases the FEV₁ in the patient. In some embodiments, the method increases the FEV₁ in the patient by at least 5%. In some embodiments, the method increases the FEV₁ in the patient by at least 10%. In some embodiments, the method increases the FEV₁ in the patient by at least 15%. In some embodiments, the method increases the FEV₁ in the patient by at least 20%. In some embodiments, the method increases the FEV₁ in the patient by at least 25%. In some embodiments, the method increases the FEV₁ in the patient by at least 30%. In some embodiments, the method increases the FEV₁ in the patient by at least 35%. In some embodiments, the method increases the FEV₁ in the patient by at least 40%. In some embodiments, the method increases the FEV₁ in the patient by at least 45%. In some embodiments, the method increases the FEV₁ in the patient by at least 50%. In some embodiments, the method increases the FEV₁ to at least 60%. In some embodiments, the method increases the FEV₁ to at least 65%. In some embodiments, the method increases the FEV₁ to at least 70%. In some embodiments, the method increases the FEV₁ to at least 75%. In some embodiments, the method increases the FEV₁ to at least 80%. In some embodiments, the method increases the FEV₁ to at least 85%. In some embodiments, the method increases the FEV₁ to at least 90%. In some embodiments, the method increases the FEV₁ to at least 95%.

In some embodiments, the patient has a forced vital capacity (FVC) of greater than 80%. In some embodiments, the patient has a forced vital capacity (FVC) of greater than 70%. In some embodiments, the patient has a forced vital capacity (FVC) of 60% to 69%. In some embodiments, the patient has a forced vital capacity (FVC) of 50% to 59%. In some embodiments, the patient has a forced vital capacity (FVC) of 35% to 49%. In some embodiments, the patient has a forced vital capacity (FVC) of less than 35%.

In some embodiments, the method increases the FVC in the patient. In some embodiments, the method increases the FVC in the patient by at least 5%. In some embodiments, the method increases the FVC in the patient by at least 10%. In some embodiments, the method increases the FVC in the patient by at least 15%. In some embodiments, the method increases the FVC in the patient by at least 20%. In some embodiments, the method increases the FVC in the patient by at least 25%. In some embodiments, the method increases the FVC in the patient by at least 30%. In some embodiments, the method increases the FVC in the patient by at least 35%. In some embodiments, the method increases the FVC in the patient by at least 40%. In some embodiments, the method increases the FVC in the patient by at least 45%. In some embodiments, the method increases the FVC in the patient by at least 50%. In some embodiments, the method increases the FVC to at least 60%. In some embodiments, the method increases the FVC to at least 65%. In some embodiments, the method increases the FVC to at least 70%. In some embodiments, the method increases the FVC to at least 75%. In some embodiments, the method increases the FVC to at least 80%. In some embodiments, the method increases the FVC to at least 85%. In some embodiments, the method increases the FVC to at least 90%. In some embodiments, the method increases the FVC to at least 95%.

### Carbon Monoxide Transfer Coefficient K_{CO}

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the methods increase diffusing capacity of carbon monoxide of the patient.

The diffusing capacity of carbon monoxide, or DL_{CO} can be used in conjunction with spirometry and lung volume assessment to diagnose underlying lung disease (e.g., normal spirometry and lung volumes associated with decreased DL_{CO} may suggest anemia, pulmonary vascular disorders, early ILD, or early emphysema). In some embodiments, the patient has a diffusing capacity of carbon monoxide (DL_{CO}) less than 60%. In some embodiments, the patient has a diffusing capacity of carbon monoxide (DL_{CO}) less than 55%. In some embodiments, the patient has a diffusing capacity of carbon monoxide (DL_{CO}) less than 50%. In some embodiments, the patient has a diffusing capacity of carbon monoxide (DL_{CO}) less than 45%. In some embodiments, the patient has a diffusing capacity of carbon monoxide (DL_{CO}) less than 40%. In some embodiments, the patient has a diffusing capacity of carbon monoxide (DL_{CO}) less than 35%. In some embodiments, the patient has a diffusing capacity of carbon monoxide (DL_{CO}) less than 30%. In some embodiments, the patient has a diffusing capacity of carbon monoxide (DL_{CO}) less than 25%. In some embodiments, the patient has a diffusing capacity of carbon monoxide (DL_{CO}) less than 20%.

In some embodiments, the method increases the DL_{CO} in the patient. In some embodiments, the method increases the DL_{CO} in the patient by at least 5%. In some embodiments, the method increases the DL_{CO} in the patient by at least 10%. In some embodiments, the method increases the DL_{CO} in the patient by at least 15%. In some embodiments, the method increases the DL_{CO} in the patient by at least 20%. In some embodiments, the method increases the DL_{CO} in the patient by at least 25%. In some embodiments, the method increases the DL_{CO} in the patient by at least 30%. In some embodiments, the method increases the DL_{CO} in the patient by at least 35%. In some embodiments, the method increases the DL_{CO} in the patient by at least 40%. In some embodiments, the method increases the DL_{CO} in the patient by at least 45%. In some embodiments, the method increases the DL_{CO} in the patient by at least 50%. In some embodiments, the method increases the DL_{CO} to at least 40%. In some embodiments, the method increases the DL_{CO} to at least 45%. In some embodiments, the method increases the DL_{CO} to at least 50%. In some embodiments, the method increases the DL_{CO} to at least 55%. In some embodiments, the method increases the DL_{CO} to at least 60%. In some embodiments, the method increases the DL_{CO} to at least 65%.

### Pulmonary Fibrosis

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the methods decrease pulmonary fibrosis in the patient.

In some embodiments, the method reduces the pulmonary fibrosis in the patient by at least 10%. In some embodiments, the method reduces the pulmonary fibrosis in the patient by at least 15%. In some embodiments, the method reduces the pulmonary fibrosis in the patient by at least 20%. In some embodiments, the method reduces the pulmonary fibrosis in the patient by at least 25%. In some embodiments, the method reduces the pulmonary fibrosis in the patient by at least 30%. In some embodiments, the method reduces the pulmonary fibrosis in the patient by at least 35%. In some embodiments, the method reduces the pulmonary fibrosis in the patient by at least 40%. In some embodiments, the method reduces the pulmonary fibrosis in the patient by at least 45%. In some embodiments, the method reduces the pulmonary fibrosis in the patient by at least 50%.

### Transplant Free Survival

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the methods increase transplant free survival in the patient.

In some embodiments, the method increases transplant free survival in the patient by at least 10%. In some embodiments, the method increases transplant free survival in the patient by at least 15%. In some embodiments, the method increases transplant free survival in the patient by at least 20%. In some embodiments, the method increases transplant free survival in the patient by at least 25%. In some embodiments, the method increases transplant free survival in the patient by at least 30%. In some embodiments, the method increases transplant free survival in the patient by at least 35%. In some embodiments, the method increases transplant free survival in the patient by at least 40%. In some embodiments, the method increases transplant free survival in the patient by at least 45%. In some embodiments, the method increases transplant free survival in the patient by at least 50%.

### Death

In certain aspects, the disclosure relates to methods of treating, preventing, or reducing the progression rate and/or severity of one or more complications of pulmonary hypertension associated with lung disease, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1, wherein the methods reduce the risk of death.

In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 10%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 15%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 20%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 25%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 30%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 35%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 40%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 45%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 50%.

### Combination Therapies

Optionally, methods disclosed herein for treating, preventing, or reducing the progression rate and/or severity of pulmonary hypertension associated with lung disease (e.g., pulmonary hypertension associated with chronic obstructive pulmonary disease (COPD), interstitial lung disease (ILD), or combined pulmonary fibrosis and emphysema (CPFE)), particularly treating, preventing, or reducing the progression rate and/or severity of one or more complications of pulmonary hypertension associated with lung disease (e.g., pulmonary hypertension associated with chronic obstructive pulmonary disease (COPD), interstitial lung disease (ILD), or combined pulmonary fibrosis and emphysema (CPFE)), may further comprise administering to the patient one or more supportive therapies or additional active agents for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)). For example, the patient also may be administered one or more supportive therapies or active agents selected from the group consisting of: nitrates, hydralazine, pyridones (e.g., pirfenidone), small molecule tyrosine-kinase inhibitors (e.g., nintedanib), prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, darusentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat, vericiguat, and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SC≥015; SZC017; PEGylated derivatives of oleanolic acid; 3-O-(beta-D-glucopyranosyl) oleanolic acid; 3-O-[beta-D-glucopyranosyl-(1- ->3)-beta-D-glucopyranosyl] oleanolic acid; 3-O-[beta-D-glucopyranosyl-(1- ->2)-beta-D-glucopyranosyl] oleanolic acid; 3-O-[beta-D-glucopyranosyl-(1- ->3)-beta-D-glucopyranosyl] oleanolic acid 28-O-beta-D-glucopyranosyl ester, 3-O-[beta-D-glucopyranosyl-(1- ->2)-beta-D-glucopyranosyl] oleanolic acid 28-O-beta-D-glucopyranosyl ester; 3-O-[a-L-rhamnopyranosyl-(1- ->3)-beta-D-glucuronopyranosyl] oleanolic acid; 3-O-[alpha-L-rhamnopyranosyl-(1- ->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate), oxygen therapy, lung and/or heart transplantation. In some embodiments, the methods described herein may further comprise administering to the patient pirfenidone. In some embodiments, the methods described herein may further comprise administering to the patient nintedanib. In some embodiments, the methods described herein may further comprise administering to the patient parental prostacyclin. In some embodiments, the methods described herein may further comprise administering to the patient one additional supportive therapy or additional active agent (i.e., double therapy) for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)). In some embodiments, the methods described herein may further comprise administering to the patient two additional supportive therapies or additional active agents (i.e., triple therapy) for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)). In some embodiments, the methods described herein may further comprise administering to the patient three additional supportive therapies or additional active agents (i.e., quadruple therapy) for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)).

In some embodiments, the methods described herein may further comprise administering to the patient an angiotensin antagonist (e.g., angiotensin receptor blocker, ARB). In some embodiments, a patient is further administered one or more ARBs selected from the group consisting of losartan, irbesartan, olmesartan, candesartan, valsartan, fimasartan, azilsartan, salprisartan, and telmisartan. In some embodiments, a patient is administered losartan. In some embodiments, a patient is administered irbesartan. In some embodiments, a patient is administered olmesartan. In some embodiments, a patient is administered candesartan. In some embodiments, a patient is administered valsartan. In some embodiments, a patient is administered fimasartan. In some embodiments, a patient is administered azilsartan. In some embodiments, a patient is administered salprisartan. In some embodiments, a patient is administered telmisartan.

In some embodiments, the methods described herein may further comprise administering to the patient one or more ACE inhibitors. In some embodiments, the one or more ACE inhibitors are selected from the group consisting of benazepril, captopril, enalapril, lisinopril, perindopril, ramipril (e.g., ramipen), trandolapril, and zofenopril. In some embodiments, a patient is administered benazepril. In some embodiments, a patient is administered captopril. In some embodiments, a patient is administered enalapril. In some embodiments, a patient is administered lisinopril. In some embodiments, a patient is administered perindopril. In some embodiments, a patient is administered ramipril. In some embodiments, a patient is administered trandolapril. In some embodiments, a patient is administered zofenopril. In some embodiments, the methods described herein may further comprise administering to the patient an ARB and an ACE inhibitor. In some embodiments, an alternative approach to angiotensin antagonism is to combine an ACE inhibitor and/or ARB with an aldosterone antagonist.

In some embodiments, the one or more supportive therapies or additional active agents for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) are administered prior to administration of the ActRII polypeptide. In some embodiments, the one or more supportive therapies or additional active agents for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) are administered in combination with the ActRII polypeptide. In some embodiments, the one or more supportive therapies or additional active agents for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) are administered after the administration of the ActRII polypeptide.

### Functional Classes

Pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) at baseline can be mild, moderate or severe, as measured for example by World Health Organization (WHO) functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system: Functional Class I: pulmonary hypertension without resulting limitation of physical activity; ordinary physical activity does not cause undue dyspnea or fatigue, chest pain or near syncope; Functional Class II: pulmonary hypertension resulting in slight limitation of physical activity; patient comfortable at rest; ordinary physical activity causes undue dyspnea or fatigue, chest pain or near syncope; Functional Class III: pulmonary hypertension resulting in marked limitation of physical activity; patient comfortable at rest; less than ordinary activity causes undue dyspnea or fatigue, chest pain or near syncope; Functional Class IV: pulmonary hypertension resulting in inability to carry out any physical activity without symptoms; patient manifests signs of right-heart failure; dyspnea and/or fatigue may be present even at rest; discomfort is increased by any physical activity. In some embodiments of the methods disclosed herein, the method prevents or reduces pulmonary hypertension Functional Class progression as recognized by the World Health Organization (WHO). In some embodiments, the method prevents or reduces pulmonary hypertension Functional Class progression from Functional Class I to Class II pulmonary hypertension as recognized by the WHO. In some embodiments, the method prevents or reduces pulmonary hypertension Functional Class progression from Functional Class II to Class III pulmonary hypertension as recognized by the WHO. In some embodiments, the method prevents or reduces pulmonary hypertension Functional Class progression from Functional Class III to Class IV pulmonary hypertension as recognized by the WHO. In some embodiments, the method promotes or increases pulmonary hypertension Functional Class regression as recognized by the WHO. In some embodiments, the method promotes or increases pulmonary hypertension Functional Class regression from Class IV to Class III pulmonary hypertension as recognized by the WHO. In some embodiments, the method promotes or increases pulmonary hypertension Functional Class regression from Class III to Class II pulmonary hypertension as recognized by the WHO. In some embodiments, the method promotes or increases pulmonary hypertension Functional Class regression from Class II to Class I pulmonary hypertension as recognized by the WHO.

In some embodiments, the disclosure relates to methods of preventing or reducing pulmonary hypertension Functional Class progression comprising administering to a patient in need thereof an effective amount of an ActRII polypeptide (e.g., an amino acid sequence that is at least 90% identical to an amino acid sequence corresponding to residues 30-110 of SEQ ID NO: 1). In some embodiments, the reduction in Functional Class progression is a delay in Functional Class progression. In some embodiments, the method relates to preventing or decreasing pulmonary hypertension functional class progression as recognized by the WHO. In some embodiments, the method relates to a patient that has Functional Class I pulmonary hypertension as recognized by the WHO. In some embodiments, the method relates to preventing or reducing patient progression from Functional Class I pulmonary hypertension to Functional Class II pulmonary hypertension as recognized by the WHO. In some embodiments, the method relates to a patient that has Functional Class II pulmonary hypertension as recognized by the WHO. In some embodiments, the method relates to preventing or reducing patient progression from Functional Class II pulmonary hypertension to Functional Class Ill pulmonary hypertension as recognized by the WHO. In some embodiments, the method relates to a patient that has Functional Class III pulmonary hypertension as recognized by the WHO. In some embodiments, the method relates to preventing or reducing patient progression from Functional Class III pulmonary hypertension to Functional Class IV pulmonary hypertension as recognized by the WHO.

In certain aspects, the disclosure relates to methods of promoting or increasing pulmonary hypertension Functional Class regression in a pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1. In some embodiments, the patient has Functional Class I, Functional Class II, Functional Class III, or Functional Class IV pulmonary hypertension as recognized by the WHO. In some embodiments, the method relates to a patient that has Functional Class II pulmonary hypertension as recognized by the WHO. In some embodiments, the method relates to promoting patient regression from Functional Class II pulmonary hypertension to Functional Class I pulmonary hypertension as recognized by the WHO. In some embodiments, the method relates to a patient that has Functional Class III pulmonary hypertension as recognized by the WHO. In some embodiments, the method relates to promoting patient regression from Functional Class III pulmonary hypertension to Functional Class II pulmonary hypertension as recognized by the WHO. In some embodiments, the method relates to promoting patient regression from Functional Class III pulmonary hypertension to Functional Class I pulmonary hypertension as recognized by the WHO. In some embodiments, the method relates to a patient that has Functional Class IV pulmonary hypertension as recognized by the WHO. In some embodiments, the method relates to promoting patient regression from Functional Class IV pulmonary hypertension to Functional Class III pulmonary hypertension as recognized by the WHO. In some embodiments, the method relates to promoting patient regression from Functional Class IV pulmonary hypertension to Functional Class II pulmonary hypertension as recognized by the WHO. In some embodiments, the method relates to promoting patient regression from Functional Class IV pulmonary hypertension to Functional Class I pulmonary hypertension as recognized by the WHO.

In some embodiments, functional class regression is tested after the patient has received 4 weeks of treatment utilizing an ActRII polypeptide disclosed herein. In some embodiments, functional class regression is tested after the patient has received 8 weeks of treatment utilizing an ActRII polypeptide disclosed herein. In some embodiments, functional class regression is tested after the patient has received 12 weeks of treatment utilizing an ActRII polypeptide disclosed herein. In some embodiments, functional class regression is tested after the patient has received 16 weeks of treatment utilizing an ActRII polypeptide disclosed herein. In some embodiments, functional class regression is tested after the patient has received 20 weeks of treatment utilizing an ActRII polypeptide disclosed herein. In some embodiments, functional class regression is tested after the patient has received 22 weeks of treatment utilizing an ActRII polypeptide disclosed herein. In some embodiments, functional class regression is tested after the patient has received 24 weeks of treatment utilizing an ActRII polypeptide disclosed herein. In some embodiments, functional class regression is tested after the patient has received 26 weeks of treatment utilizing an ActRII polypeptide disclosed herein. In some embodiments, functional class regression is tested after the patient has received 28 weeks of treatment utilizing an ActRII polypeptide disclosed herein. In some embodiments, functional class regression is tested after the patient has received 48 weeks of treatment utilizing an ActRII polypeptide disclosed herein.

### Sustained Therapeutic Effect

In certain aspects, the disclosure relates to methods of treating, preventing, or reducing the progression rate and/or severity of one or more complications of pulmonary hypertension associated with lung disease in a sustained manner, comprising administering to a patient in need thereof an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113,114, 115, 116, 117, 118, 119, 120, 121, 122, 123, 124, 125, 126, 127, 128, 129, 130, 131, 132, 133, 134, or 135 of SEQ ID NO: 1. In some embodiments, the sustained manner comprises a persistent therapeutic effect following the reduction in administration of an ActRII polypeptide described herein. In some embodiments, the sustained manner comprises a persistent therapeutic effect following the withdrawal of administration of an ActRII polypeptide described herein. In some embodiments, the persistent therapeutic effect relates to maintaining functional or hematologic measurements over time. In some embodiments, the persistent therapeutic effect is measured as a sustained reduction in PVR. In some embodiments, the patient's PVR level does not increase for at least 1 week to at least 12 weeks following withdrawal of an ActRII polypeptide treatment described herein. In some embodiments, the patient's PVR level does not increase for at least 1 week following withdrawal of an ActRII polypeptide treatment described herein. In some embodiments, the patient's PVR level does not increase for at least 2 weeks following withdrawal of an ActRII polypeptide treatment described herein. In some embodiments, the patient's PVR level does not increase for at least 3 weeks following withdrawal of an ActRII polypeptide treatment described herein. In some embodiments, the patient's PVR level does not increase for at least 4 weeks following withdrawal of an ActRII polypeptide treatment described herein. In some embodiments, the patient's PVR level does not increase for at least 5 weeks following withdrawal of an ActRII polypeptide treatment described herein. In some embodiments, the patient's PVR level does not increase for at least 6 weeks following withdrawal of an ActRII polypeptide treatment described herein. In some embodiments, the patient's PVR level does not increase for at least 1 month to at least 6 months following withdrawal of an ActRII polypeptide treatment described herein.

In certain aspects, the disclosure relates to methods of treating or preventing cardiopulmonary remodeling associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) in a patient, comprising administering to a patient in need thereof an effective amount of an ActRIIA polypeptide, wherein said method slows down cardiac remodeling and/or reverses cardiac remodeling. In some embodiments, the reversal is a sustained reversal. In some embodiments, the cardiac remodeling is ventricle remodeling. In some embodiments, the ventricle remodeling is left ventricular remodeling. In some embodiments, the ventricle remodeling is right ventricular remodeling. In some embodiments, the cardiac remodeling is ventricular dilation. In some embodiments, the method decreases interventricular septal end diastole. In some embodiments, the method decreases posterior wall end diastole.

In some embodiments, echocardiographic measurements may be used to assess the persistent therapeutic effect. In some embodiments, the echocardiographic measurements include, but are not limited to, RV fractional area change (RVFAC), sPAP, tricuspid annular systolic velocity (TASV), and Tei index. In some embodiments, a patient treated with an ActRIIA polypeptide disclosed herein shows a persistent therapeutic effect. In some embodiments, the persistent therapeutic effect results in decreased intrusion of the ventral wall into the left ventricle. In some embodiments, the persistent therapeutic effect results in an increase in right ventricular fractional area change (RVFAC).

### Measuring Various Parameters Over Time

In certain embodiments, one or more of the measurements of pulmonary hypertension (e.g., pulmonary hypertension associated with lung disease (e.g., pulmonary hypertension associated with chronic obstructive pulmonary disease (COPD), interstitial lung disease (ILD), or combined pulmonary fibrosis and emphysema (CPFE))) described herein can be measured over various periods of treatment time. In some embodiments, one or more of the measurements of pulmonary hypertension described herein is measured after the patient has received 4 weeks of treatment utilizing an ActRII polypeptide disclosed herein. In some embodiments, one or more of the measurements of pulmonary hypertension described herein is measured after the patient has received 8 weeks of treatment utilizing an ActRII polypeptide disclosed herein. In some embodiments, one or more of the measurements of pulmonary hypertension described herein is measured after the patient has received 12 weeks of treatment utilizing an ActRII polypeptide disclosed herein. In some embodiments, one or more of the measurements of pulmonary hypertension described herein is measured after the patient has received 16 weeks of treatment utilizing an ActRII polypeptide disclosed herein. In some embodiments, one or more of the measurements of pulmonary hypertension described herein is measured after the patient has received 20 weeks of treatment utilizing an ActRII polypeptide disclosed herein. In some embodiments, one or more of the measurements of pulmonary hypertension described herein is measured after the patient has received 22 weeks of treatment utilizing an ActRII polypeptide disclosed herein. In some embodiments, one or more of the measurements of pulmonary hypertension described herein is measured after the patient has received 24 weeks of treatment utilizing an ActRII polypeptide disclosed herein. In some embodiments, one or more of the measurements of pulmonary hypertension described herein is measured after the patient has received 26 weeks of treatment utilizing an ActRII polypeptide disclosed herein. In some embodiments, one or more of the measurements of pulmonary hypertension described herein is measured after the patient has received 28 weeks of treatment utilizing an ActRII polypeptide disclosed herein. In some embodiments, one or more of the measurements of pulmonary hypertension described herein is measured after the patient has received 48 weeks of treatment utilizing an ActRII polypeptide disclosed herein.

### 5. Pharmaceutical Compositions & Modes of Administration

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than the ActRII polypeptides which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more ActRII polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind described herein.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Further, the composition may be encapsulated or injected in a form for delivery to a target tissue site. In certain embodiments, compositions of the present invention may include a matrix capable of delivering one or more therapeutic compounds (e.g., ActRII polypeptides) to a target tissue site, providing a structure for the developing tissue and optimally capable of being resorbed into the body. For example, the matrix may provide slow release of the ActRII polypeptide. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments, methods of the invention can be administered for orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., ActRII polypeptides). The various factors include, but are not limited to, the patient's age, sex, and diet, the severity disease, time of administration, and other clinical factors. Optionally, the dosage may vary with the type of matrix used in the reconstitution and the types of compounds in the composition. In some embodiments, a patient's hematologic parameters can be monitored by periodic assessments in order to determine if they have higher than normal red blood cell levels and/or hemoglobin levels (e.g., hemoglobin levels >16.0 g/dL or hemoglobin levels >18.0 g/dL). In some embodiments, patient's having higher than normal red blood cell levels and/or hemoglobin levels may receive a delayed or reduced dose until the levels have returned to a normal or acceptable level.

The probability of a patient having hemoglobin levels greater than 18 g/dL or increases in hemoglobin of greater than 2 g/dL may be higher during initial treatment with an ActRII polypeptide. In certain embodiments, a dosing regimen can be used to prevent, ameliorate, or decrease the adverse changes in hemoglobin levels. In some embodiments, ActRII polypeptides of the disclosure are administered using a dosing regimen. In some embodiments, the method comprises administering a dosing regimen of a therapeutically effective amount of an ActRII polypeptide as disclosed herein to a patient, comprising a first dose of between 0.1 mg/kg and 1.0 mg/kg of said polypeptide for a first period of time, and a second dose of between 0.1 mg/kg and 1.0 mg/kg of said polypeptide subsequently administered for a second period of time. In some embodiments, the method comprises administering a dosing regimen of therapeutically effective amount of an ActRII polypeptide as disclosed herein to a patient, comprising a first dose of between 0.1 mg/kg and 1.0 mg/kg of said polypeptide for a first period of time, a second dose of between 0.1 mg/kg and 1.0 mg/kg of said polypeptide administered for a second period of time, and a third dose of between 0.1 mg/kg and 1.0 mg/kg of said polypeptide subsequently administered for a third period of time. In some embodiments, the first dose of ActRII polypeptide is administered to a patient in an amount from about 0.2 mg/kg to about 0.4 mg/kg. In some embodiments, the first dose of ActRII polypeptide is administered to a patient at a dose of 0.3 mg/kg. In some embodiments, the second dose of ActRII polypeptide is administered to a patient in an amount from about 0.5 mg/kg to about 0.8 mg/kg. In some embodiments, the second dose of ActRII polypeptide is administered to a patient at a dose of 0.7 mg/kg. In some embodiments, the third dose of ActRII polypeptide is administered to a patient in an amount from about 0.2 mg/kg to about 0.4 mg/kg. In some embodiments, the third dose of ActRII polypeptide is administered to a patient at a dose of 0.3 mg/kg.

In some embodiments, the dosing regimen comprises administering a first dose of ActRII polypeptide to a patient in an amount of 0.3 mg/kg followed by administration of a second dose of ActRII polypeptide to the patient in an amount of 0.7 mg/kg. In some embodiments, the dosing regimen comprises administering a first dose of ActRII polypeptide to a patient in an amount of 0.3 mg/kg, administering a second dose of ActRII polypeptide to the patient in an amount of 0.7 mg/kg, and administering a third dose of ActRII polypeptide to the patient in an amount of 0.3 mg/kg. In some embodiments, the second dose exceeds the first dose. In some embodiments, the first dose exceeds the second dose. In some embodiments, the third dose exceeds the second dose. In some embodiments, the second dose exceeds the third dose. In some embodiments, the first period of time is at least 3 weeks. In some embodiments, the second period of time is at least 3 weeks. In some embodiments, the third period of time is at least 3 weeks. In some embodiments, the second period of time is at least 21 weeks. In some embodiments, the second period of time is at least 45 weeks. In some embodiments, the second period of time exceeds the first period of time. In some embodiments, the third period of time exceeds the first period of time. In some embodiments, the third period of time exceeds the second period of time.

In some embodiments, the change in dosing between the first dose and the second dose is determined by the attending physician considering various factors (e.g., hemoglobin levels).

In some embodiments, the change in dosing between the second dose and the third dose is determined by the attending physician considering various factors (e.g., hemoglobin levels).

In some embodiments, the various factors include, but are not limited to, the patient's change in hematologic parameters over a period of time. In some embodiments, a patient's hematologic parameters are monitored in order to determine if they have higher than normal red blood cell levels and/or hemoglobin levels (e.g., hemoglobin levels >16.0 g/dL or hemoglobin levels >18.0 g/dL). In some embodiments, a patient's hematologic parameters are monitored in order to determine if they have a higher than normal increase in hemoglobin levels over a period of time (e.g., hemoglobin level increase of >2 g/dL in less than 3 weeks). In some embodiments, the patient's dose of an ActRII polypeptide as disclosed herein will be decreased (e.g., decrease in dose from 0.7 mg/kg to 0.3 mg/kg) if one or more of the patient's hematologic parameters before or during treatment is abnormal. In some embodiments, the patient's dose of an ActRII polypeptide as disclosed herein will be maintained (e.g., maintained at 0.3 mg/kg or 0.7 mg/kg) if one or more of the patient's hematologic parameters before or during treatment is abnormal.

In some embodiments, the dosing regimen prevents, ameliorates, or decreases adverse effects of the ActRII polypeptide. In some embodiments, administration of an ActRII polypeptide in accordance with the dosage regimen as provided herein results in decreased adverse side effects. In some embodiments, administration of an ActRII polypeptide in accordance with the dosage regimen as provided herein decreases the probability of having hemoglobin levels greater than 18 g/dL during the first period of time. In some embodiments, administration of an ActRII polypeptide in accordance with the dosage regimen as provided herein decreases the probability of having hemoglobin levels greater than 18 g/dL in the first 3 weeks of treatment. In some embodiments, administration of an ActRII polypeptide in accordance with the dosage regimen as provided herein decreases the probability of increasing hemoglobin levels by greater than 2 g/dL during the first period of time. In some embodiments, administration of an ActRII polypeptide in accordance with the dosage regimen as provided herein decreases the probability of increasing hemoglobin levels by greater than 2 g/dL in the first 3 weeks of treatment.

In some embodiments, ActRII polypeptides of the disclosure are administered at a dosing range of 0.1 mg/kg to 2.0 mg/kg. In some embodiments, ActRII polypeptides of the disclosure are administered at 0.1 mg/kg. In some embodiments, ActRII polypeptides of the disclosure are administered at 0.2 mg/kg. In some embodiments, ActRII polypeptides of the disclosure are administered at 0.3 mg/kg. In some embodiments, ActRII polypeptides of the disclosure are administered at 0.4 mg/kg. In some embodiments, ActRII polypeptides of the disclosure are administered at 0.5 mg/kg. In some embodiments, ActRII polypeptides of the disclosure are administered at 0.6 mg/kg. In some embodiments, ActRII polypeptides of the disclosure are administered at 0.7 mg/kg. In some embodiments, ActRII polypeptides of the disclosure are administered at 0.8 mg/kg. In some embodiments, ActRII polypeptides of the disclosure are administered at 0.9 mg/kg. In some embodiments, ActRII polypeptides of the disclosure are administered at 1.0 mg/kg. In some embodiments, ActRII polypeptides of the disclosure are administered at 1.1 mg/kg. In some embodiments, ActRII polypeptides of the disclosure are administered at 1.2 mg/kg. In some embodiments, ActRII polypeptides of the disclosure are administered at 1.3 mg/kg. In some embodiments, ActRII polypeptides of the disclosure are administered at 1.4 mg/kg. In some embodiments, ActRII polypeptides of the disclosure are administered at 1.5 mg/kg. In some embodiments, ActRII polypeptides of the disclosure are administered at 1.6 mg/kg. In some embodiments, ActRII polypeptides of the disclosure are administered at 1.7 mg/kg. In some embodiments, ActRII polypeptides of the disclosure are administered at 1.8 mg/kg. In some embodiments, ActRII polypeptides of the disclosure are administered at 1.9 mg/kg. In some embodiments, ActRII polypeptides of the disclosure are administered at 2.0 mg/kg.

In certain embodiments, ActRII polypeptides of the disclosure are administered once a day. In certain embodiments, ActRII polypeptides of the disclosure are administered twice a day. In certain embodiments, ActRII polypeptides of the disclosure are administered once a week. In certain embodiments, ActRII polypeptides of the disclosure are administered twice a week. In certain embodiments, ActRII polypeptides of the disclosure are administered three times a week. In certain embodiments, ActRII polypeptides of the disclosure are administered every two weeks. In certain embodiments, ActRII polypeptides of the disclosure are administered every three weeks. In certain embodiments, ActRII polypeptides of the disclosure are administered every four weeks. In certain embodiments, ActRII polypeptides of the disclosure are administered every month.

In certain embodiments, the present invention also provides gene therapy for the in vivo production of ActRII polypeptides. Such therapy would achieve its therapeutic effect by introduction of the ActRII polypeptide polynucleotide sequences into cells or tissues having the disorders as listed above. Delivery of ActRII polypeptide polynucleotide sequences can be achieved using a recombinant expression vector such as a chimeric virus or a colloidal dispersion system. Targeted liposomes can be used for therapeutic delivery of ActRII polypeptide polynucleotide sequences.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Targeting may be accomplished by using an antibody. Those of skill in the art will recognize that specific polynucleotide sequences can be inserted into the retroviral genome or attached to a viral envelope to allow target specific delivery of the retroviral vector containing the ActRII polypeptide. In one embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for ActRII polypeptide polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. One colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### 6. Kits

The disclosure provides a kit comprising a lyophilized polypeptide and an injection device. In certain embodiments, the lyophilized polypeptide comprises an ActRII polypeptide (e.g., a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1), or fragments, functional variants, or modified forms thereof. In certain embodiments, the lyophilized polypeptide binds to one or more ligands selected from the group consisting of activin A, activin B, and GDF11. In certain such embodiments, the lyophilized polypeptide further binds to one or more ligands selected from the group consisting of BMP10, GDF8, and BMP6. In certain embodiments, the lyophilized polypeptide binds to activin and/or GDF11.

In some embodiments, the lyophilized polypeptide comprises a polypeptide that comprises, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of a polypeptide beginning at a residue corresponding to any one of amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113,114, 115, 116, 117, 118, 119, 120, 121, 122, 123, 124, 125, 126, 127, 128, 129, 130, 131, 132, 133, 134, or 135) of SEQ ID NO: 1. In certain such embodiments, the polypeptide comprises an amino acid sequence that is least 90%, 95%, or 99% identical to an amino acid sequence corresponding to residues 30-110 of SEQ ID NO: 1, wherein the polypeptide binds to activin and/or GDF11. In certain embodiments, the polypeptide comprises the amino acid sequence corresponding to residues 30-110 of SEQ ID NO: 1. In other embodiments, the polypeptide consists of the amino acid sequence corresponding to residues 30-110 of SEQ ID NO: 1. In certain embodiments, the polypeptide is a polypeptide comprising an amino acid sequence that is at least 90%, 95%, or 99% identical to the amino acid sequence corresponding to residues 21-135 of SEQ ID NO: 1. In certain embodiments, the polypeptide comprises the amino acid sequence corresponding to residues 21-135 of SEQ ID NO: 1. In other embodiments, the polypeptide consists of the amino acid sequence corresponding to residues 21-135 of SEQ ID NO: 1.

In some embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 2. In certain embodiments, the polypeptide consists essentially of the amino acid sequence of SEQ ID NO: 2. In other embodiments, the polypeptide consists of the amino acid sequence of SEQ ID NO: 2.

In some embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3. In certain embodiments, the polypeptide consists of the amino acid sequence of SEQ ID NO: 3. In other embodiments, the polypeptide consists of the amino acid sequence of SEQ ID NO: 3.

In certain embodiments of the foregoing, the lyophilized polypeptide comprises a fusion protein further comprising an Fc domain of an immunoglobulin. In certain such embodiments, the Fc domain of the immunoglobulin is an Fc domain of an IgG1 immunoglobulin. In other embodiments, the fusion protein further comprises a linker domain positioned between the polypeptide domain and the Fc domain of the immunoglobulin. In certain embodiments, the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21). In certain embodiments, the linker domain comprises TGGG (SEQ ID NO: 20).

In certain embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 23. In certain embodiments, the polypeptide consists of the amino acid sequence of SEQ ID NO: 23. In other embodiments, the polypeptide consists of the amino acid sequence of SEQ ID NO: 23.

In certain embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 30. In certain embodiments, the polypeptide consists of the amino acid sequence of SEQ ID NO: 30. In other embodiments, the polypeptide consists of the amino acid sequence of SEQ ID NO: 30.

In certain embodiments, the lyophilized polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 41. In certain embodiments, the polypeptide consists of the amino acid sequence of SEQ ID NO: 41. In other embodiments, the polypeptide consists of the amino acid sequence of SEQ ID NO: 41.

In certain embodiments, the lyophilized polypeptide is part of a homodimer protein complex. In certain embodiments, the polypeptide is glycosylated.

The disclosure provides a kit comprising a sterile powder comprising a lyophilized polypeptide as disclosed herein and an injection device. In some embodiments of the kits disclosed herein, the sterile powder comprising a lyophilized polypeptide is pre-filled in one or more containers, such as one or more vials.

In certain embodiments, the pH range for the sterile powder comprising a lyophilized polypeptide is from 7 to 8. In some embodiments, the sterile powder comprising a lyophilized polypeptide further comprises a buffering agent. In some embodiments, the buffering agent may be added in an amount of at least 10 mM. In some embodiments, the buffering agent may be added in an amount in the range of between about 10 mM to about 200 mM. In some embodiments, the buffering agent comprises citric acid monohydrate and/or trisodium citrate dehydrate.

In some embodiments, the sterile powder comprising a lyophilized polypeptide further comprises a surfactant. In some embodiments, the surfactant comprises a polysorbate. In some embodiments, the surfactant comprises polysorbate 80.

In some embodiments, the sterile powder comprising a lyophilized polypeptide further comprises a lyoprotectant. In some embodiments, the lyoprotectant comprises a sugar, such as disaccharides (e.g., sucrose). In some embodiments, the lyoprotectant comprises sucrose, trehalose, mannitol, polyvinylpyrrolidone (PVP), dextrose, and/or glycine. In some embodiments, the lyoprotectant comprises sucrose. In some embodiments, the sterile powder comprises the lyoprotectant and lyophilized polypeptide in a weight ratio of at least 1:1 lyophilized polypeptide to lyoprotectant. In some embodiments, the sterile powder comprises the lyoprotectant and lyophilized polypeptide in a weight ratio of from 1:1 to 1:10 lyophilized polypeptide to lyoprotectant. In some embodiments, the sterile powder comprises the lyoprotectant and lyophilized polypeptide in a weight ratio of 1:1, 1:2, 1:3, 1:4, 1:5, 1:6, 1:7, 1:8, 1:9, or 1:10 lyophilized polypeptide to lyoprotectant. In some embodiments, the sterile powder comprises the lyoprotectant and lyophilized polypeptide in a weight ratio of 1:6 lyophilized polypeptide to lyoprotectant. In certain embodiments of the foregoing, the sterile powder comprises lyoprotectant in an amount sufficient to stabilize the lyophilized polypeptide.

In certain embodiments of the kits disclosed herein, the injection device comprises a syringe. In certain such embodiments, the syringe is pre-filled with a reconstitution solution. In some embodiments, the reconstitution solution comprises a pharmaceutically acceptable carrier and/or excipient. In some embodiments, the pharmaceutically acceptable carrier comprises aqueous solutions such as water, physiologically buffered saline, or other solvents or vehicles such as glycols, glycerol, oils or injectable organic esters. In some embodiments, the pharmaceutically acceptable excipient comprises a pharmaceutically acceptable excipient selected from calcium phosphates, calcium carbonates, calcium sulfates, halites, metallic oxides, sugars, sugar alcohols, starch, glycols, povidones, mineral hydrocarbons, acrylic polymers, fatty alcohols, mineral stearates, glycerin, and/or lipids. In certain embodiments, the reconstitution solution comprises pharmaceutically acceptable sterile isotonic aqueous or nonaqueous solutions, dispersions, suspensions or emulsions. In certain such embodiments, the reconstitution solution comprises antioxidants, buffers, bacteriostats, and/or solutes which render the formulation isotonic with the blood of the intended recipient. In other embodiments, the reconstitution solution comprises suspending or thickening agents.

In certain embodiments of the kits disclosed herein, the kit further comprises a vial adapter. In some embodiments, the vial pre-filled with sterile powder comprising a lyophilized polypeptide attaches to one end of the vial adapter. In some embodiments, the syringe pre-filled with a reconstitution solution as disclosed herein attaches to an end of the vial adapter. In some embodiments, the syringe pre-filled with a reconstitution solution as disclosed herein and the vial pre-filled with sterile powder comprising a lyophilized polypeptide are attached to opposite ends of the vial adapter. In some embodiments, the reconstitution solution is transferred from the pre-filled syringe to the vial. In some embodiments, transfer of the reconstitution solution to the vial pre-filled with sterile powder comprising a lyophilized polypeptide reconstitutes the lyophilized polypeptide into a sterile injectable solution. In some embodiments, the lyophilized polypeptide is reconstituted into a sterile injectable solution. In some embodiments, the lyophilized polypeptide is reconstituted into a sterile injectable solution prior to use.

In other embodiments of the kits disclosed herein, the kit further comprises a pump apparatus. In certain embodiments, the pump apparatus comprises an electromechanical pumping assembly. In certain embodiments, the pump apparatus comprises a reservoir for holding a sterile injectable solution. In certain embodiments, the reservoir holds 1 mL of sterile injectable solution. In certain embodiments, the pump apparatus comprises one or more vials or cartridges comprising a sterile injectable solution. In certain embodiments, the vials or cartridges are prefilled with sterile injectable solution. In certain embodiments, the vials or cartridges comprise sterile injectable solution reconstituted from a lyophilized polypeptide. In certain embodiments, the reservoir is coupled to the vial or cartridge. In certain embodiments, the vial or cartridge holds 1-20 mL of sterile injectable solution. In certain embodiments, the electromechanical pumping assembly comprises a pump chamber. In certain embodiments, the electromechanical pumping assembly is coupled to the reservoir. In certain embodiments, the sterile injectable solution is received from the reservoir into the pump chamber. In some embodiments, the electromechanical pumping assembly comprises a plunger that is disposed such that sterile injectable solution in the pump chamber is in direct contact with the plunger. In certain embodiments, a sterile injectable solution is received from the reservoir into the pump chamber during a first pumping phase, and is delivered from the pump chamber to a subject during a second pumping phase. In certain embodiments, the electromechanical pumping assembly comprises control circuitry. In certain embodiments, control circuitry drives the plunger to (a) draw the sterile injectable solution into the pump chamber during the first pumping phase and (b) deliver the sterile injectable solution from the pump chamber in a plurality of discrete motions of the plunger during the second pumping phase, thereby delivering the therapeutic substance to the subject in a plurality of controlled and discrete dosages throughout the second pumping phase. In certain embodiments, a cycle of alternating the first and second pumping phases may be repeated until a desired dose is administered. In certain embodiments, the pump apparatus is coupled to a wearable patch. In certain embodiments, the pump apparatus is a wearable pump apparatus. In some embodiments, the pump apparatus administers a dose every 3 weeks. In some embodiments, the pump apparatus administers the dose via subcutaneous injection

The disclosure provides a kit used for reconstituting a lyophilized polypeptide into a sterile injectable solution. In certain embodiments, the resulting sterile injectable solution is useful in the methods disclosed herein.

In certain embodiments of the kits disclosed herein, the kit further comprises an injectable device for use in administering the sterile injectable solution parenterally. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose. In some embodiments, the therapeutically effective dose comprises a weight based dose. In some embodiments, the weight based dose is 0.3 mg/kg. In some embodiments, the weight based dose is 0.7 mg/kg.

In some embodiments of the kits disclosed herein, the kit further comprises one or more vials or cartridges containing the lyophilized polypeptide. In some embodiments, the kit comprises at least two vials or cartridges containing the lyophilized polypeptide. In some embodiments, the kit comprises at least three vials or cartridges containing the lyophilized polypeptide. In some embodiments, the two vials can contain the same or different amounts of the lyophilized polypeptide. In some embodiments, the vials or cartridges comprise a vial or cartridge containing between 25 mg to 60 mg of lyophilized polypeptide. In some embodiments, at least one of the vials or cartridges comprise a vial or cartridge containing 60 mg of lyophilized polypeptide. In some embodiments, at least one of the vials or cartridges comprise a vial or cartridge containing 45 mg of lyophilized polypeptide. In some embodiments, at least one of the vials or cartridges comprise a vial or cartridge containing 30 mg of lyophilized polypeptide. In some embodiments, at least one of the vials or cartridges comprise a vial or cartridge containing 25 mg of lyophilized polypeptide. In some embodiments, a first vial or cartridge contains 45 mg of lyophilized polypeptide and a second vial or cartridge contains 60 mg of lyophilized polypeptide. In some embodiments, a first vial or cartridge contains 30 mg of lyophilized polypeptide and a second vial or cartridge contains 60 mg of lyophilized polypeptide. In some embodiments, a first vial or cartridge contains 30 mg of lyophilized polypeptide, a second vial or cartridge contains 45 mg of lyophilized polypeptide, and a third vial or cartridge contains 60 mg of lyophilized polypeptide. In some embodiments, a first vial or cartridge contains 25 mg of lyophilized polypeptide, a second vial or cartridge contains 45 mg of lyophilized polypeptide, and a third vial or cartridge contains 60 mg of lyophilized polypeptide. In some embodiments, the one or more vials or cartridges are refrigerated at 2-8° C.

### 7. Exemplification

The disclosure above will be more readily understood by reference to the following examples, which are included merely for purposes of illustration of certain embodiments of the invention, and are not intended to limiting.

### Example 1: ActRIIA-Fc Fusion Proteins

A soluble ActRIIA fusion protein was constructed that has the extracellular domain of human ActRIIA fused to a human or mouse Fc domain with a minimal linker in between. The constructs are referred to as ActRIIA-hFc and ActRIIA-mFc, respectively.

ActRIIA-hFc is shown below as purified from CHO cell lines (SEQ ID NO: 23):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K

An additional ActRIIA-hFc lacking the C-terminal lysine is shown below as purified from CHO cell lines (SEQ ID NO: 41):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG

The ActRIIA-hFc and ActRIIA-mFc proteins were expressed in CHO cell lines. Three different leader sequences were considered:
(i) Honey bee melittin (HBML): MKFLVNVALVFMVVYISYIYA (SEQ ID NO: 24)
(ii) Tissue plasminogen activator (TPA): MDAMKRGLCCVLLLCGAVFVSP (SEQ ID NO: 25)
(iii) Native: MGAAAKLAFAVFLISCSSGA (SEQ ID NO: 26).

The selected form employs the TPA leader and has the following unprocessed amino acid sequence:

(SEQ ID NO: 27)
MDAMKRGLCCVLLLCGAVFVSPGAAILGRSETQECLFFNANWEKDRTNQ
TGVEPCYGDKDKRRHCFATWKNISGSIEIVKQGCWLDDINCYDRTDCVE
KKDSPEVYFCCCEGNMCNEKFSYFPEMEVTQPTSNPVTPKPPTGGGTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPVPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK

This polypeptide is encoded by the following nucleic acid sequence:

(SEQ ID NO: 28)
ATGGATGCAATGAAGAGAGGGCTCTGCTGTGTGCTGCTGCTGTGTGGAG
CAGTCTTCGTTTCGCCCGGCGCCGCTATACTTGGTAGATCAGAAACTCA
GGAGTGTCTTTTTTTAATGCTAATTGGGAAAAAGACAGAACCAATCAAA
CTGGTGTTGAACCGTGTTATGGTGACAAAGATAAACGGCGGCATTGTTT
TGCTACCTGGAAGAATATTTCTGGTTCCATTGAATAGTGAAACAAGGTT
GTTGGCTGGATGATATCAACTGCTATGACAGGACTGATTGTGTAGAAAA
AAAAGACAGCCCTGAAGTATATTTCTGTTGCTGTGAGGGCAATATGTGT
AATGAAAAGTTTTCTTATTTTCCGGAGATGGAAGTCACACAGCCCACTT
CAAATCCAGTTACACCTAAGCCACCCACCGGTGGTGGAACTCACACATG
CCCACCGTGCCCAGCACCTGAACTCCTGGGGGGACCGTCAGTCTTCCTC
TTCCCCCCAAAACCCAAGGACACCCTCATGATCTCCCGGACCCCTGAGG
TCACATGCGTGGTGGTGGACGTGAGCCACGAAGACCCTGAGGTCAAGTT
CAACTGGTACGTGGACGGCGTGGAGGTGCATAATGCCAAGACAAAGCCG
CGGGAGGAGCAGTACAACAGCACGTACCGTGTGGTCAGCGTCCTCACCG
TCCTGCACCAGGACTGGCTGAATGGCAAGGAGTACAAGTGCAAGGTCTC
CAACAAAGCCCTCCCAGTCCCCATCGAGAAAACCATCTCCAAAGCCAAA
GGGCAGCCCCGAGAACCACAGGTGTACACCCTGCCCCCATCCCGGGAGG
AGATGACCAAGAACCAGGTCAGCCTGACCTGCCTGGTCAAAGGCTTCTA
TCCCAGCGACATCGCCGTGGAGTGGGAGAGCAATGGGCAGCCGGAGAAC
AACTACAAGACCACGCCTCCCGTGCTGGACTCCGACGGCTCCTTCTTCC
TCTATAGCAAGCTCACCGTGGACAAGAGCAGGTGGCAGCAGGGGAACGT
CTTCTCATGCTCCGTGATGCATGAGGCTCTGCACAACCACTACACGCAG
AAGAGCCTCTCCCTGTCTCCGGGTAAATGAGAATTC

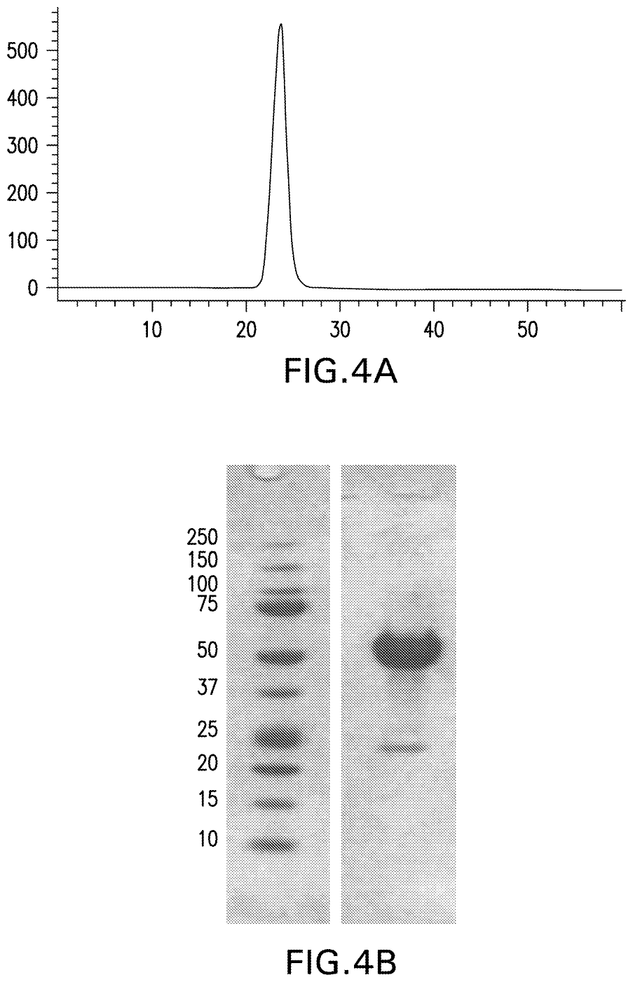

Both ActRIIA-hFc and ActRIIA-mFc were remarkably amenable to recombinant expression. As shown in FIGS. 4A and 4B, the protein was purified as a single, well-defined peak of protein. N-terminal sequencing revealed a single sequence of -ILGRSETQE (SEQ ID NO: 29). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIA-hFc protein was purified to a purity of >98% as determined by size exclusion chromatography and >95% as determined by SDS PAGE.

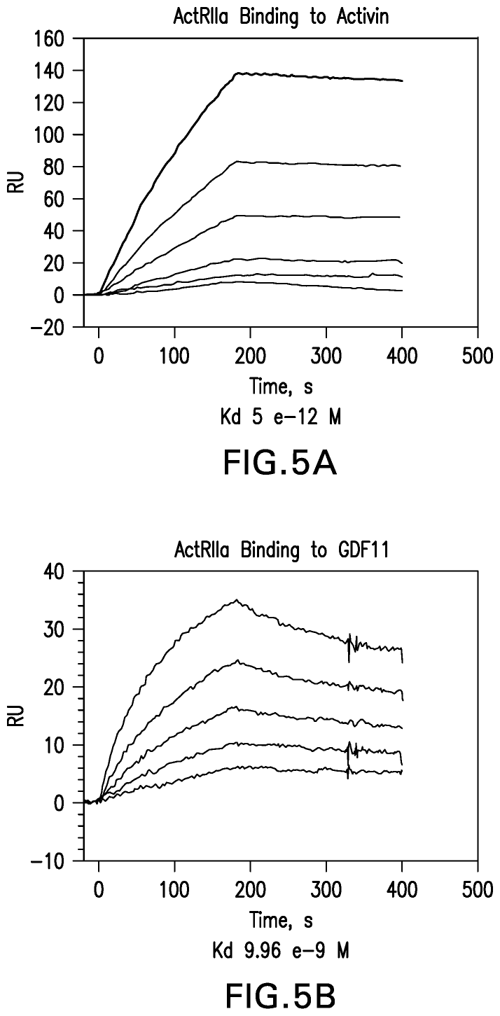

ActRIIA-hFc and ActRIIA-mFc showed a high affinity for ligands. GDF11 or activin A were immobilized on a Biacore™ CM5 chip using standard amine-coupling procedure. ActRIIA-hFc and ActRIIA-mFc proteins were loaded onto the system, and binding was measured. ActRIIA-hFc bound to activin with a dissociation constant (K_{D}) of 5×10¹² and bound to GDF11 with a K_{D} of 9.96×10⁹. See FIGS. 5A and 5B. Using a similar binding assay, ActRIIA-hFc was determined to have high to moderate affinity for other TGF-beta superfamily ligands including, for example, activin B, GDF8, BMP6, and BMP10. ActRIIA-mFc behaved similarly.

The ActRIIA-hFc was very stable in pharmacokinetic studies. Rats were dosed with 1 mg/kg, 3 mg/kg, or 10 mg/kg of ActRIIA-hFc protein, and plasma levels of the protein were measured at 24, 48, 72, 144 and 168 hours. In a separate study, rats were dosed at 1 mg/kg, 10 mg/kg, or 30 mg/kg. In rats, ActRIIA-hFc had an 11-14 day serum half-life, and circulating levels of the drug were quite high after two weeks (11 μg/ml, 110 μg/ml, or 304 μg/ml for initial administrations of 1 mg/kg, 10 mg/kg, or 30 mg/kg, respectively.) In cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 2: Characterization of an ActRIIA-hFc Protein

ActRIIA-hFc fusion protein was expressed in stably transfected CHO-DUKX B11 cells from a pAID4 vector (SV40 ori/enhancer, CMV promoter), using a tissue plasminogen leader sequence of SEQ ID NO: 25. The protein, purified as described above in Example 1, had a sequence of SEQ ID NO: 23. The Fc portion is a human IgG1 Fc sequence, as shown in SEQ ID NO: 23. Protein analysis reveals that the ActRIIA-hFc fusion protein is formed as a homodimer with disulfide bonding.

The CHO-cell-expressed material has a higher affinity for activin B ligand than that reported for an ActRIIA-hFc fusion protein expressed in human 293 cells [see, del Re et al. (2004) J Biol Chem. 279(51):53126-53135]. Additionally, the use of the TPA leader sequence provided greater production than other leader sequences and, unlike ActRIIA-Fc expressed with a native leader, provided a highly pure N-terminal sequence. Use of the native leader sequence resulted in two major species of ActRIIA-Fc, each having a different N-terminal sequence.

### Example 3: Alternative ActRIIA-Fc Proteins

A variety of ActRIIA variants that may be used according to the methods described herein are described in the International Patent Application published as WO2006/012627 (see e.g., pp. 55-58), incorporated herein by reference in its entirety. An alternative construct may have a deletion of the C-terminal tail (the final 15 amino acids of the extracellular domain of ActRIIA. The sequence for such a construct is presented below (Fc portion underlined) (SEQ

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMTGGGTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPVPIEKTISKAKGQPREPQVYTLPPSREEMTKNQV
SLTCLVKGFYPSDIAVEWESNGQPENNYKTTPPVLDSDGSFFLYSKLTV
DKSRWQQGNVFSCSVMHEALHNHYTQKSLSLSPGK

### Example 4: Effects of an ActRIIA-mFc on Group 3 Pulmonary Hypertension in Two Bleomycin-Induced Pulmonary Hypertension and Fibrosis Rat Model

The effects of an ActRIIA-mFc fusion protein (ActRIIA-mFc homodimer as described in Example 1) was examined in two rat models of Group 3 pulmonary hypertension (Grp3-PH) [Xiong et al., Hypertension 71(1):34-55 (2018); Schroll et al., Respir Physiol Neurobiol 170(1):32-36 (2010)].

In one model, twelve Wistar male rats were intratracheally administered with a single dose of bleomycin (Bleo, 0.6U/rat) at day0 and randomized into two treatment groups (6 rats per group): 1) treatment with monocrotaline (MCT, 60 mg/kg administered s.c. as a single dose at day 7 of study) and Tris buffered saline (s.c. as 1 ml/kg every three days) (vehicle treatment group), 2) treatment with MCT (60 mg/kg administered s.c. as a single dose at day 7 of study) and ActRIIA-mFc (5 mg/kg administered s.c. every three days). Rats were treated for 35 days. Body weights were recorded weekly throughout the study.

On day 42, rats were anesthetized with ˜3-4% isoflurane and placed on controlled heating pads. Right ventricular systolic pressure (RVSP) were measured by advancing a 2F curve tip pressure transducer catheter, (SPR-513, Millar Instruments) into the right ventricle (RV) via right jugular vein under ˜1.5-2% isofluorane anesthesia. RV hypertrophy was assessed by taking the weight ratio of RV free wall and LV+Septum (RV/LV+S, Fulton's Index). Lungs were collected, fixed in 10% formalin, embedded in paraffin, and sectioned for Masson's trichrome stain to assess fibrosis.

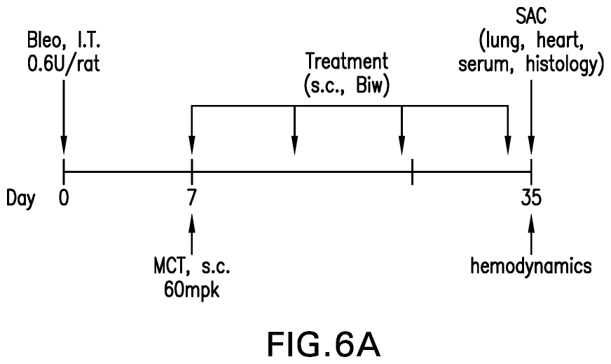

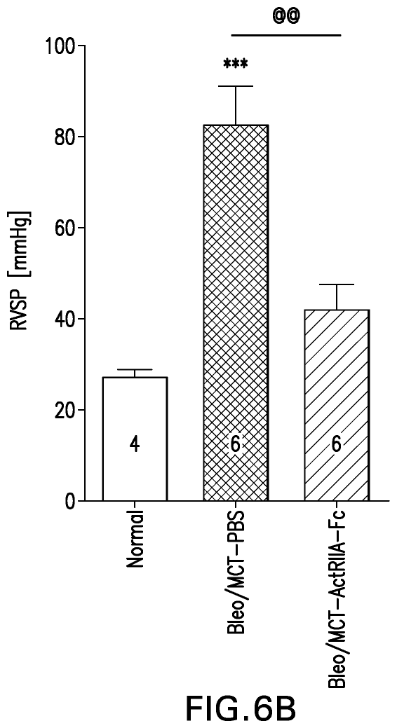

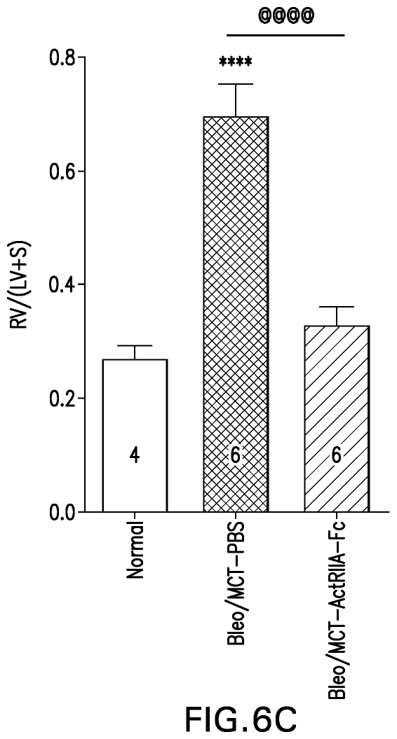

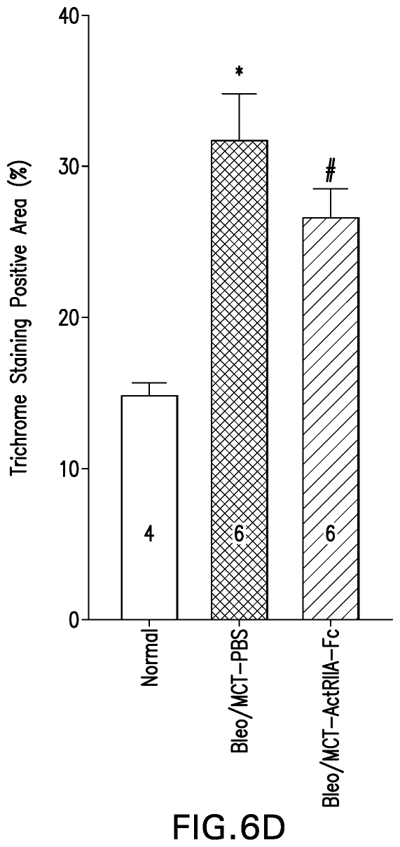

The effect of ActRIIA-mFc treatment of pulmonary hypertension and RV hypertrophy in Bleo-MCT PH-ILD rat model is shown in FIGS. 6A-6D. As shown in FIGS. 6B and 6C, Bleo-MCT treated rats (Bleo/MCT-PBS group) were observed to have elevated RVSP and right heart hypertrophy, indicating establishment of pulmonary hypertension and RV remodeling, compared to control animals. In addition, increased lung fibrosis was observed in Bleo-MCT rats (FIG. 6D). ActRIIA-mFc treatment significantly reduced increased RVSP (73%) and cardiac hypertrophy (87%). ActRIIA-mFc treatment also displayed a trend of decrease in lung fibrosis.

In another model, six Sprague-Dawley male rats were intratracheally administered with a single dose of bleomycin (Bleo, 0.6 U/rat) at day 0 and randomized into two treatment groups: 1) treatment with semaxanib (20 mg/kg administered s.c. as a single dose at day 7 of study)/hypoxia and Tris buffered saline (administered s.c. as 1 ml/kg, every three days) (Bleo/Su/Hx-PBS group), 2) treatment with semaxanib (20 mg/kg administered s.c. as a single dose at day 7 of study)/hypoxia and ActRIIA-mFc (5 mg/kg administered s.c. every three days). Rats were treated for 35 days. Body weights were recorded weekly throughout the study.

On day 42, rats were anesthetized with ˜3-4% isoflurane and placed on controlled heating pads. Right ventricular systolic pressure (RVSP) was measured by advancing a 2F curve tip pressure transducer catheter, (SPR-513, Millar Instruments) into the right ventricle (RV) via right jugular vein under ˜1.5-2% isofluorane anesthesia. RV hypertrophy was assessed by taking the weight ratio of RV free wall and LV+Septum (RV/LV+S, Fulton's Index).

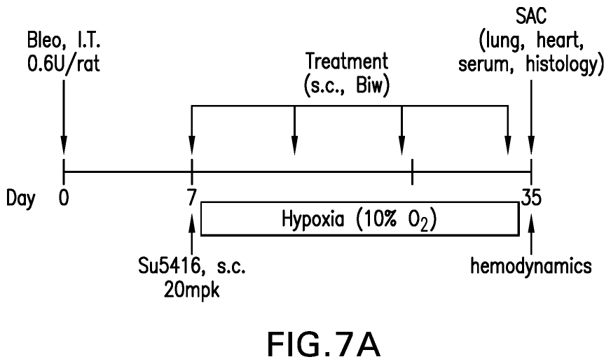

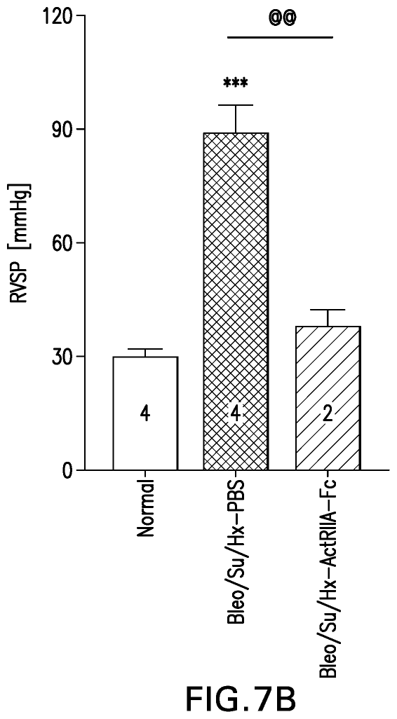

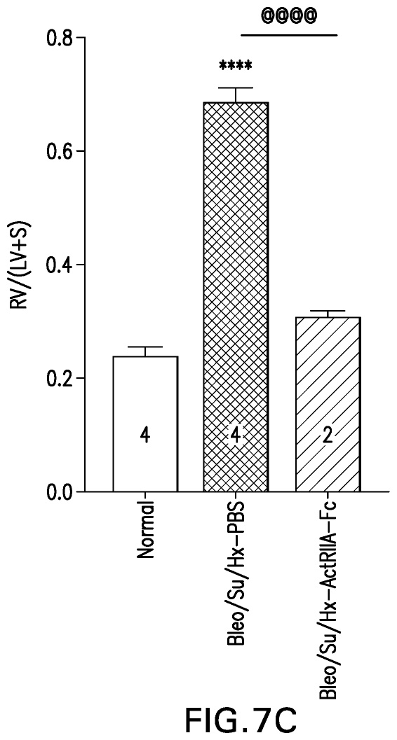

As shown in FIGS. 7A through 7C, Bleo-MCT treated rats (Bleo/Su/Hx-PBS group) were observed to have elevated RVSP and right heart hypertrophy, indicating establishment of pulmonary hypertension and RV remodeling, compared to control animals. ActRIIA-mFc treatment significantly reduced increased RVSP (87%) and cardiac hypertrophy (84%).

Together, these data demonstrate that ActRIIA-mFc is effective in ameliorating pulmonary hypertension in two bleomycin-induced Group 3 pulmonary hypertension models. In particular, ActRIIA-mFc had a significant effect in reducing RVSP and right heart hypertrophy. Furthermore, the data indicate that ActRIIA polypeptides, may be useful in the treatment of Group 3 PH, particularly in preventing or reducing the severity various complications of Group 3 PH.

### Example 5: Effects of an ActRIIA-mFc on Group 3 Pulmonary Hypertension in LPS Induced COPD Model of PH

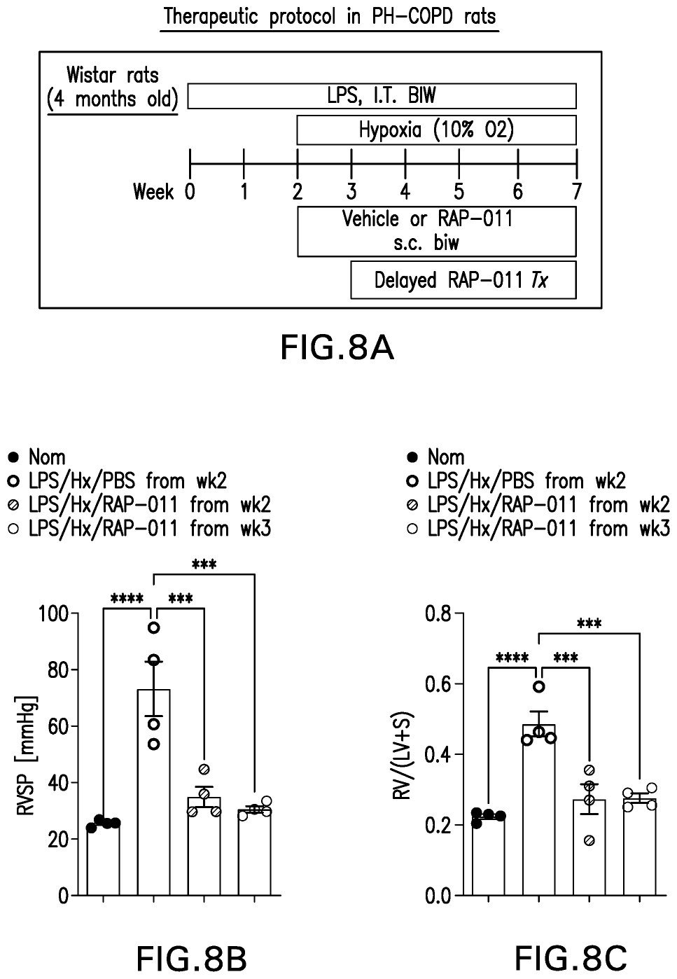

PH-COPD was induced in 12 Wistar male rats by bi-weekly intra-tracheal administration of lipopolysaccharides (LPS) (0.6 mg/mL diluted in 0.9% NaCl at 1 mL/kg bodyweight for 7 weeks and exposure to chronic hypoxia (10% 02) for 5 weeks after 2 weeks of intratracheal instillation of LPS. PH-COPD rats were randomized into three treatment groups: 1) Phosphate buffered saline (biw s.c. at 1 ml/kg from week 2-7) (vehicle treatment group), 2) ActRIIA-mFc (10 mg/kg administered s.c biw from week 2-7), and 3) ActRIIA-mFc (10 mg/kg administered s.c biw from week 3-7). Body weights were recorded weekly throughout the study. The therapeutic protocol is shown in FIG. 8A.

At week 7, rats were anesthetized with ˜3-4% isoflurane and placed on controlled heating pads. Right ventricular systolic pressure (RVSP) was measured by advancing a 2F curve tip pressure transducer catheter, (SPR-513, Millar Instruments) into the right ventricle (RV) via right jugular vein under ˜1.5-2% isofluorane anesthesia. RV hypertrophy was assessed by taking the weight ratio of RV free wall and LV+Septum (RV/LV+S, Fulton's Index).

As shown in FIGS. 8B and 8C, vehicle treated rats (treatment group 1) were observed to have elevated RVSP and right heart hypertrophy, indicating establishment of pulmonary hypertension and RV remodeling, compared to control animals. ActRIIA-mFc treatment significantly reduced increased RVSP by 80% in treatment group 2 and by 90% in treatment group 3. ActRIIA-mFc treatment significantly reduced increased RV hypertrophy by 84% in treatment group 2 and by 83% in treatment group 3.

These data demonstrate that ActRIIA-mFc is effective in ameliorating pulmonary hypertension in LPS/Hypoxia induced PH-COPD (Group 3 PH) rat pulmonary hypertension model. In particular, ActRIIA-mFc had a significant effect in reducing RVSP and right heart hypertrophy. Furthermore, the data indicate that ActRIIA polypeptides, may be useful in the treatment of Group 3 PH, particularly in preventing or reducing the severity various complications of Group 3 PH.

1-198. (canceled)

199. A method of treating pulmonary hypertension associated with lung disease, comprising administering to a patient in need thereof a fusion protein comprising:
a) an ActRIIA polypeptide comprising an amino acid sequence that is at least 95% identical to an amino acid sequence corresponding to residues 30-110 of SEQ ID NO: 1;
b) an Fc domain of an IgG1 immunoglobulin; and
c) a linker domain,
wherein the linker domain is positioned between the ActRIIA polypeptide and the Fc domain of the IgG1 immunoglobulin, and wherein the method reduces the right ventricular systolic pressure (RVSP) by at least 10%.

200. The method of claim 199, wherein the ActRII polypeptide comprises an amino acid sequence of SEQ ID NO: 2.

201. The method of claim 199, wherein the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21).

202. The method of claim 199, wherein the linker domain is TGGG (SEQ ID NO: 20).

203. The method of claim 199, wherein the Fc domain comprises an amino acid sequence that is at least 90% identical to the amino acid sequence of SEQ ID NO: 32.

204. The method of claim 199, wherein the Fc domain comprises an amino acid sequence that is at least 95% identical to the amino acid sequence of SEQ ID NO: 32.

205. The method of claim 199, wherein the Fc domain comprises an amino acid sequence that is at least 99% identical to the amino acid sequence of SEQ ID NO: 32.

206. The method of claim 199, wherein the fusion protein comprises an amino acid sequence that is at least 90% identical to the amino acid sequence of SEQ ID NO: 23.

207. The method of claim 199, wherein the fusion protein comprises an amino acid sequence that is at least 95% identical to the amino acid sequence of SEQ ID NO: 23.

208. The method of claim 199, wherein the fusion protein comprises an amino acid sequence that is at least 99% identical to the amino acid sequence of SEQ ID NO: 23.

209. The method of claim 199, wherein the fusion protein comprises an amino acid sequence of SEQ ID NO: 23.

210. The method of claim 199, wherein the fusion protein comprises an amino acid sequence that is at least 90% identical to the amino acid sequence of SEQ ID NO: 41.

211. The method of claim 199, wherein the fusion protein comprises an amino acid sequence that is at least 95% identical to the amino acid sequence of SEQ ID NO: 41.

212. The method of claim 199, wherein the fusion protein comprises an amino acid sequence that is at least 99% identical to the amino acid sequence of SEQ ID NO: 41.

213. The method of claim 199, wherein the fusion protein comprises an amino acid sequence of SEQ ID NO: 41.

214. The method of claim 199, wherein the fusion protein is part of a homodimer protein complex.

215. The method of claim 214, wherein the fusion protein is glycosylated.

216. The method of claim 199, wherein the fusion protein binds to one or more ligands selected from the group consisting of: activin A, activin B, GDF11, BMP10, GDF8, and BMP6.

217. A method of treating pulmonary hypertension associated with obstructive lung disease, comprising administering to a patient in need thereof a fusion protein comprising:
a) an ActRIIA polypeptide comprising an amino acid sequence that is at least 95% identical to an amino acid sequence corresponding to residues 30-110 of SEQ ID NO: 1;
b) an Fc domain of an IgG1 immunoglobulin; and
c) a linker domain,
wherein the linker domain is positioned between the ActRIIA polypeptide and the Fc domain of the IgG1 immunoglobulin.

218. The method of claim 217, wherein the ActRII polypeptide comprises an amino acid sequence of SEQ ID NO: 2.

219. The method of claim 217, wherein the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21).

220. The method of claim 217, wherein the linker domain is TGGG (SEQ ID NO: 20).

221. The method of claim 217, wherein the Fc domain comprises an amino acid sequence that is at least 90% identical to the amino acid sequence of SEQ ID NO: 32.

222. The method of claim 217, wherein the Fc domain comprises an amino acid sequence that is at least 95% identical to the amino acid sequence of SEQ ID NO: 32.

223. The method of claim 217, wherein the Fc domain comprises an amino acid sequence that is at least 99% identical to the amino acid sequence of SEQ ID NO: 32.

224. The method of claim 217, wherein the fusion protein comprises an amino acid sequence that is at least 90% identical to the amino acid sequence of SEQ ID NO: 23.

225. The method of claim 217, wherein the fusion protein comprises an amino acid sequence that is at least 95% identical to the amino acid sequence of SEQ ID NO: 23.

226. The method of claim 217, wherein the fusion protein comprises an amino acid sequence that is at least 99% identical to the amino acid sequence of SEQ ID NO: 23.

227. The method of claim 217, wherein the fusion protein comprises an amino acid sequence of SEQ ID NO: 23.

228. The method of claim 217, wherein the fusion protein comprises an amino acid sequence that is at least 90% identical to the amino acid sequence of SEQ ID NO: 41.

229. The method of claim 217, wherein the fusion protein comprises an amino acid sequence that is at least 95% identical to the amino acid sequence of SEQ ID NO: 41.

230. The method of claim 217, wherein the fusion protein comprises an amino acid sequence that is at least 99% identical to the amino acid sequence of SEQ ID NO: 41.

231. The method of claim 217, wherein the fusion protein comprises an amino acid sequence of SEQ ID NO: 41.

232. The method of claim 217, wherein the fusion protein is part of a homodimer protein complex.

233. The method of claim 232, wherein the fusion protein is glycosylated.

234. The method of claim 217, wherein the obstructive lung disease is selected from the group consisting of chronic obstructive pulmonary disease (COPD), cystic fibrosis, asthma, emphysema, lymphangioleiomyomatosis, and chronic bronchitis.

235. The method of claim 199, wherein the method prevents or reduces pulmonary hypertension Functional Class progression as recognized by the World Health Organization (WHO).


SEQUENCE LISTING (42 sequences)

SEQ ID NO: 1 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 2 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 3 - protein, 100 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 4 - DNA, 1539 bp, Homo sapiens
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 5 - DNA, 345 bp, Homo sapiens
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 6 - protein, 115 aa, Homo sapiens
  MOD_RES (45)..(45): Any amino acid
ILGRSETQECLFFNANWEKDRTNQTQVEPCYGDKDKRRHCFATWXNISGSIEIVKQGCWLDDINCYDRTDCVEKKDSPEVYFCCCEGNMCNEKFSTFPEMEVTQPTSNPVTPKPP

SEQ ID NO: 7 - protein, 115 aa, Ovis aries
  MOD_RES (45)..(45): Any amino acid
  MOD_RES (114)..(114): Any amino acid
ILGRSETQECLFYNANWERDRTNRTGVESCYGDKDKRRHCFATWXNISGSIDIVKQGCWLDDINCYDRTDCIEKKDSPEVYFCCCEGNMCNERFSTFPEMEVTQPTSNPVTPKXP

SEQ ID NO: 8 - protein, 115 aa, Unknown
  Description of Unknown: mole sp.
  MOD_RES (45)..(45): Any amino acid
  MOD_RES (113)..(113): Any amino acid
ILGRSETQECLFFNANWERDRTNQTGVEPCYGDKDKHRHCFATWXNISGSIEIVKQGCWLDDINCYDRTDCIEKKDSPEVYFCCCEGNMCNEKFSTFPEMEVTQPTSNPVTPXAP

SEQ ID NO: 9 - protein, 115 aa, Mus sp.
  MOD_RES (45)..(45): Any amino acid
  MOD_RES (68)..(68): Any amino acid
ILGRSETQECLFFNANWERDRTNQTGVEPCYGDKDKRRHCFATWXNISGSIEIVKQGCWLDDINCYDXTDCIEKKDSPEVYFCCCEGNMCNEKFSTFPEMEVTQPTSNPVTPKPP

SEQ ID NO: 10 - protein, 115 aa, Gallus gallus
  MOD_RES (45)..(45): Any amino acid
  MOD_RES (113)..(113): Any amino acid
ILGRSETQECLYYNANWEKDKTNRSGIEPCYGDKDKRRHCFATWXNISGSIEIVKQGCWLDDINCYDRNDCIEKKDSPEVFFCCCEGNMCNERFFTFPEMEVTQPTSNPVTPXPP

SEQ ID NO: 11 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 14 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 15 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 3 aa, Artificial Sequence
  Description of Artificial Sequence: Synthetic peptide
GGG

SEQ ID NO: 17 - protein, 4 aa, Artificial Sequence
  Description of Artificial Sequence: Synthetic peptide
GGGG

SEQ ID NO: 18 - protein, 5 aa, Artificial Sequence
  Description of Artificial Sequence: Synthetic peptide
TGGGG

SEQ ID NO: 19 - protein, 5 aa, Artificial Sequence
  Description of Artificial Sequence: Synthetic peptide
SGGGG

SEQ ID NO: 20 - protein, 4 aa, Artificial Sequence
  Description of Artificial Sequence: Synthetic peptide
TGGG

SEQ ID NO: 21 - protein, 4 aa, Artificial Sequence
  Description of Artificial Sequence: Synthetic peptide
SGGG

SEQ ID NO: 22 - protein, 5 aa, Artificial Sequence
  Description of Artificial Sequence: Synthetic peptide
GGGGS

SEQ ID NO: 23 - protein, 344 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 21 aa, Apis sp.
MKFLVNVALVFMVVYISYIYA

SEQ ID NO: 25 - protein, 22 aa, Homo sapiens
MDAMKRGLCCVLLLCGAVFVSP

SEQ ID NO: 26 - protein, 20 aa, Homo sapiens
MGAAAKLAFAVFLISCSSGA

SEQ ID NO: 27 - protein, 369 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 1114 bp, Artificial Sequence
  Description of Artificial Sequence: Synthetic polynucleotide
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 29 - protein, 9 aa, Artificial Sequence
  Description of Artificial Sequence: Synthetic peptide
ILGRSETQE

SEQ ID NO: 30 - protein, 329 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 32 - protein, 225 aa, Artificial Sequence; identical to SEQ ID NO: 11
  Description of Artificial Sequence: Synthetic polypeptide

SEQ ID NO: 33 - protein, 223 aa, Artificial Sequence
  Description of Artificial Sequence: Synthetic polypeptide
VECPPCPAPPVAGPSVFLFPPKPKDTLMISRTPEVTCVVVDVSHEDPEVQFNWYVDGVEVHNAKTKPREEQFNSTFRVVSVLTVLHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 232 aa, Artificial Sequence
  Description of Artificial Sequence: Synthetic polypeptide
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYKTTPPMLDSDGSFFLYSKLTVDKSRWQQGNIFSCSVMHEALHNRFTQKSLSLSPGK

SEQ ID NO: 35 - protein, 229 aa, Artificial Sequence; identical to SEQ ID NO: 15
  Description of Artificial Sequence: Synthetic polypeptide

SEQ ID NO: 36 - protein, 115 aa, Bos taurus
  MOD_RES (45)..(45): Any amino acid
  MOD_RES (113)..(113): Any amino acid
ILGRSETQECLFYNANWERDRTNRTGVESCYGDKDKRRHCFATWXNISGSIEIVKQGCWLDDINCYDRTDCIEKKDSPEVYFCCCEGNMCNERFSTFPEMEVTQPTSNPVTPXPP

SEQ ID NO: 37 - protein, 115 aa, Tyto alba; identical to SEQ ID NO: 10
  MOD_RES (45)..(45): Any amino acid
  MOD_RES (113)..(113): Any amino acid

SEQ ID NO: 38 - protein, 115 aa, Myotis davidii
  MOD_RES (45)..(45): Any amino acid
ILGRSETQECLFYNANWERDKTNRTGIEPCYGDKDKRRHCFATWXNISGSIEIVKQGCWLDDINCYDKTDCIEKKDSPEVYFCCCEGNMCNERFSTFPEMEVTQPTSNPVTPKPP

SEQ ID NO: 39-40 - skipped (2 entries)

SEQ ID NO: 41 - protein, 343 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 6 aa, Artificial Sequence
  Description of Artificial Sequence: Synthetic 6xhis tag
HHHHHH